(19) United States
(12) Patent Application Publication
(10) Pub. No.: US 2014/0057970 A1
(43) Pub. Date: Feb. 27, 2014

(54) Use of Hepcidin Binding Nucleic Acids for Depletion of Hepcidin From the Body

(71) Applicants: Frank Schwobel, Berlin (DE); John Turner, Berlin (DE); Nicola Klare, Berlin (DE); Sven Klussmann, Berlin (DE)

(72) Inventors: Frank Schwobel, Berlin (DE); John Turner, Berlin (DE); Nicola Klare, Berlin (DE); Sven Klussmann, Berlin (DE)

(21) Appl. No.: 13/882,197

(22) PCT Filed: Oct. 31, 2011
(86) PCT No.: PCT/EP2011/005498

§ 371 Date: Oct. 13, 2013

(30) Foreign Application Priority Data
Oct. 29, 2010 (EP) 10014124.1

(51) Int. Cl. C07H 21/02; C07H 1/06

(52) U.S. Cl. CPC C07H 21/02; C07H 1/06
USPC 514/44 R; 536/23.1; 210/646

(57) ABSTRACT

The present invention is related to a method for reducing the level of hepcidin in a body fluid from a subject, comprising
a) providing a nucleic acid molecule capable of binding to hepcidin, and
b) bringing the nucleic acid molecule into contact with a body fluid under conditions that allow for the binding of hepcidin to the nucleic acid molecule, thereby forming a complex of hepcidin and the nucleic acid molecule, and
c) removing the complex from the body fluid or removing the hepcidin from the body fluid.

The present invention relates to a method for reducing a hepcidin level in a body fluid, a nucleic acid molecule for use in such method, a medical device for use in such method, a nucleic acid molecule for use in a method for removing hepcidin from a body fluid of a subject, a nucleic acid molecule for use in a method for the treatment of an anaemic patient; and to a method for preparing a nucleic acid molecule immobilised to a support, whereby the thus prepared nucleic acid molecule may be used in any of said methods.

Bioactive hepcidin consists of 25 amino acids (also referred to as hepcidin-25). Additionally, two truncated inactive variants with 20 and 22 amino acids were identified: hepcidin-20 and hepcidin 22 (Rivera, 2005). The active 25 amino acids peptide hormone is found in blood and urine. Synonyms of the hepcidin are liver-expressed antimicrobial peptide (abbreviation: LEAP-1) and putative liver tumour regressor (abbreviation: PLTR) (Krause, 2000; Park 2001).

Hepcidin is the key signal regulating iron homeostatis. High levels of human hepcidin result in reduced serum iron levels whereas low levels result in increased serum iron levels as shown in hepcidin-deficiency and hepcidin overexpressing mouse models (Nicolas, 2001; Nicolas, 2002a; Nicolas, 2002b; Nicolas 2003). In addition, mutations in the hepcidin gene which result in lack of hepcidin activity are associated with juvenile hemochromatosis, a severe iron overload disease (Roetto, 2003). After intraperitoneal injection of hepcidin a dose dependent and long lasting reduction in serum iron was observed (Rivera, 2005).

Iron is an essential element required for growth and development of all living organisms. Iron content in mammals is regulated by controlling iron absorption, iron recycling, and release of iron from cells in which it is stored. Iron is absorbed predominantly in the duodenum and upper jejunum by enterocytes.

A feedback mechanism enhances iron adsorption in individuals who are iron deficient, and reduces iron absorption in individuals with iron overload. A key compound of this mechanism is the iron transporter ferroportin which also acts as hepcidin receptor and controls the release of iron (Abboud, 2000; Donovan, 2000; McKie, 2000). This major iron export protein is located on the basal membrane of placental syncytiotrophoblasts and enterocytes, and on the cell surface of macrophages and hepatocytes.

Hepcidin inhibits iron release from these different cell types by binding to ferroportin expressed on the above mentioned cell types and induces its phosphorylation, internalisation, ubiquitylation and lysosomal degradation thereby reducing ferroportin mediated release of iron into the blood (Nemeth, 2004b; De Domenico, 2007). As plasma iron continues to be consumed for haemoglobin synthesis, plasma iron levels decrease and hepcidin production abates in healthy subjects.

In situations of acute and chronic systemic inflammation cytokines induce hepcidin production. Hepcidin gene expression has been observed to be increased significantly after inflammatory stimuli, such as infections, which induce the acute phase response of the innate immune system of vertebrates. In mice hepcidin gene expression was shown to be upregulated by lipopolysaccharide (Constante, 2006), turpentine (Nemeth, 2004a) and Freund's complete adjuvant (Frazer, 2004), and adenoviral infections. In humans hepcidin expression is induced by the inflammatory cytokine interleukine-6 and LPS (Nemeth, 2004a). A strong correlation between hepcidin expression and anemia of inflammation was also found in patients with chronic inflammatory diseases, including bacterial, fungal and viral infections. In all these conditions increased concentrations of hepcidin inhibit iron efflux from macrophages, from hepatic storage and from duodenum into plasma. Hypoferremia develops, and erythopoiesis becomes iron-limited and results in anemia under conditions of chronic inflammation (Weiss, 2005; Weiss, 2008; Andrews, 2008).

A chronic inflammation can occur in the kidney and lead to chronic kidney disease, impaired kidney function and/or kidney failure. Anemia is common in people with kidney disease. Healthy kidneys produce a hormone called erythropoietin, or EPO, which stimulates the bone marrow to produce the proper number of red blood cells needed to carry oxygen to vital organs. Diseased kidneys, however, often don't make enough EPO. As a result, the bone marrow makes fewer red blood cells. Anemia develops even in the early stages of kidney disease, such as at 20 percent to 50 percent of normal kidney function. This partial loss of kidney function is often referred to as chronic renal insufficiency. Anemia worsens as kidney function deteriorates. End-stage kidney failure, the point at which dialysis or kidney transplantation becomes necessary, doesn't occur until there is only about 10 percent of your kidney function remaining. Nearly everyone with end-stage kidney failure has anemia.

Many patients with kidney disease need both EPO and iron supplements to raise their hematocrit to a satisfactory level. If a patient's iron levels are too low, EPO won't help and that person will continue to experience the effects of anemia. As a part of this therapy, reduction of hepcidin levels in the plasma or inactivation of hepcidin, preferably due to binding of hepcidin to a hepcidin binding or hepcidin inactivating compound so as to decrease its bioavailability, may also be beneficial for the patients. Reduction of hepcidin and inactivation of hepcidin will then result in macrophageal release of iron and improved absorption of iron.

However, if a hepcidin binding and inactivating compound, preferably a high molecular weight compound such as an antibody is administered to patients with impaired kidney function to reduce circulating levels of hepcidin, it is likely that the complex of hepcidin and the high molecular weight compound will not be rapidly excreted by the kidneys. In case an antibody is used for such purpose, due to secondary immune reactions triggered by the administered antibodies, such antibody therapy may lead to inflammation in the patients. As described before, such inflammation induces hepcidin production through cytokines.

Thus, the first problem underlying the present invention is to provide a compound which specifically interacts with hepcidin. More specifically, the problem underlying the present invention is to provide for a nucleic acid based compound which specifically interacts with hepcidin.

A further problem underlying the present invention is to provide means and methods for reducing the level of hepcidin in a body fluid of or from a subject, for removing hepcidin form a body fluid or a subject and/or for the treatment of an anaemic patient.

These and other problems underlying the present invention are solved by the subject matter of the attached independent claims. Preferred embodiments may be taken from the dependent claims.

More specifically, the problem underlying the present invention is solved in a first aspect which is also the first embodiment of the first aspect, by a method for reducing the level of hepcidin in a body fluid from a subject, comprising
a) providing a nucleic acid molecule capable of binding to hepcidin, and
b) bringing the nucleic acid molecule into contact with a body fluid under conditions that allow for the binding of hepcidin to the nucleic acid molecule, thereby forming a complex of hepcidin and the nucleic acid molecule, and
c) removing the complex from the body fluid or removing the hepcidin from the body fluid.

In a second embodiment of the first aspect which is also an embodiment of the first embodiment of the first aspect, the nucleic acid molecule comprises in 5′>3′ direction a first terminal stretch of nucleotides, a central stretch of nucleotides and a second terminal stretch of nucleotides, wherein the central stretch of nucleotides comprises 32 to 40 nucleotides, preferably 32 to 35 nucleotides.

In a third embodiment of the first aspect which is also an embodiment of the first embodiment of the first aspect, the nucleic acid molecule comprises 5′>3′ direction to a second terminal stretch of nucleotides, a central stretch of nucleotides and a first terminal stretch of nucleotides, wherein the central stretch of nucleotides comprises 32 to 40 nucleotides, preferably 32 to 35 nucleotides.

In a fourth embodiment of the first aspect which is also an embodiment of the second and the third embodiment of the first aspect, the central stretch of nucleotides is essential for the binding of the nucleic acid molecule to hepcidin.

In a fifth embodiment of the first aspect which is also an embodiment of the second, the third and the fourth embodiment of the first aspect, the central stretch of nucleotides comprises a nucleotide sequence of 5′ RKAUGGGAKUAAGUAAAUGAGGRGUWGGAGGAAR 3′ (SEQ.ID.No. 182) or 5′ RKAUGGGAKAAGUAAAUGAGGRGUWGGAGGAAR 3′ (SEQ.ID.No. 183).

In a sixth embodiment of the first aspect which is also an embodiment of the second, the third, the fourth and the fifth embodiment of the first aspect, the central stretch of nucleotides comprises a nucleotide sequence of 5′ RKAUGGGAKUAAGUAAAUGAGGRGUWGGAGGAAR 3′ (SEQ.ID.No. 182, preferably 5′ GUAUGGGAUUAAGUAAAUGAGGAGUUGGAGGAAG 3′(SEQ.ID.No. 184).

In a seventh embodiment of the first aspect which is also an embodiment of the fifth and the sixth embodiment of the first aspect,
the first terminal stretch of nucleotides comprises five to eight nucleotides, and
the second terminal stretch of nucleotides comprises five to eight nucleotides.

In an eighth embodiment of the first aspect which is also an embodiment of the fifth, the sixth and the seventh embodiment of the first aspect, the first terminal stretch of nucleotides and the second terminal stretch of nucleotides form a double-stranded structure, preferably such double-stranded structure is formed by the first terminal stretch of nucleotides and the second terminal stretch of nucleotides hybridizing to each other.

In a ninth embodiment of the first aspect which is also an embodiment of the fifth, the sixth, the seventh and the eighth embodiment of the first aspect, the first terminal stretch of nucleotides comprises a nucleotide sequence of 5′X1X2X3SBSBC3′ and the second terminal stretch of nucleotides comprises a nucleotide sequence of 5′GVBVYX4X5X6 3′,

wherein
X1 is A or absent, X2 is G or absent, X3 is B or absent, X4 is S or absent, X5 is C or absent, and X6 is U or absent.

In a tenth embodiment of the first aspect which is also an embodiment of the fifth, the sixth, the seventh, the eighth and the ninth embodiment of the first aspect, the first terminal stretch of nucleotides comprises a nucleotide sequence of 5′ X1X2X3SBSBC3′ and the second terminal stretch of nucleotides comprises a nucleotide sequence of 5′ GVBVBX4X5X6 3′,

wherein
a) X1 is A, X2 is G, X3 is B, X4 is S, X5 is C, and X6 is U; or
b) X1 is absent, X2 is G, X3 is B, X4 is S, X5 is C, and X6 is U; or
c) X1 is A, X2 is G, X3 is B, X4 is S, X5 is C, and X6 is absent.

In an eleventh embodiment of the first aspect which is also an embodiment of the fifth, the sixth, the seventh, the eighth, the ninth and the tenth embodiment of the first aspect,
a) the first terminal stretch of nucleotides comprises a nucleotide sequence of 5′ AGCGUGUC 3′ and the second terminal stretch of nucleotides comprises a nucleotide sequence of 5′ GGUGCGCU 3′; or
b) the first terminal stretch of nucleotides comprises a nucleotide sequence of 5′ AGCGUGUC 3′ and the second terminal stretch of nucleotides comprises a nucleotide sequence of 5′ GGCAUGCU 3′;
c) the first terminal stretch of nucleotides comprises a nucleotide sequence of 5′ AGUGUGUC 3′ and the second terminal stretch of nucleotides comprises a nucleotide sequence of 5′ GAUGCGCU 3′;
d) the first terminal stretch of nucleotides comprises a nucleotide sequence of 5′ AGUGUGUC 3′ and the second terminal stretch of nucleotides comprises a nucleotide sequence of 5′ GGCAUGCU 3′;
e) the first terminal stretch of nucleotides comprises a nucleotide sequence of 5′ AGCGUGCC 3′ and the second terminal stretch of nucleotides comprises a nucleotide sequence of 5′ GGUGCGCU 3′;
f) the first terminal stretch of nucleotides comprises a nucleotide sequence of 5′ AGCGCGCC 3′ and the second terminal stretch of nucleotides comprises a nucleotide sequence of 5′ GGCGCGCU 3′;

In an twelfth embodiment of the first aspect which is also an embodiment of the fifth, the sixth, the seventh, the eighth, and the ninth embodiment of the first aspect, the first terminal stretch of nucleotides comprises a nucleotide sequence of 5′X1X2X3SBSBC 3′ and the second terminal stretch of nucleotides comprises a nucleotide sequence of 5′ GVBVYX4X5X6 3′,

wherein
X1 is absent, X2 is G, X3 is B, X4 is S, X5 is C, and X6 is absent; or
X1 is absent, X2 is absent, X3 is B, X4 is S, X5 is C, and X6 is absent; or
X1 is absent, X2 is G, X3 is B, X4 is S, X5 is absent, and X6 is absent.

In a thirteenth embodiment of the first aspect which is also an embodiment of the fifth, the sixth, the seventh, the eighth, and the ninth embodiment of the first aspect, the first terminal stretch of nucleotides comprises a nucleotide sequence of 5′ X1X2X3SBSBC3′ and the second terminal stretch of nucleotides comprises a nucleotide sequence of 5′ GVBVYX4X5X6 3′,

wherein
X1 is absent, X2 is absent, X3 is absent, X4 is absent, X5 is absent, and X6 is absent.

In a fourteenth embodiment of the first aspect which is also an embodiment of the thirteenth embodiment of the first aspect,
a) the first terminal stretch of nucleotides comprises a nucleotide sequence of 5′ GCGCGC 3′ and the second terminal stretch of nucleotides comprises a nucleotide sequence of 5′ GCGCGC 3′; or
b) the first terminal stretch of nucleotides comprises a nucleotide sequence of 5′ GGUGUC 3′ and the second terminal stretch of nucleotides comprises a nucleotide sequence of 5′ GGCAUC 3′; or
c) the first terminal stretch of nucleotides comprises a nucleotide sequence of 5′ GGCGUC 3′ and the second terminal stretch of nucleotides comprises a nucleotide sequence of 5′ GGCGCC 3′;
d) the first terminal stretch of nucleotides comprises a nucleotide sequence of 5′ GCGCC 3′ and the second terminal stretch of nucleotides comprises a nucleotide sequence of 5′ GGCGC 3′; or
e) the first terminal stretch of nucleotides comprises a nucleotide sequence of 5′ GGCGC 3′ and the second terminal stretch of nucleotides comprises a nucleotide sequence of 5′ GCGCC 3′.

In a fifteenth embodiment of the first aspect which is also an embodiment of the first, the second, the third, the fourth, the fifth, the sixth, the seventh, the eighth, the ninth, the tenth, the eleventh, the twelfth, the thirteenth and the fourteenth embodiment of the first aspect, the nucleic acid comprises a nucleic acid sequence according to any one of SEQ.ID.Nos. 115 to 119, SEQ.ID.No. 121, SEQ.ID.No. 142, SEQ.ID.No. 144, SEQ.ID.No. 16, SEQ.ID.No. 148, SEQ.ID.No. 151, SEQ.ID.No. 152, SEQ.ID.No. 175, SEQ.ID.No. 176.

In a sixteenth embodiment of the first aspect which is also an embodiment of the second, the third and the fourth embodiment of the first aspect, the central stretch of nucleotides comprises a nucleotide sequence of 5′ GRCRGCCGGVGGACACCAUAUACAGACUACKAUA 3′ (SEQ.ID.No. 185) OR 5′ GRCRGCCGGVAGGACACCAUAUACAGACUACKAUA 3′ (SEQ.ID.No. 186).

In a seventeenth embodiment of the first aspect which is also an embodiment of the second, the third, the fourth and the sixteenth embodiment of the first aspect, the central stretch of nucleotides comprises a nucleotide sequence of 5′ GRCRGCCGGGGGACACCAUAUACAGACUACKAUA 3′ (SEQ.ID.No. 215), preferably 5′ GACAGCCGGGGGACACCAUAUACAGACUACGAUA 3′ (SEQ.ID.No. 187).

In an eighteenth embodiment of the first aspect which is also an embodiment of the sixteenth and the seventeenth embodiment of the first aspect,
the first terminal stretch of nucleotides comprises four to seven nucleotides, and
the second terminal stretch of nucleotides comprises four to seven nucleotides.

In a nineteenth embodiment of the first aspect which is also an embodiment of the eighteenth embodiment of the first aspect, the first terminal stretch of nucleotides and the second terminal stretch of nucleotides form a double-stranded structure, preferably such double-stranded structure is formed by the first terminal stretch of nucleotides and the second terminal stretch of nucleotides hybridizing to each other.

In a twentieth embodiment of the first aspect which is also an embodiment of the sixteenth, the seventeenth, the eighteenth and the nineteenth embodiment of the first aspect, the first terminal stretch of nucleotides comprises a nucleotide sequence of 5′ X1X2X3SBSN 3′ and the second terminal stretch of nucleotides comprises a nucleotide sequence of 5′ NSVSX4X5X6 3′,
wherein X1 is A or absent, X2 is G or absent, X3 is R or absent, X4 is Y or absent, X5 is C or absent, X6 is U or absent.

In a twenty first embodiment of the first aspect which is also an embodiment of the sixteenth, the seventeenth, the eighteenth, the nineteenth and the twentieth embodiment of the first aspect, the first terminal stretch of nucleotides comprises a nucleotide sequence of 5′ X1X2X3SBSN 3′ and the second terminal stretch of nucleotides comprises a nucleotide sequence of 5′ NSVSX4X5X6 3′,

wherein
a) X1 is A, X2 is G, X3 is R, X4 is Y, X5 is C, and X6 is U; or
b) X1 is absent, X2 is G, X3 is R, X4 is Y, X5 is C, and X6 is U; or
c) X1 is A, X2 is G, X3 is R, X4 is Y, X5 is C, and X6 is absent.

In a twenty second embodiment of the first aspect which is also an embodiment of the sixteenth, the seventeenth, the eighteenth, the nineteenth, the twentieth and the twenty first embodiment of the first aspect,
a) the first terminal stretch of nucleotides comprises a nucleotide sequence of 5′ AGGCUCG 3′ and the second terminal stretch of nucleotides comprises a nucleotide sequence of 5′ CGGGCCU 3′; or
b) the first terminal stretch of nucleotides comprises a nucleotide sequence of 5′ AGGCCCG 3′ and the second terminal stretch of nucleotides comprises a nucleotide sequence of 5′ CGGGCCU 3′; or
c) the first terminal stretch of nucleotides comprises a nucleotide sequence of 5′ AGGCUUG 3′ and the second terminal stretch of nucleotides comprises a nucleotide sequence of 5′ CGAGCCU 3′; or
d) the first terminal stretch of nucleotides comprises a nucleotide sequence of 5′ AGACUUG 3′ and the second terminal stretch of nucleotides comprises a nucleotide sequence of 5′ CGAGUCU 3′.

In a twenty third embodiment of the first aspect which is also an embodiment of the sixteenth, the seventeenth, the eighteenth, the nineteenth, and the twentieth embodiment of the first aspect, the first terminal stretch of nucleotides comprises a nucleotide sequence of 5′ X1X2X3SBSN 3′ and the second terminal stretch of nucleotides comprises a nucleotide sequence of ═ NSVSX4X5X6 3′,

wherein
a) X1 is absent, X2 is G, X3 is R, X4 is Y, X5 is C, and X6 is absent; or
b) X1 is absent, X2 is absent, X3 is R, X4 is Y, X5 is C, and X6 is absent; or
c) X1 is absent, X2 is G, X3 is R, X4 is Y, X5 is absent, and X6 is absent.

In a twenty fourth embodiment of the first aspect which is also an embodiment of the sixteenth, the seventeenth, the eighteenth, the nineteenth, the twentieth and the twenty third embodiment of the first aspect, the first terminal stretch of nucleotides comprises a nucleotide sequence of 5′ GGCUCG 3′ and the second terminal stretch of nucleotides comprises a nucleotide sequence of 5′ CGGGCC 3′.

In a twenty fifth embodiment of the first aspect which is also an embodiment of the sixteenth, the seventeenth, the eighteenth, the nineteenth, and the twentieth embodiment of the first aspect, the first terminal stretch of nucleotides comprises a nucleotide sequence of 5′ X1X2X3SBXN 3′ and the second terminal stretch of nucleotides comprises a nucleotide sequence of 5′ NSVSX4X5X6 3′,

wherein
X1 is absent, X2 is absent, X3 is R, X4 is Y or absent, X5 is absent, and X6 is absent.

In a twenty sixth embodiment of the first aspect which is also an embodiment of the sixteenth, the seventeenth, the eighteenth, the nineteenth, the twentieth and the twenty fifth embodiment of the first aspect,
the first terminal stretch of nucleotides comprises a nucleotide sequence of 5′ GGCCG 3′ and
the second terminal stretch of nucleotides comprises a nucleotide sequence of 5′ CGGCC 3′
   or
the first terminal stretch of nucleotides comprises a nucleotide sequence of 5′ GCGCG 3′
the second terminal stretch of nucleotides comprises a nucleotide sequence of 5′ CGCGC 3′.

In a twenty seventh embodiment of the first aspect which is also an embodiment of the first, the second, the third, the fourth, the sixteenth, the seventeenth, the eighteenth, the nineteenth, the twentieth and the twenty first, the twenty second, the twenty third, the twenty fourth, the twenty fifth and the twenty-sixth embodiment of the first aspect, the nucleic acid molecule comprises a nucleic acid sequence according to any one of SEQ.ID.Nos. 122 to 126, SEQ.ID.Nos. 154, SEQ.ID.Nos. 159, SEQ.ID.Nos. 163 SEQ.ID.Nos. 174.

In a twenty-eighth embodiment of the first aspect which is also an embodiment of the second, the third and the fourth embodiment of the first aspect, the central stretch of nucleotides comprises 5′>3′ direction the following stretches of nucleotides: a Box A, a linking stretch of nucleotides and a Box B; or a Box B, a linking stretch of nucleotides and a Box A, wherein the Box A comprises a nucleotide sequence of 5′ WAAAGUWGAR 3′ (SEQ.ID.Nos. 188), the linking stretch of nucleotides comprises ten to eighteen nucleotides and the Box B comprises a nucleotide sequence of 5′ RGMGUGWKAGUKC 3′ (SEQ.ID.Nos. 189).

In a twenty-ninth embodiment of the first aspect which is also an embodiment of the twenty-eighth embodiment of the first aspect, the Box A comprises a nucleotide sequence selected from the group of 5′ UAAAGUAGAG 3′ (SEQ.ID.Nos. 199), 5′ AAAAGUAGAA 3′ (SEQ.ID.Nos. 200), 5′ AAAAGUUGAA 3′ (SEQ.ID.Nos. 201) and 5′ GGGAUAUAGUGC 3′ (SEQ.ID.Nos. 202), preferably 5′ UAAAGUAGAG 3′ (SEQ.ID.Nos. 199).

In a thirtieth embodiment of the first aspect which is also an embodiment of the twenty-eighth and the twenty-ninth embodiment of the first aspect, the Box B comprises a nucleotide sequence selected from the group of 5′ GGCGUGAUAGUGC 3′ (SEQ.ID.Nos. 203), 5′ GGAGUGUUAGUUC 3′ (SEQ.ID.Nos. 204), 5′ GGCGUGAGAGUGC 3′ (SEQ.ID.Nos. 205), 5′ AGCGUGAUAGUGC 3′ (SEQ.ID.Nos. 206) and 5′ GGCGUGUUAGUGC 3′ (SEQ.ID.Nos. 207), preferably 5′ GGCGUGAUAGUGC 3′ (SEQ.ID.Nos. 203).

In a thirty-first embodiment of the first aspect which is also an embodiment of the twenty-eighth, the twenty-ninth and the thirtieth embodiment of the first aspect, the linking stretch of nucleotides comprises 5′>3′ direction a first linking substretch of nucleotides, a second linking substretch of nucleotides and a third linking substretch of nucleotides, wherein the first linking substretch of nucleotides and the third linking substretch of nucleotides each and independently from each other comprise three to six nucleotides.

In a thirty-second embodiment of the first aspect which is also an embodiment of the thirty-first embodiment of the first aspect, the first linking substretch of nucleotides and the third linking substretch of nucleotides form a double-stranded structure, preferably such double-stranded structure is formed by the first linking substretch of nucleotides and the third linking substretch of nucleotides hybridizing to each other.

In a thirty-third embodiment of the first aspect which is also an embodiment of the thirty-first, and the thirty-second embodiment of the first aspect, the double-stranded structure consists of three to six base pairs.

In a thirty-fourth embodiment of the first aspect which is also an embodiment of the thirty-first, the thirty-second and the thirty-third embodiment of the first aspect,
a) the first linking substretch of nucleotides comprises a nucleotide sequence of selected from the group of 5′ GGAC 3′, 5′GGAU + and 5′ GGA 3′, and the third linking substretch of nucleotides comprises a nucleotide sequence of 5′GUCC 3′; or
b) the first linking substretch of nucleotides comprises a nucleotide sequence of 5′ GGAG 3′ and the third linking substretch of nucleotides comprises a nucleotide sequence of 5′CUGC 3′; or
c) the first linking substretch of nucleotides comprises a nucleotide sequence of 5′ GGGC 3′ and the third linking substretch of nucleotides comprises a nucleotide sequence of 5′GCCC 3′; or
d) the first linking substretch of nucleotides comprises a nucleotide sequence of 5′ GAC 3′ and and the third linking substretch of nucleotides comprises a nucleotide sequence of 5′GUC 3′; or
e) the first linking substretch of nucleotides comprises a nucleotide sequence of 5′ ACUUGU 3′ and the third linking substretch of nucleotides comprises a nucleotide sequence selected from the group of 5′GCAAGU 3′ and 5′ GCAAGC 3′; or
f) the first linking substretch of nucleotides comprises a nucleotide sequence of 5′ UCCAG 3′ and the third linking substretch of nucleotides comprises a nucleotide sequence of 5′CUGGA 3′,
   preferably the first linking substretch of nucleotides comprises a nucleotide sequence of 5′ GAC 3′ and the third linking substretch of nucleotides comprises a nucleotide sequence of 5′ GUC 3′.

In a thirty-fifth embodiment of the first aspect which is also an embodiment of the thirty-first, the thirty-second, the thirty-third and the thirty-fourth embodiment of the first aspect, the second linking substretch of nucleotides comprises three to five nucleotides.

In a thirty-sixth embodiment of the first aspect which is also an embodiment of the thirty-first, thirty-second, the thirty-third, the thirty-fourth and the thirty-fifth embodiment of the first aspect, the second linking substretch of nucleotides comprises a nucleotide sequence selected from the group of 5′ VBAAW 3′, 5′ AAUW 3′ and 5′ NBW 3′.

In a thirty-seventh embodiment of the first aspect which is also an embodiment of the thirty-sixth embodiment of the first aspect, the second linking substretch of nucleotides comprises a nucleotide sequence of 5′ VBAAW 3′, preferably a nucleotide sequence selected from the group of 5′ CGAAA 3′, 5′ GCAAU 3′, 5′ GUAAU 3′ and 5′ AUAAU 3′.

In a thirty-eighth embodiment of the first aspect which is also an embodiment of the thirty-sixth embodiment of the first aspect, the second linking substretch of nucleotides comprises a nucleotide sequence of 5′ AAUW 3′, preferably a nucleotide sequence of 5′ AAUU3′ or 5′ AAUA 3′, more preferably 5′ AAUA 3′.

In a thirty-ninth embodiment of the first aspect which is also an embodiment of the thirty-sixth embodiment of the first aspect, the second linking substretch of nucleotides comprises a nucleotide sequence of 5′ NBW 3′, preferably the second linking substretch of nucleotides comprises a nucleotide sequence selected from the group of 5′ CCA 3′, 5′CUA 3′, 5′ UCA 3′, 5′ ACA 3′, 5′ GUU 3′, 5′ UGA 3′ and 5′ GUA 3′, more preferably 5′ CCA 3′, 5′ CUA 3′, 5′ UCA 3′, 5′ ACA 3′ and 5′ GUU 3′.

In a fortieth embodiment of the first aspect which is also an embodiment of the thirty-eighth, the twenty-ninth, the thirtieth, the thirty-first, the thirty-second, the thirty-third, the thirty-fourth, the thirty-fifth, the thirty-sixth, the thirty-seventh, the thirty-eighth and the thirty-ninth embodiment of the first aspect, the second linking stretch of nucleotides comprises a nucleotide sequence selected from the group of 5′ GGACBYAGUCC 3′ (SEQ.ID.NO. 208), 5′ GGAUACAGUCC 3′ (SEQ.ID.NO. 209), 5′GCAGGYAAUCUGC 3′ (SEQ.ID.NO. 210), 5′ GACAAUWGUC 3′ (SEQ.ID.NO. 211), 5′ ACUUGUCGAAAGCAAGY 3′ (SEQ.ID.NO. 212), 5′ UCCAGGUUCUGGA 3′ (SEQ.ID.NO. 109), 5′ GGGCUGAGCCC 3′ (SEQ.ID.NO. 190), 5′ GCAGAUAAUCUGC 3′ (SEQ.ID.NO. 191), and 5′ GGACCAGUCC 3′ (SEQ.ID.NO. 192), preferably wherein the linking stretch of nucleotides comprises a nucleotide sequence selected from the group of 5′ GGACCCAGUCC 3′ (SEQ.ID.NO. 193), 5′ GGACCUAGUCC 3′ (SEQ.ID.NO. 194), 5′ GGACUCAGUCC 3′ (SEQ.ID.NO. 195), 5′ GGACGUAGUCC 3′ (SEQ.ID.NO. 214), 5′ GCAGGUAAUCUGC 3′ (SEQ.ID.NO. 196), 5′ GCAGGCAAUCUGC 3′ (SEQ.ID.NO. 197), 5′ GACAAUUGUC 3′ (SEQ.ID.NO. 198), and 5′ GACAAUAGUC 3′ (SEQ.ID.NO. 157).

In a forty-first embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, and the fortieth embodiment of the first aspect,
the first terminals stretch of nucleotides comprises four to seven nucleotides, and
the second terminal stretch of nucleotides comprises four to seven nucleotides.

In a forty-second embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth and the forty-first embodiment of the first aspect, the first terminal stretch of nucleotides and the second terminal stretch of nucleotides form a double-stranded structure, preferably such double-stranded structure is formed by the first terminal stretch of nucleotides and the second terminal stretch of nucleotides hybridizing to each other.

In a forty-third embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first and the forty-second embodiment of the first aspect, the first terminal stretch of nucleotides comprises a nucleotide sequence of 5′ X1X2X3BKBK 3′ and the second terminal stretch of nucleotides comprises a nucleotide sequence of 5′ MVVVX4X5X6 3′,

wherein X1 is G or absent, X2 is S or absent, X3 is V or absent, X4 is B or absent, X5 is S or absent, X6 is C or absent.

In a forty-fourth embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second and the forty-third embodiment of the first aspect, the first terminal stretch of nucleotides comprises a nucleotide sequence of 5′ X1X2X3BKBK 3′ and the second terminal stretch of nucleotides comprises a nucleotide sequence of 5′ MVVVX4X5X6 3′,

wherein
a) X1 is G, X2 is S, X3 is V, X4 is B, X5 is S, and X6 is C or
b) X1 is absent, X2 is S, X3 is V, X4 is B, X5 is S, and X6 is C or
c) X1 is G, X2 is S, X3 is V, X4 is B, X5 is S, and X6 is absent.

In a forty-fifth embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty third and the forty-fourth embodiment of the first aspect, preferably of the forty-fourth embodiment of the first aspect, the first terminal stretch of nucleotides comprises a nucleotide sequence of 5′ GCACUCG 3′ and the second terminal stretch of nucleotides comprises a nucleotide sequence of 5′ CGAGUGC 3′.

In a forty-fifth embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, and the forty-third embodiment of the first aspect, the first terminal stretch of nucleotides comprises a nucleotide sequence of 5′ X1X2X3BKBK 3′ and the second terminal stretch of nucleotides comprises a nucleotide sequence of 5′ MVVVX4X5X6 3′,

wherein
a) X1 is absent, X2 is S, X3 is V, X4 is B, X5 is S, and X6 is absent or
b) X1 is absent, X2 is absent, X3 is V, X4 is B, X5 is S, and X6 is absent or
c) X1 is absent, X2 is S, X3 is V, X4 is B, X5 is absent, and X6 is absent.

In a forty-seventh embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty third and the forty-sixth embodiment of the first aspect,

a) the first terminal stretch of nucleotides comprises a nucleotide sequence of 5′ GCUGUG 3′ and the second terminal stretch of nucleotides comprises a nucleotide sequence of 5′ CACAGC 3′; or

b) the first terminal stretch of nucleotides comprises a nucleotide sequence of 5′ CGUGUG 3′ and the second terminal stretch of nucleotides comprises a nucleotide sequence of 5′ CACACG 3′; or

c) the first terminal stretch of nucleotides comprises a nucleotide sequence of 5′ CGUGCU 3′ and the second terminal stretch of nucleotides comprises a nucleotide sequence of 5′ AGCACG 3′; or

d) the first terminal stretch of nucleotides comprises a nucleotide sequence of 5′ CGCGCG 3′ and the second terminal stretch of nucleotides comprises a nucleotide sequence of 5′ CGCGCG 3′; or

e) the first terminal stretch of nucleotides comprises a nucleotide sequence of 5′ GCCGUG 3′ and the second terminal stretch of nucleotides comprises a nucleotide sequence of 5′ CACGCG 3′; or

f) the first terminal stretch of nucleotides comprises a nucleotide sequence of 5′ GCGGUG 3′ and the second terminal stretch of nucleotides comprises a nucleotide sequence of 5′ CACCGC 3′; or

g) the first terminal stretch of nucleotides comprises a nucleotide sequence of 5′ GCUGCGG 3′ and the second terminal stretch of nucleotides comprises a nucleotide sequence of 5′ CGCAGC 3′; or

h) the first terminal stretch of nucleotides comprises a nucleotide sequence of 5′ GCUGGG 3′ and the second terminal stretch of nucleotides comprises a nucleotide sequence of 5′ CCCAGC 3′; or

i) the first terminal stretch of nucleotides comprises a nucleotide sequence of 5′ GCGGCG 3′ and the second terminal stretch of nucleotides comprises a nucleotide sequence of 5′ CGCCGC 3′.

In a forty-eighth embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, and the forty-third embodiment of the first aspect, the first terminal stretch of nucleotides comprises a nucleotide sequence of 5′ X1X2X3BKBK 3′, and the second terminal stretch of nucleotides comprises a nucleotide sequence of 5′ MVVVX4X5X6 3′,
wherein X1 is absent, X2 is absent, X3 is V or absent, X4 is B or absent, X5 is absent and X6 is absent.

In a forty-ninth embodiment of the first aspect which is also an embodiment of the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third and the forty-eighth embodiment of the first aspect, the first terminal stretch of nucleotides comprises a nucleotide sequence of 5′ CGUG 3′ and the second terminal stretch of nucleotides comprises a nucleotide sequence of 5′ CACG 3′.

In a fiftieth embodiment of the first aspect which is also an embodiment of the firs, the second, the third, the fourth, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth and the forty-ninth embodiment of the first aspect, the nucleic acid molecule comprises a nucleic acid sequence according to any one of SEQ.ID No. 29, SEQ.ID No. 33, SEQ.ID No. 34, SEQ.ID Nos. 39 or 41, SEQ.ID No. 43, SEQ.ID No. 46, SEQ.ID Nos. 137 to 141 or SEQ.ID No. 173.

In a fifty-first embodiment of the first aspect which is also an embodiment of the first embodiment of the first aspect, the nucleic acid molecule comprises a nucleic acid sequence according to any one of SEQ.ID.Nos. 127 to 131.

In a fifty-second embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty-first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty seventh, the forty-eighth, the forty-ninth, the fiftieth and the fifty-first embodiment of the first aspect, the hepcidin is human hepcidin-25, human hepcidin-22 human hepcidin-20, monkey hepcidin-25, monkey hepcidin-22, or monkey hepcidin-20, preferably human hepcidin-25.

In a fifty-third embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty-first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty seventh, the forty-eighth, the forty-ninth, the fiftieth, the fifty-first and and the fifty-second embodiment of the first aspect, preferably of the fifty-second embodiment of the first aspect, the hepcidin has an amino acid sequence according to SEQ ID No. 1.

In a fifty-fourth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty seventh, the forty-eighth, the forty-ninth, the fiftieth, the fifty-first, the fifty-second and the fifty-third embodiment of the first aspect, the nucleic acid molecule comprises a modification group, wherein preferably excretion rate of the nucleic acid molecule comprising the modification group from an organism is decreased compared to a nucleic acid molecule capable of binding to hepcidin not comprising the modification group.

In a fifty-fifth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty seventh, the forty-eighth, the forty-ninth, the fiftieth, the fifty-first, the fifty-second and the fifty-third embodiment of the first aspect, the nucleic acid molecule comprises a modification group, wherein preferably the nucleic acid molecule comprising the modification group has an increased retention time in an organism compared to a nucleic acid molecule capable of binding to hepcidin not comprising the modification group.

In a fifty-sixth embodiment of the first aspect which is also an embodiment of the fifty-fourth and the fifty-fifth embodiment of the first aspect, the modification group is selected from the group comprising biodegradable and non-biodegradable modifications, preferably the modification group is selected from the group comprising linear poly(ethylene)glycol, branched poly(ethylene)glycol, hydroxethyl starch, a peptide, a protein, a polysaccharide, a sterol, polyoxypropylene, polyoxyamidate, poly(2-hydroxethyl)-L-glutamine and polyethylene glycol.

In a fifty-seventh embodiment of the first aspect which is also an embodiment of the fifty-sixth embodiment of the first aspect, the modification group is a PEG moiety consisting of a straight poly(ethylene)glycol or branched poly(ethylene)glycol, wherein the molecule weight of the poly(ethylene)glycol moiety is preferably from about 20,000 to about 120,000 Da, more preferably from about 30,000 to about 80,000 Da and most preferably about 40,000 Da.

In a fifty-eighth embodiment of the first aspect which is also an embodiment of the fifty-sixth embodiment of the first aspect, the modification group is a hydroxyethyl starch moiety, wherein preferably the molecular weight of the hydroxyethyl starch moiety is from about 10,000 to about 200,000 Da, more preferably from about 30,000 to about 170,000 Da and most preferably about 150,000 Da.

In a fifty-ninth embodiment of the first aspect which is also an embodiment of the fifty-fourth, the fifty-fifth, the fifty-sixth, the fifty-seventh and the fifty-eighth embodiment of the first aspect, the modification group is coupled to the nucleic acid molecule via a linker, whereby preferably the linker is a biodegradable linker.

In a sixtieth embodiment of the first aspect which is also an embodiment of the fifty-fourth, the fifty-fifth, the fifty-sixth, the fifty-seventh, the fifty-eighth and the fifty-ninth embodiment of the first aspect, the modification group is coupled to the 5′-terminal nucleotide and/or the 3′-terminal nucleotide of the nucleic acid molecule and/or to a nucleotide of the nucleic acid molecule between the 5′-terminal nucleotide of the nucleic acid molecule and the 3′-terminal nucleotide of the nucleic acid molecule.

In a sixtieth-first embodiment of the first aspect which is also an embodiment of the fifty-fourth, the fifty-fifth, the fifty-sixth, the fifty-seventh, the fifty-eighth, the fifty-ninth and the sixtieth embodiment of the first aspect, the organism is an animal body or a human body, preferably a human body.

In a sixty-second embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth and the sixty-first embodiment of the first aspect, the nucleotides of or the nucleotides forming the nucleic acid molecule are L-nucleotides.

In a sixty-third embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first and the sixty-second embodiment of the first aspect, the nucleic acid molecule is an L-nucleic acid.

In a sixty-fourth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second and the sixty-third embodiment of the first aspect, the nucleic acid molecule comprises at least one binding moiety which is capable of binding hepcidin, wherein such binding moiety consists of L-nucleotides.

In a sixty-fifth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, and the sixty-fourth embodiment of the first aspect, the method is a method for the treatment and/or prevention of a disease or wherein the method is part of a method for the treatment and/or prevention of a disease, or used for or in connection with such method.

In a sixty-sixth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth and the sixty-fifth embodiment of the first aspect, the nucleic acid molecule is immobilized on a support.

In a sixty-seventh embodiment of the first aspect which is also an embodiment of the sixty-sixth embodiment of the first aspect, the nucleic acid molecule immobilized on a support is located ex vivo.

In a sixty-eighth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth and the sixty-fifth and embodiment of the first aspect, the nucleic acid molecule comprises a modification group as defined in any one of the fifty-fourth, the fifty-fifth, the fifty-sixth, the fifty-seventh, the fifty-eighth, the fifty-ninth and the sixtieth embodiment of the first aspect and is thereby modified thus forming a modified nucleic acid molecule.

In a sixtieth-ninth embodiment of the first aspect which is also an embodiment of the sixty-eighth embodiment of the first aspect, the complex of hepcidin and the modified nucleic acid molecule is brought into contact with a ligand for the modification group, and thereby removing the complex from the body fluid.

In a seventieth embodiment of the first aspect which is also an embodiment of the sixty-eighth and the sixth-ninth embodiment of the first aspect, the modified nucleic acid molecule is part of a pharmaceutical composition comprising the modified nucleic acid molecule and optionally a further consistent, wherein the further constituent is selected from the group comprising pharmaceutically acceptable excipients, pharmaceutically acceptable carriers and pharmaceutically active agents.

In a seventy-first embodiment of the first aspect which is also an embodiment of the sixty-ninth and the seventieth embodiment of the first aspect, the ligand is immobilized on a support and is located ex vivo.

In a seventy-second embodiment of the first aspect which is also an embodiment of the sixty-sixth and the sixty-seventh embodiment of the first aspect, the nucleic acid molecule is immobilised on the support by the 3′ terminus of or the 5′ of said nucleic acid.

In a seventy-third embodiment of the first aspect which is also an embodiment of the sixty-sixth, the sixty-seventh, the sixty-eighth, the sixty-ninth, the seventieth, the seventy-first and the seventy-second embodiment of the first aspect, the nucleic acid molecule or the ligand is immobilised by covalent binding, non-covalent binding, hydrogen bonding, van der Waals interactions, coulombic interaction, hydrophobic interaction or coordinate binding.

In a seventy-fourth embodiment of the first aspect which is also an embodiment of the sixty-sixth, the sixty-seventh, the seventy-first, the seventy-second and the seventy-third embodiment of the first aspect, the support is a solid support, preferably comprising an organic polymer and/or an inorganic polymer.

In a seventy-fifth embodiment of the first aspect which is also an embodiment of the seventy-fourth embodiment of the first aspect, the solid support is selected from the group consisting of controlled pore glass, clay, cellulose, dextran, acrylics, agarose, polystyrene, sepharose, silica beads, and acrylate base amino support and a methacrylate base amino support.

In a seventy-sixth embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth and the sixty-fifth embodiment of the first aspect, the body fluid is brought into contact with a semi-permeable membrane that separates the body fluid from a dialysate such that hepcidin and/or the complex of hepcidin and the nucleic acid molecule diffuses through the semi-permeable membrane from the body fluid to the dialysate, thereby reducing the level of hepcidin in the body fluid.

In a seventy-seventh embodiment of the first aspect which is also an embodiment of the seventy-sixth embodiment of the first aspect, the nucleic acid molecule is not modified.

In a seventy-eighth embodiment of the first aspect which is also an embodiment of the seventy-sixth, and the seventy-seventh embodiment of the first aspect, the complex of hepcidin and the not modified nucleic acid molecule diffuses from the body fluid to the dialysate.

In a seventy-ninth embodiment of the first aspect which is also an embodiment of the seventy-sixth embodiment of the first aspect, the nucleic acid molecule comprises a modification group as defined in any one of the fifty-fourth, the fifty-fifth, the fifty-sixth, the fifty-seventh, the fifty-eighth, the fifty-ninth and the sixtieth embodiment of the first aspect thus forming a modified nucleic acid molecule.

In an eightieth embodiment of the first aspect which is also an embodiment of the seventy-ninth embodiment of the first aspect, the modified nucleic acid molecule is present in the dialysate.

In an eighty-first embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth and the eightieth embodiment of the first aspect, the body fluid is blood, plasma or serum.

In an eighty-second embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth and the eighty-first embodiment of the first aspect, said subject has impaired kidney function.

In an eighty-third embodiment of the first aspect which is also an embodiment of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first and the eighty-second embodiment of the first aspect, the method is an ex vivo method.

In an eighty-fourth embodiment of the first aspect which is also an embodiment of the eighty-third embodiment of the first aspect, the body fluid is not returned to the body from which the body fluid is or has been taken.

In a eighty-fifth embodiment of the first aspect which is also an embodiment of the eighty-third and the eighty-fourth embodiment of the first aspect, the body fluid is a blood reserve.

The problem underlying the present invention is solved in a second aspect which is also the first embodiment of the second aspect, by a method for preparing a nucleic acid molecule immobilised to a support wherein the nucleic acid molecule is capable of binding to hepcidin, wherein the method comprises: reacting a nucleic acid molecule capable of binding to hepcidin and an activated support to form a bond between a 3′ end, a 5′ end or both of the nucleic acid molecule and the support.

In a second embodiment of the second aspect which is also an embodiment of the first embodiment of the second aspect, the method further comprises blocking the activated support to prevent any covalent binding of components of body fluids other then hepcidin.

In a third embodiment of the second aspect which is also an embodiment of the first and the second embodiment of the second aspect, the support is a sepharose.

In a fourth embodiment of the second aspect which is also an embodiment of the third embodiment of the second aspect, the sepharose support is activated using a NHS-ester.

In a fifth embodiment of the second aspect which is also an embodiment of the fourth embodiment of the second aspect, the activated sepharose support is blocked by treatment with ethanolamine.

In a sixth embodiment of the second aspect which is also an embodiment of the first, the second, the third, the fourth and the fifth embodiment of the second aspect, the nucleic acid molecule comprises an amino-functional moiety and the nucleic acid molecule is immobilized to an activated sepharose support in a mildly basic solution

In a seventh embodiment of the second aspect which is also an embodiment of the sixth embodiment of the second aspect, the amino functional moiety comprises three hexaethylene glycol moieties, wherein the three hexaethylene glycol moieties are arranged between the nucleic acid molecule and the amino functional moiety.

The problem underlying the present invention is solved in a third aspect which is also the first embodiment of the third aspect, by a nucleic acid molecule immobilised on a support, wherein the nucleic acid molecule is capable of binding to hepcidin.

In a second embodiment of the third aspect which is also an embodiment of the first embodiment of the third aspect, the nucleic acid molecule is or comprises a nucleic acid molecule as described in any one of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth and the seventy-fifth embodiment of the first aspect.

The problem underlying the present invention is solved in a fourth aspect which is also the first embodiment of the fourth aspect, by a medical device for use in a method according to any of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first, the eighty-second, the eighty-third, the eighty-fourth and the eighty-fifth embodiment of the first aspect.

In a second embodiment of the fourth aspect which is also an embodiment of the first embodiment of the fourth aspect, the device comprises a nucleic acid molecule as defined in any one of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first, the eighty-first, the eighty-second, the eighty-third, the eighty-fourth and the eighty-fifth embodiment of the first aspect and/or a nucleic acid molecule according to any one of the first and the second embodiment of the third aspect.

The problem underlying the present invention is solved in a fifth aspect which is also the first embodiment of the fifth aspect, by a nucleic acid molecule for use in a method for reducing the level of hepcidin in a body fluid of a subject, preferably a mammal and more preferably a human being, wherein the nucleic acid is a nucleic acid molecule capable of binding to hepcidin.

In a second embodiment of the fifth aspect which is also an embodiment of the first embodiment of the fifth aspect, the nucleic acid molecule is or comprises a nucleic acid molecule as described in any one of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first, the eighty-first, the eighty-second, the eighty-third, the eighty-fourth and the eighty-fifth embodiment of the first aspect.

The problem underlying the present invention is solved in a sixth aspect which is also the first embodiment of the sixth aspect, by a nucleic acid molecule for use in a method for removing hepcidin form a body fluid of a subject, wherein the nucleic acid molecule is a nucleic acid molecule as defined in any one of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first, the eighty-second, the eighty-third, the eighty-fourth and the eighty-fifth embodiment of the first aspect.

In a second embodiment of the sixth aspect which is also an embodiment of the first embodiment of the sixth aspect, the method as defined in any one of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first, the eighty-second, the eighty-third, the eighty-fourth and the eighty-fifth embodiment of the first aspect.

The problem underlying the present invention is solved in a seventh aspect which is also the first embodiment of the seventh aspect, by a nucleic acid molecule for use in a method for the treatment of an anaemic patient, wherein the nucleic acid molecule is a nucleic acid molecule as defined in any one of the first, the second, the third, the fourth, the fifth, the sixth, the seventh, the eighth, the ninth, the tenth, the eleventh, the twelfth, the thirteenth, the fourteenth, the fifteenth, the sixteenth, the seventeenth, the eighteenth, the nineteenth, the twentieth, the twenty first, the twenty second, the twenty third, the twenty fourth, the twenty fifth, the twenty-sixth, the twenty seventh, the twenty-eighth, the twenty-ninth, the thirtieth, the thirty-first, the thirty-second, the thirty-third, the thirty-fourth, the thirty-fifth, the thirty-sixth, the thirty-seventh, the thirty-eighth, the thirty-ninth, the fortieth, the forty-first, the forty-second, the forty-third, the forty-fourth, the forty-fifth, the forty-sixth, the forty-seventh, the forty-eighth, the forty-ninth, the fiftieth, the fifty-first, the fifty-second, the fifty-third, the fifty-fourth, the fifty-fifth, the fifty-sixth, the fifty-seventh, the fifty-eighth, the fifty-ninth, the sixtieth, the sixty-first, the sixty-second, the sixty-third, the sixty-fourth, the sixty-fifth, the sixty-sixth, the sixty-seventh, the sixty-eighth, the sixty-ninth, the seventieth, the seventy-first, the seventy-second, the seventy-third, the seventy-fourth, the seventy-fifth, the seventy-sixth, the seventy-seventh, the seventy-eighth, the seventy-ninth, the eightieth, the eighty-first, the eighty-first, eighty-second, the eighty-third, the eighty-fourth and the eighty-fifth embodiment of the first aspect.

In a second embodiment of the seventh aspect which is also an embodiment of the first embodiment of the seventh aspect, the treatment comprises elimination of hepcidin from a body fluid of the patient, preferably by interaction of hepcidin with the nucleic acid molecule.

In a third embodiment of the seventh aspect which is also an embodiment of the second embodiment of the seventh aspect, the nucleic acid molecule is present, preferably immobilised, in an extracorporal device and the body fluid is removed from the body of the patient, passed through the extracorporal device and returned into the body of the patient.

In a fourth embodiment of the seventh aspect which is also an embodiment of the third embodiment of the seventh aspect, the body fluid is blood or blood plasma.

The features of the nucleic acid according to the present invention as described herein can be realised in any aspect of the present invention where the nucleic acid is used, either alone or in any combination.

The present invention is based on the surprising finding that it is possible to generate nucleic acids as a compound binding specifically and with high affinity to hepcidin, whereby human hepcidin-25 is a basic protein having the amino acid sequence according to SEQ. ID Nos. 1.

It is within the present invention that the nucleic acid according to the present invention is a nucleic acid molecule. Insofar the terms nucleic acid and nucleic acid molecule are used herein in a synonymous manner if not indicated to the contrary. Moreover, such nucleic acids are preferably also referred to herein as the nucleic acid molecules according to the present invention, the nucleic acids according to the present invention, the inventive nucleic acids or the inventive nucleic acid molecules.

As outlined in more detail in the claims and example 1, the present inventors could more surprisingly identify a number of different nucleic acid molecules capable of binding to hepcidin, whereby most of the nucleic acids could be characterised in terms of stretches of nucleotides which are also referred to herein as Boxes. The various nucleic acid molecules capable of binding to hepcidin can be categorised as Type A, Type B and Type C hepcidin binding nucleic acids based on said Boxes and some additional structural features and elements, respectively.

The different types of hepcidin binding nucleic acids comprise different stretches of nucleotides. Accordingly, the different types of hepcidin binding nucleic acids show a different binding behaviour to the different hepcidin peptides. As demonstrated in the Examples hepcidin binding nucleic acids according to the present invention bind to human hepcidin-25, human hepcidin-22, human hepcidin-20, cynomolgus hepcidin-25 and marmoset hepcidin-25.

It is to be acknowledged that whenever it is referred herein to hepcidin, such hepcidin is hepcidin-25, if not indicated to the contrary.

The different types of hepcidin binding nucleic acid molecules that bind to hepcidin comprise three different stretches of nucleotides: the first terminal stretch of nucleotides, the central stretch of nucleotides and second terminal stretch of nucleotides. In general, hepcidin binding nucleic acid molecules of the present invention comprise at the 5′-end and the 3′-end the terminal stretches of nucleotides: the first terminal stretch of nucleotides and the second terminal stretch of nucleotides (also referred to as 5′-terminal stretch of nucleotides and 3′-terminal stretch of nucleotides). The first terminal stretch of nucleotides and the second terminal stretch of nucleotides can, in principle due to their base complementarity, hybridize to each other, whereby upon hybridization a double-stranded structure is formed. However, such hybridization is not necessarily realized in the molecule under physiological and/or non-physiological conditions. The three stretches of nucleotides of hepcidin binding nucleic acid molecules—the first terminal stretch of nucleotides, the central stretch of nucleotides and second terminal stretch of nucleotides—are arranged to each other in 5′→3′-direction; the first terminal stretch of nucleotides—the central stretch of nucleotides—the second terminal stretch of nucleotides. However, alternatively, the second terminal stretch of nucleotides, the central stretch of nucleotides and the terminal first stretch of nucleotides are arranged to each other in 5′→3′-direction.

The differences in the sequences of the defined boxes or stretches between the different hepcidin binding nucleic acid molecules influence the binding affinity to hepcidin. Based on binding analysis of the different hepcidin binding nucleic acid molecules of the present invention the central stretch and the nucleotides forming the same are individually and more preferably in their entirety essential for binding to hepcidin.

The terms ‘stretch’ and ‘stretch of nucleotide’ are used herein in a synonymous manner if not indicated to the contrary.

In a preferred embodiment the nucleic acid according to the present invention is a single nucleic acid molecule. In a further embodiment, the single nucleic acid molecule is present as a multitude of the single nucleic acid molecule or as a multitude of the single nucleic acid molecule species.

It will be acknowledged by the ones skilled in the art that the nucleic acid molecule in accordance with the invention preferably consists of nucleotides which are covalently linked to each other, preferably through phosphodiester links or linkages.

It is within the present invention that the nucleic acids according to the present invention comprise two or more stretches or part(s) thereof can, in principle, hybridise with each other.

Upon such hybridisation a double-stranded structure is formed. It will be acknowledged by the ones skilled in the art that such hybridisation may or may not occur, particularly under in vitro and/or in vivo conditions. Also, in case of such hybridisation, it is not necessarily the case that the hybridisation occurs over the entire length of the two stretches where, at least based on the rules for base pairing, such hybridisation and thus formation of a double-stranded structure may, in principle, occur. As preferably used herein, a double-stranded structure is a part of a nucleic acid molecule or a structure formed by two or more separate strands or two spatially separated stretches of a single strand of a nucleic acid molecule, whereby at least one, preferably two or more base pairs exist which are base pairing preferably in accordance with the Watson-Crick base pairing rules. It will also be acknowledged by the one skilled in the art that other base pairing such as Hoogsten base pairing may exist in or form such double-stranded structure. It is also to be acknowledged that the feature that two stretches hybridize preferably indicates that such hybridization is assumed to happen due to base complementarity of the two stretches.

In a preferred embodiment the term arrangement as used herein, means the order or sequence of structural or functional features or elements described herein in connection with the nucleic acids disclosed herein.

It will be acknowledged by the person skilled in the art that the nucleic acids according to the present invention are capable of binding to hepcidin. Without wishing to be bound by any theory, the present inventors assume that the hepcidin binding results from a combination of three-dimensional structural traits or elements of the claimed nucleic acid molecule, which are caused by orientation and folding patterns of the primary sequence of nucleotides forming such traits or elements. It is evident that the individual trait or element may be formed by various different individual sequences the degree of variation of which may vary depending on the three-dimensional structure such element or trait has to form. The overall binding characteristic of the claimed nucleic acid results from the interplay of the various elements and traits, respectively, which ultimately results in the interaction of the claimed nucleic acid with its target, i. e. hepcidin. Again without being wished to be bound by any theory, the central stretch that is characteristic for Type B and Type C hepcidin binding nucleic acids, and the first stretch Box A and the second stretch Box B that are characteristic for Type A hepcidin binding nucleic acids, seem to be important for mediating the binding of the claimed nucleic acid with hepcidin. Accordingly, the nucleic acids according to the present invention are suitable for the interaction with and detection of hepcidin. Also, it will be acknowledged by the person skilled in the art that the nucleic acids according to the present invention are antagonists to hepcidin. Because of this the nucleic acids according to the present invention are suitable for the treatment and prevention, respectively, of any disease or condition which is associated with or caused by hepcidin. Such diseases and conditions may be taken from the prior art which establishes that hepcidin is involved or associated with said diseases and conditions, respectively, and which is incorporated herein by reference providing the scientific rational for the therapeutic and diagnostic use of the nucleic acids according to the invention.

It is within the present invention that the nucleic acid according to the present invention is a nucleic acid molecule. Insofar the terms nucleic and nucleic acid molecule are used herein in a synonymous manner if not indicated to the contrary. In one embodiment of the present application the nucleic acid and thus the nucleic acid molecule comprises a nucleic acid molecule which is characterized in that all of the consecutive nucleotides forming the nucleic acid molecule are linked with or connected to each other by one or more than one covalent bond. More specifically, each of such nucleotides is linked with or connected to two other nucleotides, preferably through phosphodiester bonds or other bonds, forming a stretch of consecutive nucleotides. In such arrangement, however, the two terminal nucleotides, i.e. preferably the nucleotide at the 5′ end and at the 3′ end, are each linked to a single nucleotide only under the proviso that such arrangement is a linear and not a circular arrangement and thus a linear rather than a circular molecule.

In another embodiment of the present application the nucleic acid and thus the nucleic acid molecule comprises at least two groups of consecutive nucleotides, whereby within each group of consecutive nucleotides each nucleotide is linked with or connected to two other nucleotides, preferably through phosphodiester bonds or other bonds, forming a stretch of consecutive nucleotides. In such arrangement, however, the two terminal nucleotides, i.e. preferably the nucleotide at the 5′ end and at the 3′ end, of each of said at least two groups of consecutive nucleotides are each linked to a single nucleotide only. In such embodiment, the two groups of consecutive nucleotides, however, are not linked with or connected to each other through a covalent bond which links one nucleotide of one group and one nucleotide of another or the other group through a covalent bond, preferably a convalent bond formed between a sugar moiety of one of said two nucleotides and a phosphor moiety of the other of said two nucleotides or nucleosides. In an alternative embodiment, the two groups of consecutive nucleotides, however, are linked with or connected to each other through a covalent bond which links one nucleotide of one group and one nucleotide of another or the other group through a convalent bond, preferably a convalent bond formed between a sugar moiety of one of said two nucleotides and a phosphor moiety of the other of said two nucleotides or nucleosides. Preferably, the at least two groups of consecutive nucleotides are not linked through any covalent bond. In another preferred embodiment, the at least two groups are linked through a covalent bond which is different from a phosphodiester bond. In still another embodiment, the at least two groups are linked through a covalent bond which is a phosphodiester bond. Furthermore, preferably, the two groups of consecutive nucleotides are linked or connected to each other through a covalent bond whereby the covalent bond is formed between the nucleotide at the 3′-end of the first of the two groups of consecutive nucleotides and the nucleotide at the 5′-end of the second of the two groups of consecutive nucleotides or the covalent bond is formed between the nucleotide at the 5′-end of the first of the two groups of consecutive nucleotides and the nucleotide at the 3′- end of the second of the two groups of consecutive nucleotides.

The nucleic acids according to the present invention shall also comprise nucleic acids which are essentially homologous to the particular sequences disclosed herein. The term substantially homologous shall be understood such as the homology is at least 75%, preferably 85%, more preferably 90%, and most preferably more that 95%, 96%, 97%, 98% or 99%.

The actual percentage of homologous nucleotides present in the nucleic acid according to the present invention will depend on the total number of nucleotides present in the nucleic acid. The percent modification can be based upon the total number of nucleotides present in the nucleic acid.

The homology between two nucleic acid molecules can be determined as known to the person skilled in the art. More specifically, a sequence comparison algorithm may be sued for calculating the percent sequence homology for the test sequence(s) relative to the reference sequence, based on the designated program parameters. The test sequence is preferably the sequence or nucleic acid molecule which is said to be homologous or to be tested whether it is homologous, and if so, to what extent, to a different nucleic acid molecule, whereby such different nucleic acid molecule is also referred to as the reference sequence. In an embodiment, the reference sequence is a nucleic acid molecule as described herein, preferably a nucleic acid molecule having a sequence according to any one of SEQ.ID.No. 29 to 43, SEQ.ID.No. 45 to 48, SEQ.ID.No. 110 to 156, SEQ.ID.No. 158 to 176 or SEQ.ID.No. 179 to 181. Optimal alignment of sequences for comparison can be conducted, e.g., by the local homology algorithm of Smith & Waterman (Smith & Waterman, 1981) by the homology alignment algorithm of Needleman & Wunsch (Needleman & Wunsch, 1970) by the search for similarity method of Pearson & Lipman (Pearson & Lipman, 1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity is the algorithm used in the basic local alignment search tool (hereinafter “BLAST”), see, e.g. Altschul et al (Altschul et al. 1990 and Altschul et al, 1997). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al (McGinnis et al, 2004).

The nucleic acids according to the present invention shall also comprise nucleic acids which have a certain degree of identity relative to the nucleic acids disclosed herein and defined by their nucleotide sequence. More preferably, the instant invention also comprises those nucleic acid molecules which have an identity of at least 75%, preferably 85%, more preferably 90%, and most preferably more than 95%, 96%, 97%, 98% or 99% relative to the nucleic acids disclosed herein and defined by their nucleotide sequence or a part thereof.

The term inventive nucleic acid or nucleic acid according to the (present) invention, whereby both terms are used in an interchangeable manner, shall also comprise those nucleic acids comprising the nucleic acids sequences disclosed herein or part thereof, preferably to the extent that the nucleic acids or said parts are involved in the binding to human hepcidin. Such nucleic acid is, in an embodiment, one of the nucleic acid molecules described herein, or a derivative and/or a metabolite thereof, whereby such derivative and/or metabolite are preferably a truncated nucleic acid compared to the nucleic acid molecules described herein. Truncation may be related to either or both of the ends of the nucleic acids as disclosed herein. Also, truncation may be related to the inner sequence of nucleotides of the nucleic acid, i.e. it may be related to the nucleotide(s) between the 5′ and the 3′ terminal nucleotide, respectively. Moreover, truncation shall comprise the deletion of as little as a single nucleotide from the sequence of the nucleic acids disclosed herein. Truncation may also be related to more than one stretch of the inventive nucleic acid(s), whereby the stretch can be as little as one nucleotide long. The binding of a nucleic acid according to the present invention can be determined by the ones skilled in the art using routine experiments or by using or adopting a method as described herein, preferably as described herein in the example part.

The nucleic acids according to the present invention may be either D-nucleic acids or L-nucleic acids. Preferably, the inventive nucleic acids are L-nucleic acids. In addition it is possible that one or several parts of the nucleic acid are present as D-nucleic acids or at least one or several parts of the nucleic acids are L-nucleic acids. The term “part” of the nucleic acids shall mean as little as one nucleotide. Therefore, in a particularly preferred embodiment, the nucleic acids according to the present invention consist of L-nucleotides and comprise at least one D-nucleotide. Such D-nucleotide is preferably attached to a part different from the stretches defining the nucleic acids according to the present invention, preferably those parts thereof, where an interaction and with other parts of the nucleic acid or with the target, i.e. hepcidin, is involved. Preferably, such D-nucleotide is attached at a terminus of any of the stretches or at a terminus of any nucleic acid according to the present invention, respectively. In a further preferred embodiment, such D-nucleotides may act as a spacer or a linker, preferably attaching modifications or modification groups, such as PEG and HES to the nucleic acids according to the present invention.

It is also within an embodiment of the present invention that each and any of the nucleic acid molecules described herein in their entirety in terms of their nucleic acid sequence(s) are limited to the particular nucleotide sequence(s). In other words, the terms “comprising” or “comprise(s)” shall be interpreted in such embodiment in the meaning of containing or consisting of.

It is also within the present invention that the nucleic acids according to the present invention are part of a longer nucleic acid whereby this longer nucleic acid comprises several parts whereby at least one such part is a nucleic acid according to the present invention, or a part thereof. The other part(s) of these longer nucleic acids can be either one or several D-nucleic acid(s) or one or several L-nucleic acid(s). Any combination may be sued in connection with the present invention. These other part(s) of the longer nucleic acid either alone or taken together, either in their entirety or in a particular combination, can exhibit a function which is different from binding, preferably from binding to hepcidin. One possible function is to allow interaction with other molecules, whereby such other molecules preferably are different from hepcidin, such as, e.g., for immobilization, cross-linking, detection or amplification. In a further embodiment of the present invention the nucleic acids according to the invention comprise, as individual or combined moieties, several of the nucleic acids of the present invention. Such nucleic acids comprising several of the nucleic acids of the present invention is also encompassed by the term longer nucleic acid.

L-nucleic acids or L-nucleic acid molecules as used herein are nucleic acids or nucleic acid molecules consisting of L-nucleotides, preferably consisting completely of L-nucleotides.

D-nucleic acids or D-nucleic acid molecules as used herein are nucleic acids or nucleic acid molecules consisting of D-nucleotides, preferably consisting completely of D-nucleotides.

Also, if not indicated to the contrary, any nucleotide sequence is set forth herein in 5′→3′ direction.

As preferably used herein any position of a nucleotide is determined or referred to relative to the 5′ end of a sequence, a stretch or a substretch. Accordingly, a second nucleotide is the second nucleotide counted from the 5′ end of the sequence, stretch and substretch, respectively. Also, in accordance therewith, a punultimate nucleotide is the second nucleotide counted from the 3′ end of a sequence, stretch and substretch, respectively.

Irrespective of whether the inventive nucleic acid consists of D-nucleotides, L-nucleotides or a combination of both with the combination being e.g. a random combination or a defined. sequence of stretches consisting of at least one L-nucleotide and at least one D-nucleic acid, the nucleic acid may consist of desoxyribonucleotide(s), ribonucleotide(s) or combination thereof.

Designing the inventive nucleic acids as L-nucleic acids is advantageous for several reasons. L-nucleic acids are enantiomers of naturally occurring nucleic acids. D-nucleic acids, however, are not very stable in aqueous solutions and particularly in biological systems or biological samples due to the widespread presence of nucleases. Naturally occurring nucleases, particularly nucleases from animal cells are not capable of degrading L-nucleic acids. Because of this the biological half-life of the L-nucleic acid is significantly increased in such a system, including the animal and human body. Due to the lacking degradability of L-nucleic acids no nuclease degradation products are generated and thus no side effects arising therefrom observed. This aspect delimits the L-nucleic acids of factually all other compounds which are used in the therapy of diseases and/or disorders involving the presence of hepcidin, L-nucleic acids which specifically bind to a target molecule through a mechanism different from Watson Crick base pairing, or aptamers which consists partially or completely of L-nucleotides, particularly with those parts of the aptamer being involved in the binding of the aptamer to the target molecule, are also called Spiegelmers. Aptamers as such are known to a person skilled in the art and are, among others, described in ‘The Aptamer Handbook’ (eds. Klussmann, 2006).

It is also within the present invention that the nucleic acids according to the invention, regardless whether they are present as D-nucleic acids, L-nucleic acids or D, L-nucleic acids or whether they are DNA or RNA, may be present as single-stranded or double-stranded nucleic acids. Typically, the inventive nucleic acids are single-stranded or double-stranded nucleic acids. Typically, the inventive nucleic acids are single-stranded nucleic acids which exhibit defined secondary structures due to the primary sequence and may thus also form tertiary structures. The inventive nucleic acids, however, may also be double-stranded in the meaning that two strands regardless whether they are two separate strands or whether they are bound, preferably covalently, to each other, which are complementary or partially complementary to each other are hybridised to each other.

The inventive nucleic acids may be modified. Such modifications may be related to the single nucleotide of the nucleic acid and are well known in the art. Examples for such modification are described by, among others, Venkatesan et al. (Venkatesan, Kim et al. 2003) and Kusser (Kusser, 2000). Such modification can be a H atom, a F atom or O—CH3 group or NH2-group at the 2′ position of the individual nucleotide of which the nucleic acid consists. Also, the nucleic acid according to the present invention can comprise at least one LNA nucleotide. In an embodiment the nucleic acid according to the present invention consists of LNA nucleotides.

In an embodiment, the nucleic acids according to the present invention may be a multipartite nucleic acid. A multipartite nucleic acid as used herein, is a nucleic acid which consists of at least two separate nucleic acid strands. These at least two nucleic acid strands form a functional unit whereby the functional unit is a ligand to a target molecule. The at least two nucleic acid strands may be derived from any of the inventive nucleic acids by either cleaving the nucleic acid molecule to generate two strands or by synthesising one nucleic acid corresponding to a first part of the inventive, i.e. overall nucleic acid and another nucleic acid corresponding to the second part of the overall nucleic acid. It is to be acknowledged that both the cleavage and the synthesis may be applied to generate a multipartite nucleic acid where there are more than two strands as exemplified above. In other words, the at least two separate nucleic acid strands are typically different from two strands being complementary and hybridising to each other although a certain extent of complementarity may result in the hybridisation of said separate strands.

Finally it is also within the present invention that a fully closed, i.e. circular structure for the nucleic acids according to the present invention is realized, i.e. that the nucleic acids according to the present invention are closed in an embodiment, preferably through a covalent linkage, whereby more preferably such covalent linkage is made between the 5′ end and the 3′ end of the nucleic acid sequences as disclosed herein or any derivative thereof.

A possibility to determine the binding constants of the nucleic acid molecules according to the present invention is the use of surface plasmon resonance as described in example 4 which confirms the above finding that the nucleic acids according to the present invention exhibit a favourable K_{D} value range. An appropriate measure in order to express the intensity of the binding between the individual nucleic acid molecule and the target which is in the present case hepcidin, is the so-called K_{D} value which as such as well the method for its determination are known to the one skilled in the art.

Preferably, the K_{D} value shown by the nucleic acids according to the present invention is below 1 μM. A K_{D} value of about 1 μM is said to be characteristic for a non-specific binding of a nucleic acid to a target. As will be acknowledged by the ones skilled in the art, the K_{D} value of a group of compounds such as the nucleic acids according to the present invention is within a certain range. The above-mentioned K_{D} of about 1 μM is a preferred upper limit for the K_{D} value. The lower limit for the K_{D} of target binding nucleic acids can be as little as about 10 picomolar or can be higher. It is within the present invention that the K_{D} values of individual nucleic acids binding to hepcidin is preferably within this range. Preferred ranges can be defined by choosing any first number within this range and any second number within this range. Preferred upper K_{D} values are 250 nM and 100 nM, preferred lower K_{D} values are 50 nM, 10 nM, 1 nM, 100 pM and 10 pM. The more preferred upper K_{D} value is 2.5 nM, the more preferred lower K_{D} value is 400 pM.

In addition to the binding properties of the nucleic acid molecules according to the present invention, the nucleic acid molecules according to the present invention inhibit the function of the respective target molecule which is in the present case hepcidin. The inhibition of the function of hepcidin—for instance the stimulation of the respective receptors as described previously—is achieved by binding of nucleic acid molecules according to the present invention to hepcidin and forming a complex of a nucleic acid molecule according to the present invention and hepcidin. Such complex of a nucleic acid molecule and hepcidin cannot stimulate the receptors that normally are stimulated by hepcidin. Accordingly, the inhibition of receptor function by nucleic acid molecules according to the present invention is independent from the respective receptor that can be stimulated by hepcidin but results from preventing the stimulation of the receptor by hepcidin by the nucleic acid molecules according to the present invention.

A possibility to determine the inhibitory constant of the nucleic acid molecules according to the present invention is the use of the methods as described in example 5 which confirms the above finding that the nucleic acids according to the present invention exhibit a favourable inhibitory constant which allows the use of said nucleic acids in a therapeutic treatment scheme. An appropriate measure in order to express the intensity of the inhibitory effect of the individual nucleic acid molecule on interaction of the target which is in the present case hepcidin and the respective receptor, is the so-called half maximal inhibitory concentration (abbr. IC₅₀) which as such as well the method for its determination are known to the one skilled in the art.

Preferably, the IC₅₀ value shown by the nucleic acid molecules according to the present invention is below 1 μM. An IC₅₀ value of about 1 μM is said to be characteristic for a non-specific inhibition of target functions by a nucleic acid molecule. As will be acknowledged by the ones skilled in the art, the IC₅₀ value of a group of compound such as the nucleic acid molecules according to the present invention is within a certain range. The above-mentioned IC₅₀ of about 1 μM is a preferred upper limit for the IC₅₀ value. The lower limit for the IC₅₀ of target binding nucleic acid molecules can be as little as about 10 picomolar or can be higher. It is within the present invention that the IC₅₀ values of individual nucleic acids binding to hepcidin is preferably within this range. Preferred ranges can be defined by choosing any first number within this range and any second number within this range. Preferred upper IC₅₀ values are 250 nM and 100 nM, preferred lower IC₅₀ values are 50 nM, 10 nM, 1 nM, 100 pM and 10 pM. The more preferred upper IC₅₀ value is 5 nm, the more preferred lower IC₅₀ value is 1 nM.

The nucleic acid molecules according to the present invention may have nay length provided that they are still able to bind to the target molecule. It will be acknowledged by a person skilled in the art that there are preferred lengths for the nucleic acids according to the present inventions. Typically, the length is between 15 and 120 nucleotides. It will be acknowledged by the ones skilled in the art that any integer between 15 and 120 is a possible length for the nucleic acids according to the present invention. More preferred ranges for the length of the nucleic acids according to the present invention are lengths of about 20 to 100 nucleotides, about 20 to 80 nucleotides, about 20 to 60 nucleotides, about 20 to 50 nucleotides and about 30 to 50 nucleotides.

It is within the present invention that the nucleic acids disclosed herein comprise a moiety which preferably is a high molecular weight moiety and/or which preferably allows to modify the characteristics of the nucleic acid in terms of, among others, residence time in an animal body, preferably a human body. A particularly preferred embodiment of such modification is PEGylation and HESylation of the nucleic acids according to the present invention. As used herein PEG stands for poly(ethylene glycole) and HES for hydroxyethyl starch. PEGylation as preferably used herein is the modification of a nucleic acid according to the present invention whereby such modification consists of a PEG moiety which is attached to a nucleic acid according to the present invention. HESylation as preferably used herein is the modification of a nucleic acid according to the present invention whereby such modification consists of a HES moiety which is attached to a nucleic acid according to the present invention. The modifications such as linear poly(ethylene)glycol, branched poly(ethylene)glycol, hydroxyethyl starch, a peptide, a protein, a polysaccharide, a sterol, polyoxypropylene, polyoxyamidate, poly(2-hydroxyethyl)-L-glutamine and polyethylene glycol as well as the process of modifying a nucleic acid using such modifications are described in European patent application EP 1 306 382, the disclosure of which is herewith incorporated in its entirety by reference.

Preferably, the molecular weight of a modification consisting of or comprising a high molecular weight moiety is about from 2,000 to 250,000 Da, preferably 20,000 to 200,000 Da. In the case of PEG being such high molecular weight moiety the molecular weight is preferably 20,000 to 120,000 Da, more preferably 40,000 to 80,000 Da. In the case of HES being such high molecular weight moiety the molecular weight is preferably 20,000 to 200,000 Da, more preferably 40,000 to 150,000 Da. The process of HES modification is, e.g., described in German patent application DE 1 2004 006 249.8 the disclosure of which is herewith incorporated in its entirety by reference.

It is within the present invention that either of PEG and HES may be used as either a linear or branched form as further described in patent applications WO2005/074993 WO2003/035665 and EP1496076. Such modification can, in principle, be made to the nucleic acid molecules of the present invention at any position thereof. Preferably such modification is made either to the 5′-terminal nucleotide, the 3′-terminal nucleotide and/or any nucleotide between the 5′ nucleotide and the 3′ nucleotide of the nucleic acid molecule according to the invention.

The modification and preferably the PEG and/or HES moiety can be attached to the nucleic acid molecule of the present invention either directly or indirectly, preferably through a linker. It is also within the present invention that the nucleic acid molecule according to the present invention comprises one or more modifications, preferably one or more PEG and/or HES moiety. In an embodiment the individual linker molecule attaches more than one PEG moiety or HES moiety to a nucleic acid molecule according to the present invention. The linker used in connection with the present invention can itself be either linear or branched. This kind of linkers are known to the ones skilled in the art and are further described in patent applications WO2005/074993, WO2003/035665 and EP1496076.

In a preferred embodiment the linker is a biodegradable linker. The biodegradable linker allows to modify the characteristics of the nucleic acid according to the present invention in terms of, among other, residence time in an animal body, preferably in a human body, due to release of the modification from the nucleic acid according to the present invention. Usage of a biodegradable linker may allow a better control of the residence time of the nucleic acid according to the present invention. A preferred embodiment of such biodegradable linker is a biodegradable linker as described in, but not limited to, international patent applications WO2006/052790, WO2008/034122, WO2004/092191 and WO2005/099768.

In one embodiment the linker is a linker that comprise an amino group and two hexaethylene glycol (abbr. HEG) moieties with the following structure: Amino-HEG-HEG. In a preferred embodiment the linker is conjugated to the 5′- or 3′-end of a nucleic acid molecule according to the present invention, more preferably to the 5′-end of a nucleic acid molecule according to the present invention leading to the following structure; 5′-Amino-HEG-HEG-5′-terminal nucleotide of the nucleic acid molecule according to the present invention.

It is within the present invention that the modification or modification group is a biodegradable modification, whereby the biodegradable modification can be attached to the nucleic acid molecule of the present invention either directly or indirectly, preferably through a linker. The biodegradable modification allows to modify the characteristics of the nucleic acid according to the present invention in terms of, among other, residence time in an animal body, preferably in a human body, due to release or degradation of the modification from the nucleic acid according to the present invention. Usage of biodegradable modification may allow a better control of the residence time of the nucleic acid according to the present invention. A preferred embodiment of such biodegradable modification is biodegradable as described in, but not restricted to, international patent applications WO2002/065963, WO2003/070823, WO2004/113394 and WO2000/41647, preferably in WO2000/41647, page 18, line 4 to 24.

Beside the modifications as described above, other modifications can be used to modify the characteristics of the nucleic acids according to the present invention, whereby such other modifications may be selected from the group of proteins, lipids such as cholesterol and sugar chains such as amylase, dextran etc.

Without wishing to be bound by an theory, it seems that by modifying the nucleic acids according to the present invention with high molecular weight moiety such as a polymer and more particularly one or several of the polymers disclosed herein, which are preferably physiologically acceptable, the excretion kinetic is changed. More particularly, it seems that due to the increased molecular weight of such modified inventive nucleic acids and due to the nucleic acids of the invention not being subject to metabolism particularly when in the L form, excretion from an animal body, preferably from a mammalian body and more preferably from a human body is decreased. As excretion typically occurs via the kidneys, the present inventors assume that the glomerular filtration rate of the thus modified nucleic acids is significantly reduced compared to the nucleic acids not having this kind of high molecular weight modification which results in an increase in the residence time in the animal body. In connection therewith it is particularly noteworthy that, despite such high molecular weight modification the specificity of the nucleic acids according to the present invention is not affected in a detrimental manner. Insofar, the nucleic acids according to the present invention have among others, the surprising characteristics—which normally cannot be expected from pharmaceutically active compounds—such that a pharmaceutical formulation providing for a sustained release is not necessarily required to provide for a sustained release of the nucleic acids according to the present invention. Rather the nucleic acids according to the present invention in their modified form comprising a high molecular weight moiety, can as such already be used as a sustained release-formulation as they act, due to their modification, already as if they were released from a sustained-release formulation. Insofar, the modification(s) of the nucleic acid molecules according to the present invention as disclosed herein and the thus modified nucleic acid molecules according to the present invention and any composition comprising the same may provide for a distinct, preferably controlled pharmacokinetics and biodistribution thereof. This also includes residence time in circulation and distribution to tissues. Such modifications are further described in the patent application WO2003/035665.

The renal excretion of thus modified nucleic acid molecules according to the present invention—preferably PEG or HES—modified nucleic acid molecules—is decelerated in comparison to an non-modified nucleic acids because of the high molecular weight moiety, preferably a PEG or HES moiety. A more decelerated renal excretion of thus modified nucleic acid molecules according to the present invention is more likely if the patient has impaired kidney function.

Thus, it is also within the present invention that the nucleic acids according to the present invention do not comprise any modification and particularly no high molecular weight modification such as PEG or HES.

The present invention solves the problem underlying the present invention by a method for reducing the level of hepcidin in a body fluid from a subject, comprising
a) providing a nucleic acid molecule capable of binding to hepcidin,
b) bringing the nucleic acid molecule into contact with a body fluid under conditions that allow for the binding of hepcidin to the nucleic acid molecule, thereby forming a complex of hepcidin and the nucleic acid molecule, and
c) removing the complex from the body fluid or removing the hepcidin from the body fluid.

In a first preferred embodiment of the present invention the hepcidin binding nucleic acid is immobilised on a support, whereby preferably the support with the hepcidin binding nucleic acid immobilised thereto. Said support is located ex vivo, e.g. in a medical device, preferably in a medical device for apheresis. In such medical device the body fluid comprising hepcidin is in contact with the support to form the complex of hepcidin and the hepcidin binding nucleic acid. Due to the immobilised hepcidin binding nucleic acid hepcidin can be removed from the body fluid.

In a second preferred embodiment of the present invention the hepcidin binding nucleic acid comprises a modification that allows the immobilisation of such modified hepcidin binding nucleic acid by affinity immobilisation, e.g. by a ligand that binds to the modification and is linked to the support. Using such modified hepcidin binding nucleic acid and a ligand immobilised on a support allows the use of such modified hepcidin binding nucleic acid in vivo (in the body fluid). In the body fluid the modified hepcidin binding nucleic acid binds to hepcidin and inhibits its function in vivo. The complex of hepcidin and the modified hepcidin binding nucleic acid is removed from the body by affinity immobilisation of the modification by the ligand linked the support. Said support is located ex vivo, e.g. in a medical device, preferably in medical device for apheresis.

In a third preferred embodiment of the present invention the body fluid is brought into contact with a semi-permeable membrane, whereby the body fluid is on the one side of the membrane and dialysate is the other side of said membrane, such that hepcidin and/or the complex of hepcidin and the hepcidin nucleic binding acid diffuses through the semi-permeable membrane from the body fluid to the dialysate. In this case the hepcidin binding nucleic acid is non-modified. Using such a method allows the use of a hepcidin binding nucleic acid in vivo (in the body fluid). In the body fluid the hepcidin binding nucleic acid binds to hepcidin and inhibits its function in vivo. The complex of hepcidin and the hepcidin binding nucleic acid is removed from the body. Said support is located ex vivo, e.g. in a medical device, preferably in a medical device for dialysis.

In a fourth preferred embodiment of the present invention the body fluid is brought into contact with a semi-permeable membrane, whereby the body fluid is on the one side of the membrane and dialysate is the other side of said membrane. In this case the hepcidin binding nucleic acid is modified, whereby modification is preferably a high molecular weight modification. Due to the size of the high molecular weight modification such modified hepcidin binding nucleic acid can not cross the semi-permeable membrane. Therefore the modified hepcidin binding nucleic acid is in the dialysate, hepcidin cross the semi-permeable membrane, and binds to the modified hepcidin binding nucleic acid in the dialysate. Thereby the complex of hepcidin and the hepcidin binding nucleic acid is removed from the body. Said support is located ex vivo, e.g. in a medical device, preferably in a medical device for dialysis.

The term apheresis refers to a procedure in which blood is removed from a subject and passed through a device, which separates out a specific component whereas the rest of the components are returned to the subject. As known to a person of skill in the art, apheresis has been used for depletion of disease causing biomolecules through interaction of the biomolecule with an immobilised ligand thereof, for example, antibody-antigen interactions. That is, the ligand is immobilized on an adsorber column and blood removed using an apheresis device is circulated through the column. The disease-inducing biomolecule present in the blood binds specifically to the immobilized ligand and is retained while the rest of the blood is returned to the subject.

Terman et al. (U.S. Pat. No. 4,215,688) describes such a method and device for the ex vivo treatment of disease. The blood is withdrawn from a subject, plasma is separated from the blood and passed through a cylinder containing beads to which an immuno-absorbent agent is immobilized. The treated plasma is recombined with the blood and returned to the subject. Similarly, U.S. Pat. No. 4,685,900 describes a system where body fluids are passed through a chamber containing a biospecific polymer grafted onto and extending away from a support via a spacer moiety. The biospecific polymer, e.g., a hydrogel chemically binds to specific pathological effectors and thereby removes the pathological effectors from the body fluid. At the end of the treatment, the body fluid is returned to the subject. Examples of moieties used as immobilised ligands in an apheresis procedure are antibodies and antigens (see WO05107802 and U.S. Pat. No. 4,375,414), microbial ligands (U.S. Pat. No. 4,614,513), biospecific polymers, e.g., hydrogel (see above), and molecular imprint material (see WO06017763).

The first and second preferred embodiment of the present invention is to provide an hepcidin binding nucleic acid immobilized on a solid support, e.g. as it is known for use in apheresis. In particular, it is to provide a hepcidin binding nucleic acid, which when for example bound to a solid support is surprisingly stable and retains its functionality. By functionality, it is meant that the hepcidin binding nucleic acid is able to form distinct two-dimensional and/or three-dimensional structures and thereby bind with high affinity and specificity to the target molecule hepcidin. Using an apheresis device the blood is removed form a subject and is circulated through a column containing a support where the hepcidin binding nucleic acid has been immobilized. The treated blood is returned to the subject. It is preferred that plasma is separated from the blood and circulated through a column containing a solid support where hepcidin binding nucleic acids have been immobilized. The treated plasma is recombined with the blood and returned to the subject. It is further preferred that fibrinogen, clotting factors and cells are separated out to obtain serum from the blood. The serum is circulated through a column containing a solid support where hepcidin binding nucleic acids have been immobilized. The serum is recombined with the blood and returned to the subject.

The advantages of such methods are several. Firstly, the method is highly specific. Only the disease-inducing biomolecule, in particular hepcidin, is targeted. Secondly, the method is convenient as the method of apheresis is a routine procedure conducted in laboratories.

During a dialysis procedure blood flows on one side of a semi-permeable membrane while a dialysate or special dialysis fluid flows on the other side of the membrane. As known in the art, the semi-permeable membrane is composed of a thin material with various sized pores allowing only solutes and small molecules to flow through but preventing larger molecules and cells from crossing over. Suitable dialysates are known to persons skilled in the art. The principle behind the dialysis procedure is the diffusion of solutes across a semi-permeable membrane. A dialysis device is used in subjects who are experiencing renal failure. Waste products and water typically excreted by a healthy kidney are removed through a dialysis device. Forms of dialysis include hemodialysis, peritoneal dialysis and a related procedure, hemofiltration and hemodiafiltration.

Hemodialysis removes solutes and water by circulating blood outside the body through an external filter, called a dialyzer, that contains a semipermeable membrane. The blood flows in one direction and the dialysate flows in the opposite. the counter-current flow of the blood and dialysate maximizes the concentration gradient of solutes between the blood and dialysate, which helps to remove the solutes from the blood. The concentrations of solutes are undesirably high in the blood, but low or absent in the dialysis solution and constant replacement of the dialysate ensures that the concentration of undesired solutes is kept low on this side of the membrane. The dialysis solution has levels of minerals like potassium and calcium that are similar to their natural concentration in healthy blood. Ultrafiltration occurs by increasing the hydrostatic pressure across the dialyzer membrane. This usually is done by applying a negative pressure to the dialysate compartment of the dialyzer. This pressure gradient causes water and dissolved solutes to move from blood to dialysate, and allows the removal of several litres of excess fluid during a typical 3 to 5 hour treatment.

Hemofiltration is a similar treatment to hemodialysis, but it makes use of a different principle. The blood is pumped through a dialyzer or “hemofilter” as in dialysis, but no dialysate is used. A pressure gradient is applied; as a result, water moves across the very permeable membrane rapidly, “dragging” along with it many dissolved substances, importantly ones with large molecular weights, which are cleared less well by hemodialysis. Salts and water lost from the blood during this process are replaced with a “substitution fluid” that is infused into the extracorporeal circuit during the treatment.

Hemodiafiltration is a term used to describe several methods of combining hemodialysis and hemofiltration in one process. Blood is pumped through the blood compartment of a high fllux dialyzer, and a high rate of ultrafiltration is used, so there is a high rate of movement of water and solutes from blood to dialysate that must be replaced by substitution fluid that is infused directly into the blood line. However, dialysis solution is also run through the dialysate compartment of the dialyzer. The combination is theoretically useful because it results in good removal of both large and small molecular weight solutes.

In the third embodiment of the invention the hepcidin binding nucleic acid is administered to the subject prior to dialysis and blocks the activity of hepcidin. In the case of a hepcidin binding nucleic acid not modified with a high molecular weight moiety, during the dialysis procedure the complex of hepcidin and the hepcidin binding nucleic acid traverses the appropriate semi-permeable membrane, as known to a person of skill in the art, from an area of high concentration to an area of low concentration of these molecules. The final result is reduced concentration of hepcidin in the blood of the subject. Is the hepcidin binding nucleic acid modified with a high molecular weight moiety, the high molecular weight moiety has to be removed before or within the dialysis procedure in order to facilitate the transit of the non-modified hepcidin binding nucleic acid through the dialysis membrane. The removal of the high molecular weight moiety can be realized by a cleavable linker between the nucleotides of the hepcidin binding nucleic acid and the high molecular weight moiety or by cleavage of the hepcidin binding nucleic acid at the end at which the high molecular weight moiety is linked to. Such cleavage can be done by a nucleic acid molecule specific nucleic acid based enzyme, preferably an RNA or DNA enzyme.

According to the fourth embodiment of the invention, a suitable dialysate (as e.g. used in a dialysis method) would additionally contain a hepcidin binding nucleic acid modified with a high molecular weight moiety, preferably the high molecular weight moiety is selected from the group of PEG and HES. As hepcidin diffuses through the semi-permeable membrane to the dialysate, it forms a complex with the hepcidin binding nucleic acid. this complex is too large to flow back across the membrane. As a result, hepcidin levels are progressively reduced during the dialysis procedure.

the term immobilization means binding a compound, preferably a hepcidin binding nucleic acid, to a support.

The hepcidin binding nucleic acid is bound or immobilized to a support either via a 3′ end or 5′ end of the hepcidin binding nucleic acid. The hepcidin binding nucleic acid is immobilized either directly or indirectly using a ligand. The immobilization of the hepcidin binding hepcidin binding nucleic acid to a support according to the invention can be selected from the group comprising covalent bonds, non-covalent bonds, hydrogen bonds, van der Waals interactions, coulombic interactions and/or hydrophobic interactions, coordinate bonds and combinations thereof as required in connection with the various types of immobilization.

In one embodiment of the invention, the support to which the hepcidin binding nucleic acid is immobilized is solid phase, matrix or solid support.

The term conjugation means a covalent bond between the hepcidin binding nucleic acid and the support.

In a preferred embodiment of the immobilized hepcidin binding nucleic acid, the solid phase, matrix or solid support comprises material, which is selected from the group comprising organic and inorganic polymers.

Solid phases which can be solid or porous materials are also particularly suitable as matrices for binding or immobilizing nucleic acids. Such matrices are described for example in ‘Affinity Chromatography—a practical approach’ (P. D. G. Dean, W. S. Johnson, F. A. Middle (Ed.), IRL Press, Oxford, 1985): agarose, porous, particulate clay (aluminium oxide), cellulose, dextran (high molecular glucose polymer), Eupergit™ (by Röhm Pharma, oxirane-derivatized acrylic beads; copolymer of methacrylamide, methylene-bis-acrylamide, glycidyl-methacrylate and/or allyl-glycidyl ether), glass, controlled pore glass (abbr. CPG) whereby glass surface is usually derivatized with silane-containing compounds, hydroxyalkyl methacrylate, polyacrylamide, Sephadex™ (a dextran-based gel e.g. by Amersham Pharmacia Biotech), Sepharose, Superose (cross-linked agaroses e.g. by Amersham Pharmacia Biotech) whereby Sepharose is obtainable with various linkers/spacers as well as with a variety of functional groups e.g. NHS esters, CNBr-activated, amino, carboxy, activate thiol, epoxy etc. (see Pharmacia LKB biotechnology, Affinity Chromatography—Principles and Methods, Sweden 1993), Sephacryl (Spherical allyl-dextran and N,N-methylene bisacrylamide), Superdex (spherical, consisting of cross-linked agarose and dextran e.g. by Amersham Pharmacia Biotech), trisacryl, paramagnetic particles, Toyopearl™ (TosoHaas., semirigid, macroporous, spherical matrix), nylon-based matrices, tentagel (by Rapp polymers), copolymers consisting of a low cross-linked polystyrene matrix which is modified with polyethylene glycol or polyoxyethylene whereby the polyethylene glycol or polyoxyethylene units carry various functional groups), polystyrene.

Other matrices are for example silica gel, alumosilicates, bentonite, porous ceramics, various metal oxides, hydroxyapatite, fibroin (natural silk), alginates, carrageen, collagen and polyvinyl alcohol.

In additional functionalized or derivatized membranes or surfaces can also be used as the matrix.

Matrices are derivatized using suitable functional groups to obtain matrices that are either already pre-activated or matrices which have to be activated by adding suitable agents. Examples of non-activated functional groups that can be used to derivatize matrices are amino, thiol, carboxyl, phosphate, hydroxy groups etc. Examples of activating derivatizations of matrices are function groups such as hydrazide, axide, aldehyde, bromoacetyl, 1,1′-carbonyldiimidazole, cyanogen bromide, epichloro-hydrin, epoxide (oxirane), N-hydroxysuccinimide and all other possible active esters, periodate, pyridyl disulfide and other mixed disulfides, tosyl chloride, tresyl chloride, vinyl sulfonyl, benzyl halogenides, isocyanates, photoreactive groups etc.

All matrices through which plasma and preferably also whole blood can be passed are particularly suitable for apheresis such as organic polymers based on for example methacrylates, natural polymers based for example on cross-linked sugar structures or also inorganic polymers based for example on glass structures (CPG, controlled pore glass). The solid phase modified with the ligands, i.e. nucleic acids and preferably functional nucleic acids, which is suitable for plasmapheresis or apheresis is filled into a housing made of glass, plastic or metal to form an apheresis device.

The immobilization may preferable by chemical immobilization, affinity immobilization, or magnetic immobilization.

A particularly preferred form of immobilization is chemical immobilization based on the following interactions whereby one of the elements providing such interaction is the hepcidin binding nucleic acid whereas the other element providing such interaction is immobilised on the support. Examples, the putting into practice of which is known by a person skilled in the art, include but are not limited to:
An amine and an activated carboxylic acid,
An amine plus an activated carbamate,
An amine and an isocyanate/isothiocyanate,
An amine plus a halide,
An amine plus a maleimide moiety,
An amine plus an aldehyde/ketone
A hydroxylamine or a hydrazide plus a ketone/an aldehyde,
An hydrazine derivative and an activated carboxylic acid,
An hydrazine and an isocyanate/isothiocyanate,s
An hydrazine plus a halide,
An hydrazine plus a maleimide moiety,
An hydrazine+an aldehyde/a ketone;
An hydrazine+an aldehyde/a ketone followed by reductive amination
An thiol plus a halide,
An thiol plus a maleimide,
An thiol plus an activated thiol,
An thiol plus a vinyl sulfone and other Michael addition reactions
An azide plus an alkyne plus Cu salts and other “click chemistry” interaction partners (Kolb et. Al. 2001),
An azide plus an activated carboxylic acid via Staudinger reaction utilising alkyl or aryl P(III) moieties,
An azide plus a trivalent phosphine attached to an electrophilic trap (Staudinger ligation),
An azide plus a phosphinothiol ester—traceless Staudinger ligation,
An azide plus an aldehyde/a ketone+PPh3 (Staudinger) to form an imine that can then be with optional reducuction to the corresponding amine,
An amine plus a carboxyl group—
An carboxylic acid functional group plus amino functionality such as amine, hydrazine,
A Cis-diol (e.g. as found on the 3′ terminus of RNA molecules) oxidised to di-aldehyde that then forms cyclic amines for example, with either amines or hydrazine derivatives after e.g. borohydride mediated reduction,
A thioester plus a cysteine—native ligation and derivatives,
A phosphotioate+an alpha-halocarbonyl containing conjugants,
A phosphate+an amine to phosphoramidate e.g. via phosphate activation
A phosphate+an alcohol to phosphodiester e.g. via activation,
An aldehyde to form secondary amines (after reduction with Borohydride), hydrazino groups to form hydrazones, semicarbazides to form semi-carbazones.
A Cysteine derivate+a thioester peptide
An epoxide plus amine
An alkene/an alkyne+a diene/diyne for Diels Alder reaction, and other Pericyclic reactions
Oxime formation through reacting aldehyde with a hydroxylamine
A hydroxy or amino+an epoxide

The above reactions or at least some thereof are, among others described by Hermanson (Hermanson, 2008).

A particularly preferred form of immobilization is affinity immobilization based on the following interactions whereby one of the elements providing such interaction is the hepcidin binding nucleic acid, preferably comprising a modification, whereas the other element providing such interaction is a ligand immobilised on the support, whereby the ligand binds the hepcidin binding nucleic acid or to the modification linked thereto: biotin-avidin interaction, biotin-neutravidin interaction, biotin-streptavidin interaction, interaction of antibody and antigen or hapten, interaction of two oligonucleotides, whereby the nucleic acid molecules consist of DNA, RNA, LNA, PNA or combinations thereof, interaction of calmodulin and calmodulin binding peptide, interaction of albumin and Cibracon Blue, interaction of a metal-chelator agent and metal-chelating support.

The nucleic acids according to the present invention and/or the antagonists according to the present invention may be used for the generation or manufacture of a medicament, pharmaceutical composition or a medical device. Furthermore, the nucleic acid according to the present invention which is also the nucleic acid molecule of the present invention can be used in each and any method disclosed herein, and in particular in any method for reducing the level of hepcidin in a body fluid of or from a subject, in any method for removing hepcidin form a body fluid of a subject and/or any method for the treatment of an anaemic patient, each in particular as disclosed herein.

Such medicament or a pharmaceutical composition according to the present invention contains at least one of the inventive nucleic acids, optionally together with at least one further pharmaceutically active compound, whereby the inventive nucleic acid preferably acts as pharmaceutically active compound itself. Such medicament or pharmaceutical composition comprises in a preferred embodiment at least a pharmaceutically acceptable carrier. Such carrier may be, e.g., water, buffer, PBS, glucose solution, preferably a 5% glucose salt balanced solution, starch, sugar gelatine or any other acceptable carrier substance. Such carriers are generally known to the one skilled in the art. It will be acknowledged by the person skilled in the art that any embodiments, use and aspects of or related to the medicament of the present invention is also applicable to the pharmaceutical composition of the present invention and vice versa.

The medical devices wherein the hepcidin binding nucleic acids according to the present invention can be used are selected from the group of medical devices for dialysis, medical devices for hemodialysis, medical devices for hemofiltration, medical devices for hemodiafiltration, medical devices for aphersis, and adsorber, but not limited to.

The individual components of an apheresis device are known to a person skilled in the art. Examples of commercial apheresis systems are the liposorber system from the Kaneka Corporation, the DALI system (direct adsorption of lipids) containing the haemoadsorption instrument 4008 ADS from Fresenius AG, Bad Homburg, the H.E.L.Psystem (heparin-induced extracorporeal LDL precipitation) from B. Braun AG, Melsungen the systems Ig-Therasorb, Rheosorb from PlasmaSelect AG, Teterow. The various components of a dialysis device are known to a person of skill in the art. Suitable dialysate or dialysis fluids are known to the skilled. person. Suitable membranes with appropriate pore size applicable for carrying out the present invention are also known in the art. The indication, diseases and disorders for the treatment and/or prevention of which the nucleic acids, the pharmaceutical compositions, medicaments and medical devices each in accordance with or prepared in accordance with the present invention are used or are intended to be used, result from the involvement, either direct or indirect, of hepcidin in the respective pathogenetic mechanism.

As mentioned in the introductory part, hepcidin is the key signal regulating iron homeostasis whereas high levels of human hepcidin result in reduced serum iron levels and low levels result in increased serum iron levels as shown in hepcidin-deficiency and hepcidin overexpressing mouse models (Nicolas, 2001; Nicolas, 2002a; Nicolas, 2002b; Nicolas, 2003).

As also mentioned herein, binding of hepcidin to ferroportin results in immediate internalisation of ferroportin and a subsequent and long lasting decrease of serum iron (Rivera, 2005), whereby the decrease of serum iron is a cause of anemia. Anemia is defined as an absolute reduction in the quantity of haemoglobin in the circulating blood and is often a symptom of a disease manifested by low haemoglobin and not an isolated diagnosis in itself. Anemia results from a medical condition that negatively impairs production and/or lifespan of red blood cells. Additionally, anemia can be a result of blood loss.

Therefore and to understand the development of anemia, based on the underlying mechanism anemia is grouped into three eitologic categories.
a) decreased red blood cell production,
b) increased red blood cell destruction, and
c) blood loss.

However, the three categories—decreased red blood cell production, increased red blood cell destruction, and blood loss—are not strictly separated from each other but can occur concomitantly or independantly from each other.

In many diseases a combination of said mechanisms can lead to anemia. Thus, neutralisation of hepcidin might be beneficial in many conditions of anemia.

As the hepcidin binding nucleic acids according to the present invention interact with or bind to human hepcidin, a skilled person will understand that the hepcidin binding nucleic acids according to the present invention can be used for the treatment, prevention and/or diagnosis of any disease of humans and animals as described herein. In connection therewith, it is to be acknowledged that the nucleic acid molecules according to the present invention can be used for the treatment and prevention of any of the diseases, disorders or conditions described herein.

In the following, and without wishing to be bound by any theory, the rationale for the use of the nucleic acid molecules according to the present invention in connection with the various diseases, disorders and conditions is provided, thus rendering the claimed therapeutic, preventive and diagnostic applicability of the nucleic acid molecules according to the present invention plausible. In order to avoid any unnecessary repetition, it should be acknowledged that due to the involvement of the hepcidin—ferroportin interaction as known to a person skilled in the art and as also outlined herein said interaction may be addressed by the nucleic acid molecules according to the present invention such that the claimed therapeutic and/or preventative effect is achieved.

Accordingly, diseases and/or disorders and/or diseased conditions for the treatment and/or prevention of which the medicament according to the present invention may be used include, but are not limited to anemia, hypoferremia, pica, conditions with elevated hepcidin level.

Preferably anemia is selected from the group of sideroblastic anemia, hypochromic microcytic anemia, anemia caused by chronic disease and/or disorder, anemia caused by inflammation, anemia caused by genetic disorders, anemia caused by acute infections and/or anemia caused by mutation in genes of iron metabolism and/or homeostatis.

The various chronic diseases and/or disorders that can cause anemia are selected from the group of chronic inflammation, cancer, autoimmune disease and/or autoimmune disorder, chronic infection, arteriosclerosis, atherosclerosis, and cirrhosis of the liver. Insofar, anemia which may be treated by a nucleic acid of the present invention, is an anemia which is caused by or associated with any one of said various chronic diseases and/or disorders. Moreover anemia can be one which is caused by cancer treatment, preferably chemotherapy.

Subgroups of chronic inflammation are chronic kidney disease, chronic obstructive pulmonary disease, multiple sclerosis, osteoarthritis, diabetes, obesity, cerebrovascular disease, congestive heart disease, congestive heart failure, myocardial infarction, coronary artery disease, peripheral occlusive arterial disease, pancreatitis, vasculitis, whereby such chronic kidney disease comprises renal disease, chronic renal failure, chronic kidney failure and/or caused by kidney dialysis, or kidney transplantation.

Subgroups of cancer are hepatocellular carcinoma, lymphoma, multiple myeloma, head-and-neck cancer, breast cancer, colorectal cancer, nonmyeloid cancers, renal cell carcinoma, non-small-cell lung cancer, tumors and brain tumors.

Subgroups of autoimmune diseases and/or disorders are rheumatoid arthritis, irritable bowel syndrome, systemic lupus erythrematosus and Crohn's disease.

Subgroup of chronic infection are viral infections, viral illness, bacterial infections and fungal infections, whereby the viral infections comprise, but are not limited to, hepatitis and HIV infection and the bacterial infections comprise, but are not limited to, H. pylori infection.

Anemia caused by inflammation is normocytic to microcytic, characterised by a low reticulocyte production index, total iron binding capacity (TIBC) is low or normal. Hepcidin, acute phase proteins and other markers of inflammation (for example: C-reactive protein) are increased in the case of anemia caused by inflammation. Anemia caused by inflammation is also referred to as anemia by inflammation.

The various genetic disorders that can cause anemia are selected from the group of the Castleman disease, Schnitzler's syndrome, iron refractory iron deficiency anemia (matriptase-2) (TMPRSS6) mutation, atransferrinemia, congenital dyserythropoietic anemia and hemoglobinopathies.

The various acute infection that can cause anemia are selected from the group of viral infection, bacterial infection and fungal infection, whereby viral infection, bacterial infection and fungal infection individually or in combination with each other can lead to sepsis.

The term “conditions with elevated hepcidin level” refers to a condition in a mammal, preferably a human, wherein the level of hepcidin in the body is elevated compared to the normal level of hepcidin for such a mammal, such as an elevated hepcidin serum level compared to the normal hepcidin serum level for the mammal. The normal hepcidin serum level is approximately 54 ng/mL in case of a human being (see user manual, enzyme-linked immunoassay for hepcidin that is commercially by DRG Diagnostics, Marburg, Germany. Elevated serum hepcidin levels can, among others, be determined by enzyme-linked immunoassay (commercially available kit by DRG Diagnostics, Marburg, Germany).

Accordingly, the patients for which the medicament according to the present invention may preferably be used include, but are not limited to patients which are treated with erythropoietin and other red cell stimulating therapies and preferably show a hypo-responsiveness to erythropoietin, whereby more preferably the patients have a chronic kidney disease or suffering from cancer, whereby cancer is selected from the group of hepatocellular carcinoma, lymphoma, multiple myeloma, head-and-neck cancer, breast cancer, colorectal cancer, nonmyeloid cancers, renal cell carcinoma, non-small-cell lung cancer, tumors and brain tumors.

In a further embodiment, the medicament according to the invention comprises a further pharmaceutically active compound. Such further pharmaceutically active compound is preferably one that can modulate the activity, concentration or expression of hepcidin or ferroportin. Such compound is preferably a pro-hepcidin cleaving protease inhibitor, a pro-hepcidin antibody, a ferroportin-antagonist such as, e.g. a ferroportin-antibody, a JAK2 inhibitor, GDF15, a BMP modulator, a soluble haemojuvelin or TGF-beta inhibitor.

Other further pharmaceutically active compounds which may be used together with or contained in the medicament comprising a nucleic acid according to the invention are those that are known and/or used for treating anemia and/or inflammatory conditions, whereby the treatment of the inflammatory conditions positively influences anemia. Such pharmaceutically active compounds are selected from the group comprising iron supplements, vitamin supplements, red cell production stimulators, antibiotics, anti-inflammatory biologics, suppressors of the immune system, anti-thrombolytics, statins, vasopressors and inotropic compounds.

Non-limiting examples of iron supplements are ferrous sulphate, ferrous gluconate, iron dextran, sodium ferric gluconate, ferric carboxymaltose, iron-hydroxide polymaltose, iron fumarat, iron saccharose and iron-hydroxide sucrose.

Non-limiting examples of vitamin supplements are vitamin C, folic acid, vitamin B12, vitamin B6 and vitamin D.

Non-limiting examples of red cell production stimulators are erythropoietin, Epoetin, Darbepoetin, CERA, HIF prolyl-hydroxylase inhibitors (for example FG-2216 and FG-4592) and other erythropoiesis stimulating agents.

Non-limiting examples of antibiotics are aminoglycosides, beta-lactam antibiotics, peptide antibiotics, gryase inhibitors, lincosamide, macrolide antibiotics, nitroimidazole derivates, polypeptide antibiotics, sulfonamides, tetracycline and trimethoprim.

Non-limiting examples of anti-inflammatory biologics are
a) IL-6-receptor antagonists such as, e.g., Tocilizumab or Atlizumab,
b) TNF-antagonists such as, e.g., Etanercept, Infliximab, Adalimumab, Certolizumab,
c) IL-1 receptor antagonists such as, e.g., Anakinra, and
d) CD20 binding molecules such as, e.g., Rituximab and Ibiritumab.

Non-limiting examples of suppressors of the immune system are azathioprin, brequinar, calcineurin inhibitors, chlorambucil, cyclosporin A, deoxyspergualin, leflunomide, methotrexate, mizoribin, mycophenolate mofetil, rapamycin, tacroliums and thalidomide.

Non-limiting examples of anti-inflammatory agents are PDE4 inhibitors such as roflumilast and corticosteriods such as prednisolone, methylprednisolone, hydrocortisone, dexamethason, triamcinolone, betamethasone, effervescent, budesonide, ciclesonide and fluticasone.

Non-limiting of anti-thrombolytics are activated human protein C such as Drotrecogin alfa.

Non-limiting examples of statins are Atorvastatin, Cerivastatin, Fluvastatin, Lovastatin, Mevastatin, Pitavastatin, Pravastatin, Rosuvastatin and Simvastatin.

Non-limiting examples of vasopressors and/or inotropic compounds are noradrenalin, vasopressin and dobutamin.

In a further embodiment, the medicament according to the invention comprises a further pharmaceutically active compound which is preferably one that can bind iron and removes iron from tissue or from circulation of an mammalian body and a human body in particular. Such pharmaceutically active compound is preferably selected from the group of iron chelating compounds. Combination of such a compound with a nucleic acid molecule according to the present invention will further reduce the physiological hepcidin concentration and thereby reduce cellular iron load.

Non-limiting examples of iron chelating compounds are curcumin, deferoxamine, deferasirox and deferiprone.

Finally, the further pharmaceutically active agent may be a modulator of the iron metabolism and/or iron homoestatis. Alternatively, or additionally, such further pharmaceutically active agent is a further, preferably a second species of the nucleic acids according to the present invention. Alternatively, the medicament comprises at least one more nucleic acid which binds to a target molecule different from hepcidin or exhibits a function which is different from the one of the nucleic acids according to the present invention. Preferably such at least one more nucleic acid exhibits a function similar or identical to the one of the one or several of the further pharmaceutically active compound(s) disclosed herein.

It is within the present invention that the medicament comprising a nucleic acid according to the invention, also referred to herein as the medicament of the (present) invention, is alternatively or additionally used, in principle, for the prevention of any of the disease disclosed in connection with the use of the medicament for the treatment of said diseases. Respective markers therefore, i.e. for the respective diseases are known to the ones skilled in the art. Preferably, the respective marker is hepcidin.

In one embodiment of the medicament of the present invention, such medicament for use in combination with other treatments for any of the diseases disclosed herein, particularly those for which the medicament of the present invention is to be used.

“Combination therapy” or “co-therapy” as preferably used herein, includes the administration of a medicament of the invention and at least a second agent as part of a treatment regimen intended to provide a beneficial effect from the co-actin of these therapeutic agents, i. e. the medicament of the present invention and said second agent. Administration of these therapeutic agents as or in combination typically is carried out over a defined time period (usually minutes, hours, days or weeks depending upon the combination selected).

“Combination therapy” may, but generally is not, intended to encompass the administration of two or more of therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to a subject a single capsule having a fixed ratio of each therapeutic agent or in multiple, single capsules for each of the therapeutic agents.

Sequential or substantially simultaneous administration of a therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of a specific combination of therapeutically effective agents may be administered by injection while the or an other therapeutic agent of the combination may be administered topically.

Alternatively, for example, all therapeutic agents may be administered topcially or all therapeutic agents may be administered by injection. The sequence in which the therapeutic agents are administered is not critical unless noted otherwise. Where the combination therapy further comprises a non-drug treatment, the non-drug treatment may be conducted at any suitable time as long as a beneficial effect from the combination of the therapeutic agents and the non-drug treatment is achieved. For example, in appropriate cases, the beneficial effect may still be achieved when the non-drug treatment is temporally stayed, perhaps by days or even weeks whereas the therapeutic agents are still administered.

As outlined in general terms above, the medicament according to the present invention can be administered, in principle, in any form known to the ones skilled in the art. A preferred route of administration is systemic administration, more preferably by parenteral administration, preferably by injection. Alternatively, the medicament may be administered locally. Other routes of administration comprise intramuscular, intraperitoneal, subcutaneous, per orum, intranasal, intratracheal and pulmonary with preference given to the route of administration that is the least invasive while ensuring efficiancy.

Parenteral administration is generally used for subcutaneous, intramuscular or intravenous injections and infusions. Additionally, one approach for parenteral administration employs the implantation of a slow-release or sustained-release systems, which assures that a constant level of dosage is maintained and which are well known to the ordinary skill in the art.

Furthermore, preferred medicaments of the present invention can be administered by the intranasal route via topical use of suitable intranasal vehicles, inhalants, or via transdermal routes, using those forms of transdermal skin patches well known to those of ordinary skill in that art. To be administered in the form of a transdermal delivery system, the dosage administration will typically be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

The medicament of the present invention will generally comprise an amount of the active component(s) effective for the therapy, including, but not limited to, a nucleic acid molecule of the present invention, preferably dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the medicament of the present invention.

In a further aspect the present invention is related to a pharmaceutical composition. Such pharmaceutical composition comprises at least one of the nucleic acids according to the present invention and preferably a pharmaceutically acceptable vehicle. Such vehicle can be any vehicle or any binder used and/or known in the art. More particularly such binder or vehicle is any binder or vehicle as discussed in connection with the manufacture of the medicament disclosed herein. In a further embodiment, the pharmaceutical composition comprises a further pharmaceutically active agent.

The preparation of a medicament and a pharmaceutical composition, respectively, is known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via a microdevice, microparticles or a sponge.

The pharmaceutical composition or medicament according to the invention may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form an injectable solution or suspension. Additionally, solid forms suitable for dissolving in liquid prior to injection can be formulated.

The medicaments and nucleic acid molecules, respectively, of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to form a lipid layer encapsulating the drug, which is well known to the ordinary person skilled in the art. For example, the nucleic acid molecules according to the invention can be provided as a complex with a lipophilic compound or non-immunogenic, high molecule weight compound constructed using methods known in the art. Additionally, liposomes may bear such nucleic acid molecules on their surface for targeting and carrying cytotoxic agents internally to mediate cell killing. An example of nucleic-acid associated complexes is provided in U.S. Pat. No. 6,011,020.

The medicaments and nucleic acid molecules, respectively, of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues Furthermore, the medicaments and nucleic acid molecules, respectively, of the present invention may be coupled to a class of biodegradable polymers useful in achieving controlled release of a drug, for example, polylactic acid, polyepsilon capro lactone, polyhydroxy butyric acid, polyorthoesters, polyacetals, polydihydropyrans, polycyanoacrylates and cross-linked or amphipathic block copolymers of hydrogels.

If desired, the pharmaceutical composition and medicament, respectively, to be administered may also contain amounts, typically minor amounts, of non-toxic auxiliary substances such as wetting or emulsifying agents, pH buffering agents, and other substances such as for example, sodium acetate, and triethanolamine oleate.

The dosage regimen utilizing the nucleic acid molecules and medicaments, respectively, of the present invention is selected in accordance with a variety of factors including type, species, age, weight, sex and medical condition of the patient; the severity of the condition to be treated; the route of administration; the renal and hepatic function of the patient; and the particular nucleic acid according to the invention or salt thereof employed. An ordinarily skilled physician or veterinarian can readily determine and prescribe the effective amount of the drug required to prevent, counter or arrest the progress of the condition.

Effective plasma levels of the nucleic acid according to the present invention preferably range from 500 fM to 500 μM in the treatment of any of the diseases disclosed herein.

The nucleic acid molecules and medicaments, respectively, of the present invention may preferably be administered in a single daily dose, every second or third day, weekly, every second week, in a single monthly dose or every third month.

It is within the present invention that the medicament as described herein constitutes the pharmaceutical composition disclosed herein.

In a further aspect the present invention is related to a method for the treatment of a subject who is in need of such treatment, whereby the method comprises the administration of a pharmaceutically effective amount of at least one of the nucleic acids according to the present invention. In an embodiment, the subject suffers from a disease or is at risk to develop such disease, whereby the disease is any one of those disclosed herein, particularly any one of those diseases disclosed in connection with the use of any of the nucleic acids according to the present invention for the manufacture of a medicament.

It is to be understood that the nucleic acid as well as the antagonists according to the present invention can be used not only as a medicament or for the manufacture of a medicament, but also for cosmetic purposes, particularly with regard to the involvement of hepcidin in inflamed regional skin lesions. Therefore, a further condition or disease for the treatment or prevention of which the nucleic acid, the medicament and/or the pharmaceutical composition according to the present invention can be used, is inflamed regional skin lesions.

As preferably used herein, the term treatment comprises in a preferred embodiment additionally or alternatively prevention and/or follow-up.

As preferably used herein, the terms disease and disorder shall be used in an interchangeable manner, if not indicated to the contrary.

As used herein, the term comprise is preferably not intended to limit the subject matter followed or described by such term. However, in an alternative embodiment the term comprises shall be understood in the meaning of containing and thus as limiting the subject matter followed or described by such term.

The nucleic acid according to the present invention can be detected and quantified by a process using a capture probe and detection probe as described in WO/2008/052774 which is incorporated herein by reference.

The various SEQ.ID. Nos., the chemical nature of the nucleic acid molecules according to the present invention and the target molecules hepcidin as used herein, the actual sequence thereof and the internal reference number is summarized in the following table.

| | RNA/ | | |
|---|---|---|---|
| Seq.-ID | Peptide | Sequence | Internal Reference |
| 1 | L-peptide | DTHFPICIFCCGCCHRSKCGMCCKT | Human hepcidin, human hepcidin-25 |
| 2 | L-peptide | DTHFPICIFCCGCCHRSKCGMCCKT | Hepcidin-25 of Macaca mulatta |
| | | | (rhesus monkey) |
| 3 | L-peptide | DTHFPICIFCCGCCHRSKCGMCCKT | Hepcidin-25 of Macaca fascularis |
| | | | (cynomolgus monkey) |
| 4 | L-peptide | DTHFPICIFCCGCCRKAICGMCCKT | Hepcidin-25 of Sus scrofa (pig) |
| 5 | L-peptide | DTNFPICIFCCKCCNNSQCGICCKT | Hepcdin-25 of Mus musculus (mouse) |
| 6 | L-peptide | DTNFPICLFCCKCCKNSSCGLCCIT | Hepcidin-25 of Rattus norvegicus |
| | | | (rat) |
| 7 | D-peptide | DTHFPICIFCCGCCHRSKCGMCCKT-Biotin | Biotinylated human D-hepcidin-25 |
| 8 | L-peptide | ICIFCCGCCHRSKCGMCCKT | human hepcidin-20 |
| 9 | L-peptide | FPICIFCCGCCHRSKCGMCCKT | human hepcidin-22 |
| 10 | D-RNA | GCACUCGUAAAGUAGAGGGACCCAGUCC | 223-C5-001 |
| | | GGCGUGAUAGUGCCGAGUGC | |
| 11 | D-RNA | GCACUUGUAAAGUAGAGGGACCCAGUCC | 223-B5-001 |
| | | GGCGUGAUAGUGCCGAGUGC | |
| 12 | D-RNA | GCAUUCGUAAAGUAGAGGGACCCAGUCCGGCGUGAUA | 223-A5-001 |
| | | GUGCCGAGUGC | |
| 13 | D-RNA | GCACUCGUAAAGUAGAGGGACCUAGUCCGGCGUGAUA | 223-A3-001 |
| | | GUGCCGGGUGC | |
| 14 | D-RNA | GCACUCGUAAAGUAGAGGGACCUAGUCCGGCGUGAUA | 223-F5-001 |
| | | GUGCCGAGUGC | |
| 15 | D-RNA | GCACUCGUAAAGUAGAGGGACUCAGUCCGGCGUGAUA | 223-G4-001 |
| | | GUGCCGAGUGC | |
| 16 | D-RNA | GCACUCGUAAAGUAGAGGGAUACAGUCCGGCGUGAUA | 223-A4-001 |
| | | GUGACGAGUGC | |
| 17 | D-RNA | CGUGUGUAAAGUAGAGGCAGGUAAUCUGCGGAGUGUU | 229-C2-001 |
| | | AGUUCCACACG | |
| 18 | D-RNA | CGCGUGUAAAGUAGAGGCAGGUAAUCUGCGGAGUGUU | 229-B4-001 |
| | | AGUUCCACACG | |
| 19 | D-RNA | CGUGUGUAAAGUAGAGGCAGGCAAUCUGCGGAGUGUU | 229-E2-001 |
| | | AGUUCCACACG | |
| 20 | D-RNA | CGUGUGUAAAGUAGAGGACAAUUGUCGGCGUGAUAGU | 229-B1-001 |
| | | GCCACACG | |
| 21 | D-RNA | GCUGUGUAAAGUAGAGGACAAUUGUCGGCGUGAUAGU | 229-B1-002 |
| | | GCCACAGC | |
| 22 | D-RNA | CGUGUGUAAAGUAGAGGACAAUAGUCGGCGUGAGAGU | 229-G1-001 |
| | | GCCACACG | |
| 23 | D-RNA | CGUGAAAAGUAGAAACUUGUCGAAAGCAAGUAGCGUG | 229-C4-001 |
| | | AUAGUGCCACG | |
| 24 | D-RNA | CGUGCUGGCGUGAUAGUGCUCCAGGUUCUGGAUAAAG | 229-D1-001 |
| | | UAGAGAGCACG | |
| 25 | D-RNA | CGUGCGAAGGAGUGAUAAGUGUUUCUGACUUUCUUCC | 229-E1-001 |
| | | AGACUCCCACG | |
| 26 | D-RNA | CACUCGUAAAGUAGAGGGACCCAGUCCGGCGUGAUAGU | 223-C5-002 |
| | | GCCGAGUG | |
| 27 | D-RNA | CGCGCGUAAAGUAGAGGGACCCAGUCCGGCGUGAUAGU | 223-C5-006 |
| | | GCCGCGCG | |
| 28 | D-RNA | GCGCGUAAAGUAGAGGGACCCAGUCCGGCGUGAUAGU | 223-C5-007 |
| | | GCCGCGC | |
| 29 | L-RNA | GCACUCGUAAAGUAGAGGGACCCAGUCCGGCGUGAUAG | 223-C5-001 |
| | | UGCCGAGUGC | |
| 30 | L-RNA | GCACUUGUAAAGUAGAGGGACCCAGUCCGGCGUGAUA | 223-B5-001 |
| | | GUGCCGAGUGC | |
| 31 | L-RNA | GCAUUCGUAAAGUAGAGGGACCCAGUCCGGCGUGAUA | 223-A5-001 |
| | | GUGCCGAGUGC | |
| 32 | L-RNA | GCACUCGUAAAGUAGAGGGACCUAGUCCGGCGUGAUA | 223-A3-001 |
| | | GUGCCGGGUGC | |
| 33 | L-RNA | GCACUCGUAAAGUAGAGGGACCUAGUCCGGCGUGAUA | 223-F5-001 |
| | | GUGCCGAGUGC | |
| 34 | L-RNA | GCACUCGUAAAGUAGAGGGACUCAGUCCGGCGUGAUA | 223-G4-001 |
| | | GUGCCGAGUGC | |
| 35 | L-RNA | GCACUCGUAAAGUAGAGGGAUACAGUCCGGCGUGAUA | 223-A4-001 |
| | | GUGACGAGUGC | |
| 36 | L-RNA | CGUGUGUAAAGUAGAGGCAGGUAAUCUGCGGAGUGUU | 229-C2-001 |
| | | AGUUCCACACG | |
| 37 | L-RNA | CGCGUGUAAAGUAGAGGCAGGUAAUCUGCGGAGUGUU | 229-B4-001 |
| | | AGUUCCACACG | |
| 38 | L-RNA | CGUGUGUAAAGUAGAGGCAGGCAAUCUGCGGAGUGUU | 229-E2-001 |
| | | AGUUCCACACG | |
| 39 | L-RNA | CGUGUGUAAAGUAGAGGACAAUUGUCGGCGUGAUAGU | 229-B1-001 |
| | | GCCACACG | |
| 40 | L-RNA | GCUGUGUAAAGUAGAGGACAAUUGUCGGCGUGAUAGU | 229-B1-002 |
| | | GCCACAGC | |
| 41 | L-RNA | CGUGUGUAAAGUAGAGGACAAUAGUCGGCGUGAGAGU | 229-G1-001 |
| | | GCCACACG | |
| 42 | L-RNA | CGUGAAAAGUAGAAACUUGUCGAAAGCAAGUAGCGUG | 229-C4-001 |
| | | AUAGUGCCACG | |
| 43 | L-RNA | CGUGCUGGCGUGAUAGUGCUCCAGGUUCUGGAUAAAG | 229-D1-001 |
| | | UAGAGAGCACG | |
| 44 | L-RNA | CGUGCGAAGGAGUGAUAAGUGUUUCUGACUUUCUUCC | 229-E1-001 |
| | | AGACUCCCACG | |
| 45 | L-RNA | CACUCGUAAAGUAGAGGGACCCAGUCCGGCGUGAUAGU | 223-C5-002 |
| | | GCCGAGUG | |
| 46 | L-RNA | CGCGCGUAAAGUAGAGGGACCCAGUCCGGCGUGAUAGU | 223-C5-006 |
| | | GCCGCGCG | |
| 47 | L-RNA | GCGCGUAAAGUAGAGGGACCCAGUCCGGCGUGAUAGUGCCGCGC | 223-C5-007 |
| 48 | L-RNA | 5′-40-kDa-PEG- | 223-C5-001-5′- |
| | | GCACUCGUAAAGUAGAGGGACCCAGUCCGGCGUGAUAGUGCCGAGUGC | PEG |
| 49 | D-RNA | AGGCGUAAAGUAGAGGGGCUGAGCCCGGCGUGUUAGUGCCGCCU | 238-A1-001 |
| 50 | D-RNA | AGGCGUAAAGUAGAGGGACGUAGUCCGGCGUGAUAGUGCCGCCU | 238-E2-001 |
| 51 | D-RNA | CGUGUGUAAAGUAGAGGCAGAUAAUCUGCGGAGUGUUAGUUCCACACG | 237-A7-001 |
| 52 | D-RNA | CGUGAAAAGUAGAAACUUGUCGAAAGCAAGCAGCGUGAUAGUGCCACG | 236-G2-001 |
| 53 | D-RNA | CGUGAAAAGUUGAAAUUUGUUGGAAUCAAGCAGGGAUAUAGUGCCACG | 236-D1-001 |
| 54 | D-RNA | AGCGUGUCGUAUGGGAUAAGUAAAUGAGGAGUUGGAGGAAGGGUGCGCU | 238-D2-001 |
| 55 | D-RNA | AGCGUGUCGUAUGGGAUUAAGUAAAUGAGGAGUUGGAGGAAGGGCAUGC | 238-D4-001 |
| | | U | |
| 56 | D-RNA | AGUGUGUCGUAUGGGAUAAGUAAAUGAGGGGUUGGAGG | 238-H1-001 |
| | | AAGGAUGCGCU | |
| 57 | D-RNA | AGUGUGUCAUAUGGGAUAAGUAAAUGAGGAGUUGGAGG | 238-A2-001 |
| | | AAAGGCAUGCU | |
| 58 | D-RNA | AGCGUGCCGGAUGGGAUAAGUAAAUGAGGAGUUGGAGG | 238-G2-001 |
| | | AAGGGUGCGCU | |
| 59 | D-NA | AGCGUGCCGUAUGGGAUAAGUAAAUGAGGAGUAGGAGG | 238-G4-001 |
| | | AAGGGUACGCU | |
| 60 | D-RNA | AGCGCGCCGUAUGGGAGAAGUAAAUGAGGAGUUGGAGG | 238-G3-001 |
| | | AAGGGCGCGCU | |
| 61 | D-RNA | AGGCUCGGACAGCCGGGGGACACCAUAUACAGACUACGA | 238-C4-001 |
| | | UACGGGCCU | |
| 62 | D-RNA | AGGCUCGGACGGCCGGGGGACACCAUAUACAGACUACUA | 238-E3-001 |
| | | UACGGGCCU | |
| 63 | D-RNA | AGGCCCGGACAGCCGGGGGACACCAUAUACAGACUACUA | 238-F2-001 |
| | | UACGGGCCU | |
| 64 | D-RNA | AGGCUUGGGCGGCCGGGGGACACCAUAUACAGACUACU | 238-A4-001 |
| | | AUACGAGCCU | |
| 65 | D-RNA | AGACUUGGGCAGCCGGGGGACACCAUAUACAGACUACG | 238-E1-001 |
| | | AUACGAGUCU | |
| 66 | D-RNA | CGGGCGCCAUAGACCGUUAUUAAGCACUGUAACUACCG | 237-A5-001 |
| | | AACCGCGCCCG | |
| 67 | D-RNA | CGGGCGCCAUAGACCGUUAACUACAUAACUACCGAACC | 237-C5-001 |
| | | GUGCCCG | |
| 68 | D-RNA | CGGGCGCUACCGAACCCACUAAAACCAGUGCAUAGACC | 236-F2-001 |
| | | GCGCCCG | |
| 69 | D-RNA | CGGGCGCUACCGAACCGUCACGAAGACCAUAGACCGCG | 236-G4-001 |
| | | CCG | |
| 70 | D-RNA | CGAGCGCAACCGAACCUCUACCCAGACAUAGACCGCGC | 236-E3-001 |
| | | CCG | |
| 71 | D-RNA | GCACUCGUAAAGUAGAGGGACCAGUCCGGCGUGAUAG | 223-C5-008 |
| | | UGCCGAGUGC | |
| 72 | D-RNA | GUGUGUAAAGUAGAGGACAAUUGUCGGCGUGAUAGUG | 229-B1-003 |
| | | CCACAC | |
| 73 | D-RNA | GCGUGUAAAGUAGAGGACAAUUGUCGGCGUGAUAGUG | 229-B1-004 |
| | | CCACGC | |
| 74 | D-RNA | GCGCGUAAAGUAGAGGACAAUUGUCGGCGUGAUAGUG | 229-B1-005 |
| | | CCGCGC | |
| 75 | D-RNA | CGUGUGUAAAGUAGAGGACAAUUGUCGGCGUGAUAGU | 229-B1-006 |
| | | GCCACAC | |
| 76 | D-RNA | GCCGUGUAAAGUAGAGGACAAUUGUCGGCGUGAUAGU | 229-B1-007 |
| | | GCCACGGC | |
| 77 | D-RNA | GCGGUGUAAAGUAGAGGACAAUUGUCGGCGUGAUAGU | 229-B1-008 |
| | | GCCACCGC | |
| 78 | D-RNA | GCUGCGUAAAGUAGAGGACAAUUGUCGGCGUGAUAGU | 229-B1-009 |
| | | GCCGCAGC | |
| 79 | D-RNA | GCUGGGUAAAGUAGAGGACAAUUGUCGGCGUGAUAGU | 229-B1-010 |
| | | GCCCCAGC | |
| 80 | D-RNA | GCGGCGUAAAGUAGAGGACAAUUGUCGGCGUGAUAGU | 229-B1-011 |
| | | GCCGCCGC | |
| 81 | D-RNA | GCGCGCGUAUGGGAUUAAGUAAAUGAGGAGUUGGAGG | 238-D4-002 |
| | | AAGGCGCGC | |
| 82 | D-RNA | GCGCGCGUAUGGGAUAAGUAAAUGAGGAGUUGGAGGA | 238-D4-003 |
| | | AGGCGCGC | |
| 83 | D-RNA | GGCGCGUAUGGGAUUAAGUAAAUGAGGAGUUGGAGGA | 238-D4-004 |
| | | AGGCGCC | |
| 84 | D-RNA | GGCGCGUAUGGGAUAAGUAAAUGAGGAGUUGGAGGAA | 238-D4-005 |
| | | GGCGCC | |
| 85 | D-RNA | GGUGUCGUAUGGGAUUAAGUAAAUGAGGAGUUGGAGG | 238-D4-006 |
| | | AAGGGCAUC | |
| 86 | D-RNA | GGUGUCGUAUGGGAUAAGUAAAUGAGGAGUUGGAGGA | 238-D4-007 |
| | | AGGGCAUC | |
| 87 | D-RNA | GCGCCGUAUGGGAUUAAGUAAAUGAGGAGUUGGAGGA | 238-D4-008 |
| | | AGGGCGC | |
| 88 | D-RNA | GCGCCGUAUGGGAUAAGUAAAUGAGGAGUUGGAGGAA | 238-D4-009 |
| | | GGGCGC | |
| 89 | D-RNA | GGCGCCGUAUGGGAUUAAGUAAAUGAGGAGUUGGAGG | 238-D4-010 |
| | | AAGGGCGCC | |
| 90 | D-RNA | GGCGCCGUAUGGGAUAAGUAAAUGAGGAGUUGGAGGA | 238-D4-011 |
| | | AGGGCGCC | |
| 91 | D-RNA | GGCGUCGUAUGGGAUUAAGUAAAUGAGGAGUUGGAGG | 238-D4-012 |
| | | AAGGGCGCC | |
| 92 | D-RNA | GGCGUCGUAUGGGAUAAGUAAAUGAGGAGUUGGAGGA | 238-D4-013 |
| | | AGGGCGCC | |
| 93 | D-RNA | GGCUCGGACAGCCGGGGGACACCAUAUACAGACUACGA | 238-C4-002 |
| | | UACGGGCC | |
| 94 | D-RNA | GCUCGGACAGCCGGGGGACACCAUAUACAGACUACGAU | 238-C4-003 |
| | | ACGGGC | |
| 95 | D-RNA | CUCGGACAGCCGGGGGACACCAUAUACAGACUACGAUA | 238-C4-004 |
| | | CGGG | |
| 96 | D-RNA | GCCCGGACAGCCGGGGGACACCAUAUACAGACUACGAU | 238-C4-005 |
| | | ACGGGC | |
| 97 | D-RNA | GGCCGGACAGCCGGGGGACACCAUAUACAGACUACGAU | 238-C4-006 |
| | | ACGGCC | |
| 98 | D-RNA | GCGGAGACAGCCGGGGGACACCAUAUACAGACUACGAU | 238-C4-007 |
| | | AUCCGU | |
| 99 | D-RNA | AGGCUGACAGCCGGGGGACACCAUAUACAGACUACGAU | 238-C4-008 |
| | | AGGCCU | |
| 100 | D-RAN | GGCCUGACAGCCGGGGGACACCAUAUACAGACUACGAU | 238-C4-009 |
| | | AAGGCU | |
| 101 | D-RNA | GCGCGGACAGCCGGGGGACACCAUAUACAGACUACGAU | 238-C4-010 |
| | | ACGCGC | |
| 102 | D-RNA | GCCGGACAGCCGGGGGACACCAUAUACAGACUACGAUA | 238-C4-011 |
| | | CGGC | |
| 103 | D-RNA | GGCGGACAGCCGGGGGACACCAUAUACAGACUACGAUA | 238-C4-012 |
| | | CGCC | |
| 104 | D-RNA | GGCCGACAGCCGGGGGACACCAUAUACAGACUACGAUA | 238-C4-013 |
| | | GGCC | |
| 105 | D-RNA | GCGCGACAGCCGGGGGACACCAUAUACAGACUACGAUA | 238-C4-014 |
| | | GCGC | |
| 106 | D-RNA | GGCCGGACAGCCGGAGGACACCAUAUACAGACUACGAU | 238-C4-024 |
| | | ACGGCC | |
| 107 | D-RNA | GGCCGGACAGCCGGCGGACACCAUAUACAGACUACGAUACGGCC | 238-C4-025 |
| 108 | D-RNA | GGCCGGACAGCCGGGAGGACACCAUAUACAGACUACGAUACGGCC | 238-C4-062 |
| 109 | L-RNA | 5′ UCCAGGUUCUGGA | |
| 110 | L-RNA | AGGCGUAAAGUAGAGGGGCUGAGCCCGGCGUGUUAGUGCCGCCU | 238-A1-001 |
| 111 | L-RNA | AGGCGUAAAGUAGAGGGACGUAGUCCGGCGUGAUAGUGCCGCCU | 238-E2-001 |
| 112 | L-RNA | CGUGUGUAAAGUAGAGGCAGAUAAUCUGCGGAGUGUUAGUUCC | 237-A7-001 |
| | | ACACG | |
| 113 | L-RNA | CGUGAAAAGUAGAAACUUGUCGAAAGCAAGCAGCGUGAUAGUG | 236-G2-001 |
| | | CCACG | |
| 114 | L-RNA | CGUGAAAAGUUGAAAUUUGUUGGAAUCAAGCAGGGAUAUAGUG | 236-D1-001 |
| | | CCACG | |
| 115 | L-RNA | AGCGUGUCGUAUGGGAUAAGUAAAUGAGGAGUUGGAGGAAGGG | 238-D2-001 |
| | | UGCGCU | |
| 116 | L-RNA | AGCGUGUCGUAUGGGAUUAAGUAAAUGAGGAGUUGGAGGAAGG | 238-D4-001 |
| | | GCAUGCU | |
| 117 | L-RNA | AGUGUGUCGUAUGGGAUAAGUAAAUGAGGGGUUGGAGGAAGGA | 238-H1-001 |
| | | UGCGCU | |
| 118 | L-RNA | AGUGUGUCAUAUGGGAUAAGUAAAUGAGGAGUUGGAGG | 238-A2-001 |
| | | AAAGGCAUGCU | |
| 119 | L-RNA | AGCGUGCCGGAUGGGAUAAGUAAAUGAGGAGUUGGAGGA | 238-G2-001 |
| | | AGGGUGCGCU | |
| 120 | L-RNA | AGCGUGCCGUAUGGGAUAAGUAAAUGAGGAGUAGGAGGA | 238-G4-001 |
| | | AGGGUACGCU | |
| 121 | L-RNA | AGCGCGCCGUAUGGGAGAAGUAAAUGAGGAGUUGGAGGA | 238-G3-001 |
| | | AGGGCGCGCU | |
| 122 | L-RNA | AGGCUCGGACAGCCGGGGGACACCAUAUACAGACUACGA | 238-C4-001 |
| | | UACGGGCCU | |
| 123 | L-RNA | AGGCUCGGACGGCCGGGGGACACCAUAUACAGACUACUA | 238-E3-001 |
| | | UACGGGCCU | |
| 124 | L-RNA | AGGCCCGGACAGCCGGGGGACACCAUAUACAGACUACUA | 238-F2-001 |
| | | UACGGGCCU | |
| 125 | L-RNA | AGGCUUGGGCGGCCGGGGGACACCAUAUACAGACUACUA | 238-A4-001 |
| | | UACGAGCCU | |
| 126 | L-RNA | AGACUUGGGCAGCCGGGGGACACCAUAUACAGACUACG | 238-E1-001 |
| | | AUACGAGUCU | |
| 127 | L-RNA | CGGGCGCCAUAGACCGUUAUUAAGCACUGUAACUACCG | 237-A5-001 |
| | | AACCGCGCCCG | |
| 128 | L-RNA | CGGGCGCCAUAGACCGUUAACUACAUAACUACCGAACC | 237-C5-001 |
| | | GUGCCCG | |
| 129 | L-RNA | CGGGCGCUACCGAACCCACUAAAACCAGUGCAUAGACC | 236-F2-001 |
| | | GCGCCCG | |
| 130 | L-RNA | CGGGCGCUACCGAACCGUCACGAAGACCAUAGACCGCG | 236-G4-001 |
| | | CCG | |
| 131 | L-RNA | CGAGCGCAACCGAACCUCUACCCAGACAUAGACCGCG | 236-E3-001 |
| | | CCG | |
| 132 | L-RNA | GCACUCGUAAAGUAGAGGGACCAGUCCGGCGUGAUAG | 223-C5-008 |
| | | UGCCGAGUGC | |
| 133 | L-RNA | GUGUGUAAAGUAGAGGACAAUUGUCGGCGUGAUAGUG | 229-B1-003 |
| | | CCACAC | |
| 134 | L-RNA | GCGUGUAAAGUAGAGGACAAUUGUCGGCGUGAUAGUG | 229-B1-004 |
| | | CCACGC | |
| 135 | L-RNA | GCGCGUAAAGUAGAGGACAAUUGUCGGCGUGAUAGUG | 229-B1-005 |
| | | CCGCGC | |
| 136 | L-RNA | CGUGUGUAAAGUAGAGGACAAUUGUCGGCGUGAUAGU | 229-B1-006 |
| | | GCCACAC | |
| 137 | L-RNA | GCCGUGUAAAGUAGAGGACAAUUGUCCGGCGUGAUAGU | 229-B1-007 |
| | | GCCACGGC | |
| 138 | L-RNA | GCGGUGUAAAGUAGAGGACAAUUGUCGGCGUGAUAGU | 229-B1-008 |
| | | GCCACCGC | |
| 139 | L-RNA | GCUGCGUAAAGUAGAGGACAAUUGUCGGCGUGAUAGU | 229-B1-009 |
| | | GCCGCAGC | |
| 140 | L-RNA | GCUGGGUAAAGUAGAGGACAAUUGUCGGCGUGAUAGU | 229-B1-010 |
| | | GCCCCAGC | |
| 141 | L-RNA | GCGGCGUAAAGUAGAGGACAAUUGUCGGCGUGAUAGU | 229-B1-011 |
| | | GCCGCCGC | |
| 142 | L-RNA | GCGCGCGUAUGGGAUUAAGUAAAUGAGGAGUUGGAGG | 238-D4-002 |
| | | AAGGCGCGC | |
| 143 | L-RNA | GCGCGCGUAUGGGAUAAGUAAAUGAGGAGUUGGAGGA | 238-D4-003 |
| | | AGGCGCGC | |
| 144 | L-RNA | GGCGCGUAUGGGAUUAAGUAAAUGAGGAGUUGGAGGA | 238-D4-004 |
| | | AGGCGCC | |
| 145 | L-RNA | GGCGCGUAUGGGAUAAGUAAAUGAGGAGUUGGAGGAA | 238-D4-005 |
| | | GGCGCC | |
| 146 | L-RNA | GGUGUCGUAUGGGAUUAAGUAAAUGAGGAGUUGGAGG | 238-D4-006 |
| | | AAGGGCAUC | |
| 147 | L-RNA | GGUGUCGUAUGGGAUAAGUAAAUGAGGAGUUGGAGGA | 238-D4-007 |
| | | AGGGCAUC | |
| 148 | L-RNA | GCGCCGUAUGGGAUUAAGUAAAUGAGGAGUUGGAGGA | 238-D4-008 |
| | | AGGGCGC | |
| 149 | L-RNA | GCGCCGUAUGGGAUAAGUAAAUGAGGAGUUGGAGGAA | 238-D4-009 |
| | | GGGCGC | |
| 150 | L-RNA | GGCGCCGUAUGGGAUUAAGUAAAUGAGGAGUUGGAGG | 238-D4-010 |
| | | AAGGGCGCC | |
| 151 | L-RNA | GGCGCCGUAUGGGAUAAGUAAAUGAGGAGUUGGAGGA | 238-D4-011 |
| | | AGGGCGCC | |
| 152 | L-RNA | GGCGUCGUAUGGGAUUAAGUAAAUGAGGAGUUGGAGG | 238-D4-012 |
| | | AAGGGCGCC | |
| 153 | L-RNA | GGCGUCGUAUGGGAUAAGUAAAUGAGGAGUUGGAGGA | 238-D4-013 |
| | | AGGGCGCC | |
| 154 | L-RNA | GGCUCGGACAGCCGGGGGACACCAUAUACAGACUACGAUACG | 238-C4-002 |
| | | GGCC | |
| 155 | L-RNA | GCUCGGACAGCCGGGGGACACCAUAUACAGACUACGAUACGG | 238-C4-003 |
| | | GC | |
| 156 | L-RNA | CUCGGACAGCCGGGGGACACCAUAUACAGACUACGAUACGGG | 238-C4-004 |
| 157 | L-RNA | 5′ GACAAUAGUC | |
| 158 | L-RNA | GCCCGGACAGCCGGGGGACACCAUAUACAGACUACGAUACGG | 238-C4-005 |
| | | GC | |
| 159 | L-RNA | GGCCGGACAGCCGGGGGACACCAUAUACAGACUACGAUACGG | 238-C4-006 |
| | | CC | |
| 160 | L-RNA | GCGGAGACAGCCGGGGGACACCAUAUACAGACUACGAUAUCC | 238-C4-007 |
| | | GU | |
| 161 | L-RNA | AGGCUGACAGCCGGGGGACACCAUAUACAGACUACGAUAGGC | 238-C4-008 |
| | | CU | |
| 162 | L-RNA | GGCCUGACAGCCGGGGGACACCAUAUACAGACUACGAUAAGG | 238-C4-009 |
| | | CU | |
| 163 | L-RNA | GCGCGGACAGCCGGGGGACACCAUAUACAGACUACGAUACGC | 238-C4-010 |
| | | GC | |
| 164 | L-RNA | GCCGGACAGCCGGGGGACACCAUAUACAGACUACGAUACGGC | 238-C4-011 |
| 165 | L-RNA | GGCGGACAGCCGGGGGACACCAUAUACAGACUACGAUACGCC | 238-C4-012 |
| 166 | L-RNA | GGCCGACAGCCGGGGGACACCAUAUACAGACUACGAUAGGCC | 238-C4-013 |
| 167 | L-RNA | GCGCGACAGCCGGGGGACACCAUAUACAGACUACGAUAGCGC | 238-C4-014 |
| 168 | L-RNA | GGCCGGACAGCCGGAGGACACCAUAUACAGACUACGAUACG | 238-C4-024 |
| | | GCC | |
| 169 | L-RNA | GGCCGGACAGCCGGCGGACACCAUAUACAGACUACGAUACG | 238-C4-025 |
| | | GCC | |
| 170 | L-RNA | GGCCGGACAGCCGGGAGGACACCAUAUACAGACUACGAUAC | 238-C4-062 |
| | | GGCC | |
| 171 | L-RNA | 5′-NH₂- | 229-B1-002-5′-Amino |
| | | GCUGUGUAAAGUAGAGGACAAUUGUCGGCGUGAUAG | |
| | | UGCCACAGC | |
| 172 | L-RNA | 5′-NH₂- | 223-C5-001-5′-Amino |
| | | GCACUCGUAAAGUAGAGGGACCCAGUCCGGCGUGAUAGUGC | |
| | | CGAGUGC | |
| 173 | L-RNA | 5′-PEG- | 229-B1-002-5′-PEG |
| | | GCUGUGUAAAGUAGAGGACAAUUGUCGGCGUGAUAGUGCC | |
| | | ACAGC | |
| 174 | L-RNA | 5′-PEG- | 238-CR-006-5′_PEG |
| | | GGCCGGACAGCCGGGGGACACCAUAUACAGACUACGA | |
| | | UACGGCC | |
| 175 | L-RNA | 5′-PEG- | 238-D4-002-5′-PEG |
| | | GCGCGCGUAUGGGAUUAAGUAAAUGAGGAGUUGGAGGAAG | |
| | | GCGCGC | |
| 176 | L-RNA | 5′-PEG- | 238-D4-008-5′-PEG, NOX- |
| | | GCGCCGUAUGGGAUUAAGUAAAUGAGGAGUUGGAGGAAGG | H94 |
| | | GCG | |
| 177 | L-RNA | 5′-CCAUACGGCGC-HEG-HEG-NH₂- | 5′CP-11_NOX-H94 |
| 178 | L-RNA | 5′-Biotin-HEG-HEG-GCGCCCUUCCUCC | 3′DP-13_NOX-H94 |
| 179 | L-RNA | 5′-NH₂- | 238-D4-002-5′-Amino |
| | | GCGCGCGUAUGGGAUUAAGUAAAUGAGGAGUUGGAGGAAG | |
| | | GCGCGC | |
| 180 | L-RNA | 5′-NH₂- | 238-D4-008-5′-Amino = |
| | | GCGCCGUAUGGGAUUAAGUAAAUGAGGAGUUGGAGG | NOX-H94-002 |
| | | AAGGGCGC | |
| 181 | L-RNA | 5′-NH₂- | 238-C4-006-5′-Amino |
| | | GGCCGGACAGCCGGGGGACACCAUAUACAGACUACGA | |
| | | UACGGCC | |
| 182 | L-RNA | 5′ RKAUGGGAKUAAGUAAAUGAGGRGUWGGAGGAAR | |
| 183 | L-RNA | 5′ RKAUGGGAKAAGUAAAUGAGGRGUWGGAGGAAR | |
| 184 | L-RNA | 5′ GUAUGGGAUUAAGUAAAUGAGGAGUUGGAGGAAG | |
| 185 | L-RNA | 5′ GRCRGCCGGVGGACACCAUAUACAGACUACKAUA | |
| 186 | L-RNA | 5′ GRCRGCCGGVAGGACACCAUAUACAGACUACKAUA | |
| 187 | L-RNA | 5′ GACAGCCGGGGGACACCAUAUACAGACUACGAUA | |
| 188 | L-RNA | 5′ WAAAGUWGAR | |
| 189 | L-RNA | 5′ RGMGUGWKAGUKS | |
| 190 | L-RNA | 5′ GGGCUGAGCCC | |
| 191 | L-RNA | 5′ GCAGAUAAUCUGC | |
| 192 | L-RNA | 5′ GGACCAGUCC | |
| 193 | L-RNA | 5′ GGACCCAGUCC | |
| 194 | L-RNA | 5′ GGACCUAGUCC | |
| 195 | L-RNA | 5′ GGACUCAGUCC | |
| 196 | L-RNA | 5′ GCAGGUAAUCUGC | |
| 197 | L-RNA | 5′ GCAGGCAAUCUGC | |
| 198 | L-RNA | 5′ GACAAUUGUC | |
| 199 | L-RNA | 5′ UAAAGUAGAG | |
| 200 | L-RNA | 5′ AAAAGUAGAA | |
| 201 | L-RNA | 5′ AAAAGUUGAA | |
| 202 | L-RNA | 5′ GGGAUAUAGUGC | |
| 203 | L-RNA | 5′ GGCGUGAUAGUGC | |
| 204 | L-RNA | 5′ GGAGUGUUAGUUC | |
| 205 | L-RNA | 5′ GGCGUGAGAGUGC | |
| 206 | L-RNA | 5′ AGCGUGAUAGUGC | |
| 207 | L-RNA | 5′ GGCGUGUUAGUGC | |
| 208 | L-RNA | 5′ GGACGYAGUCC | |
| 209 | L-RNA | 5′ GGAUACAGUCC | |
| 210 | L-RNA | 5′ GCAGGYAAUCUGC | |
| 211 | L-RNA | 5′ GACAAUWGUC | |
| 212 | L-RNA | 5′ ACUUGUCGAAAGCAAGY | |
| 213 | L-RN | 5′-NH2-HEG-HEG-HEG- | NOX-H94-3xHEG-amino |
| | | GCGCCGUAUGGGAUUAAGUAAAUGAGGAGUUGGAGGAAGG | |
| | | GCGC | |
| 214 | L-RNA | 5′ GGACGUAGUCC | |
| 215 | L-RNA | 5′ AUUUGUUGGAAUCAAGCA | |
| 216 | L-RNA | 5′ GRCRGCCGGGGGACACCAUAUACAGACUACKAUA | |

The present invention is further illustrated by the figures, examples and the sequence listing from which further features, embodiments and advantages may be taken, wherein

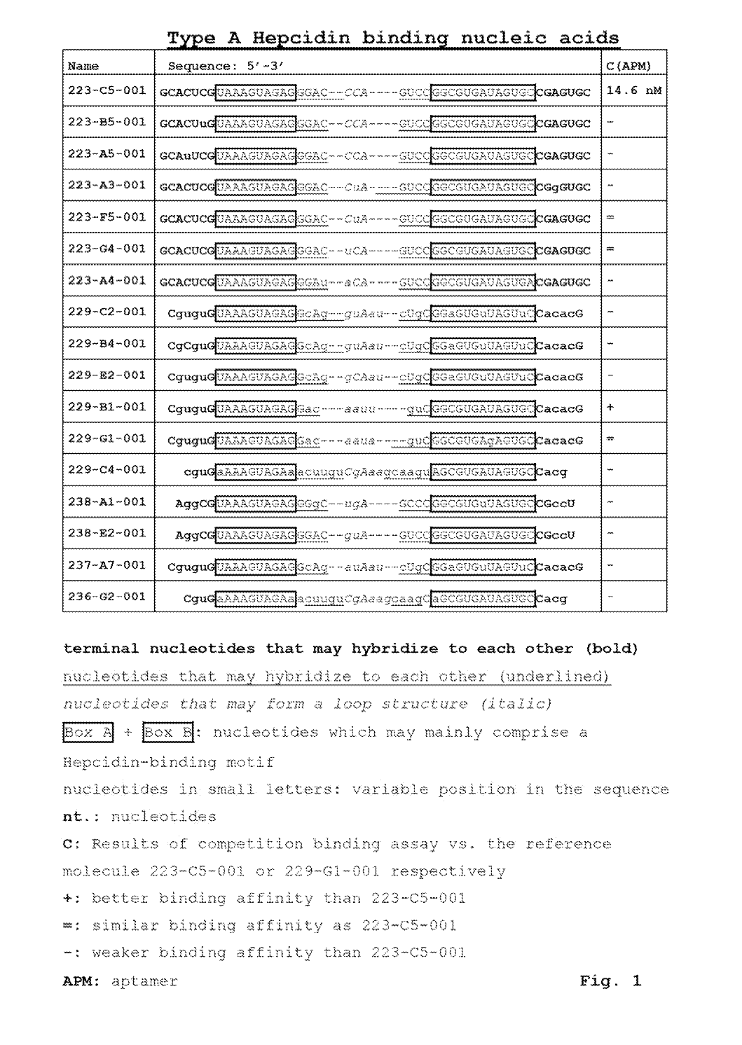

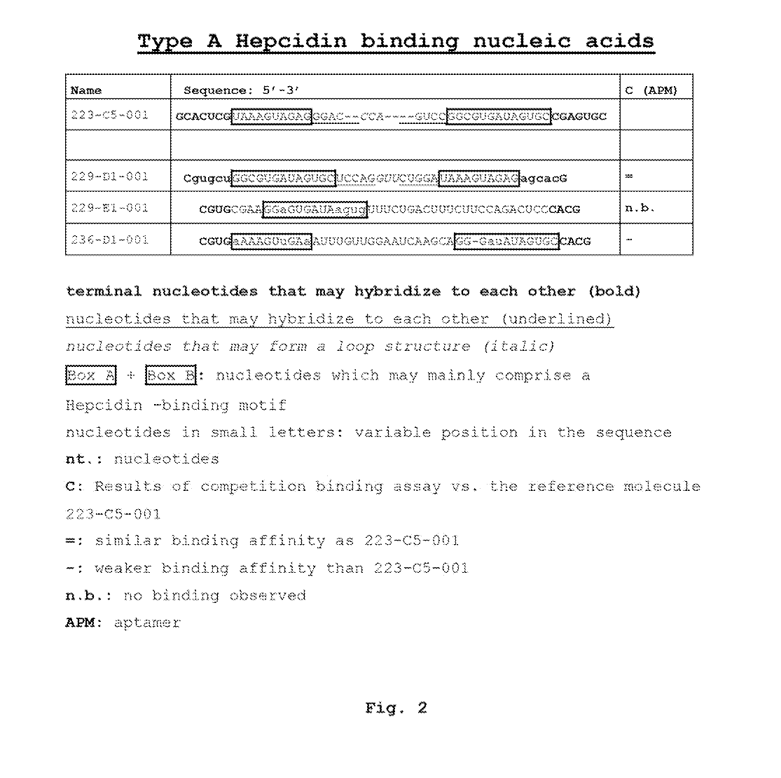

FIGS. 1 and 2 shows an alignment of sequences of Type A hepcidin binding nucleic acids;

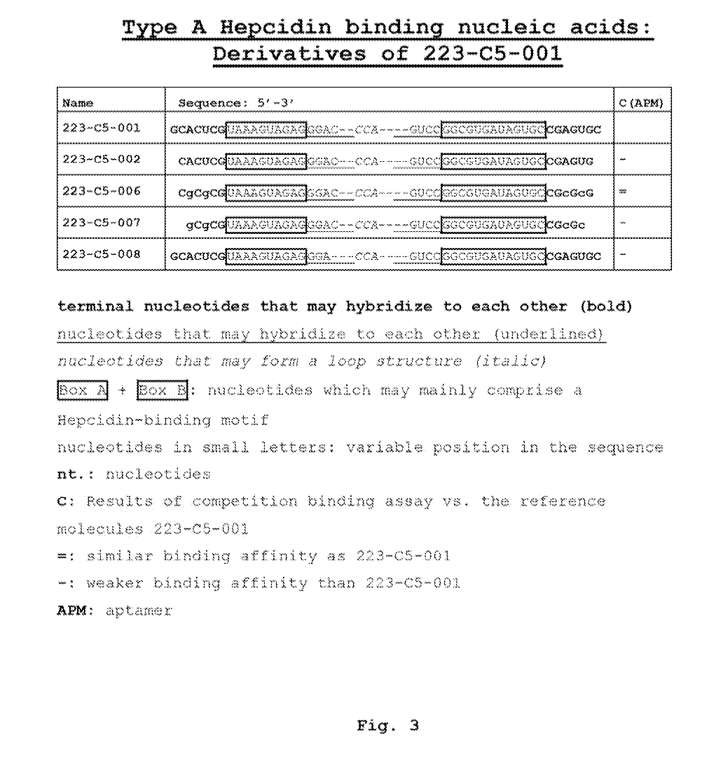

FIG. 3 shows derivatives of Type A hepcidin binding nucleic acid 223-C5-001;

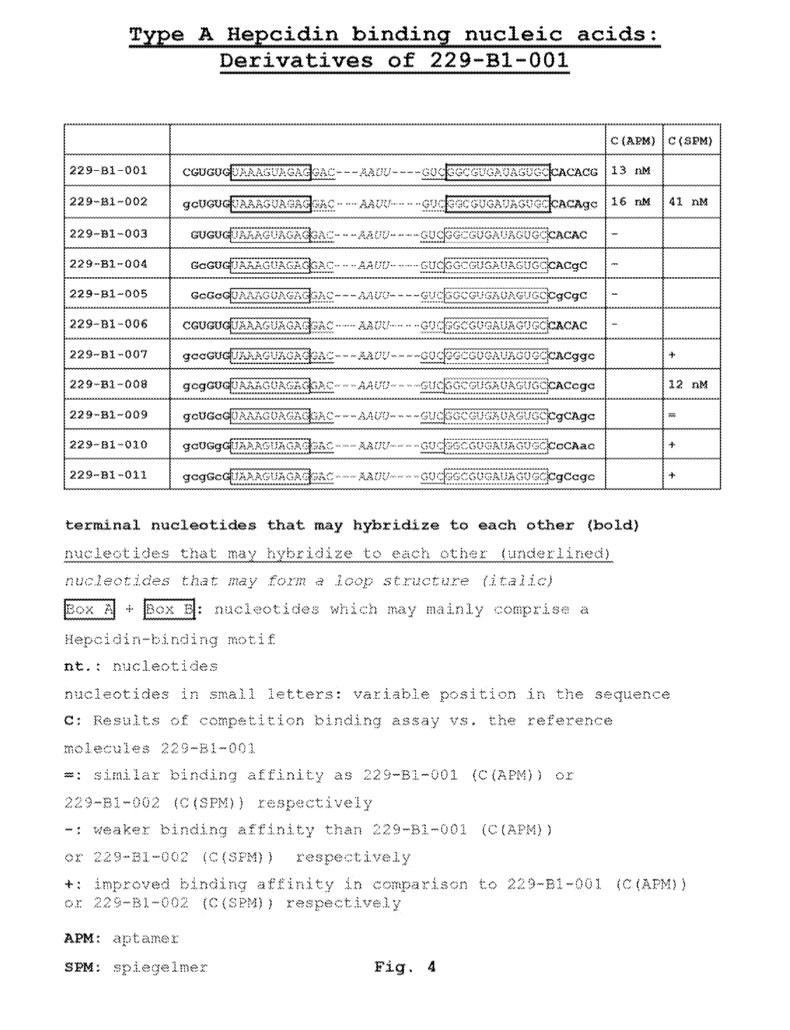

FIG. 4 shows derivatives of Type A hepcidin binding nucleic acid 229-B1-001;

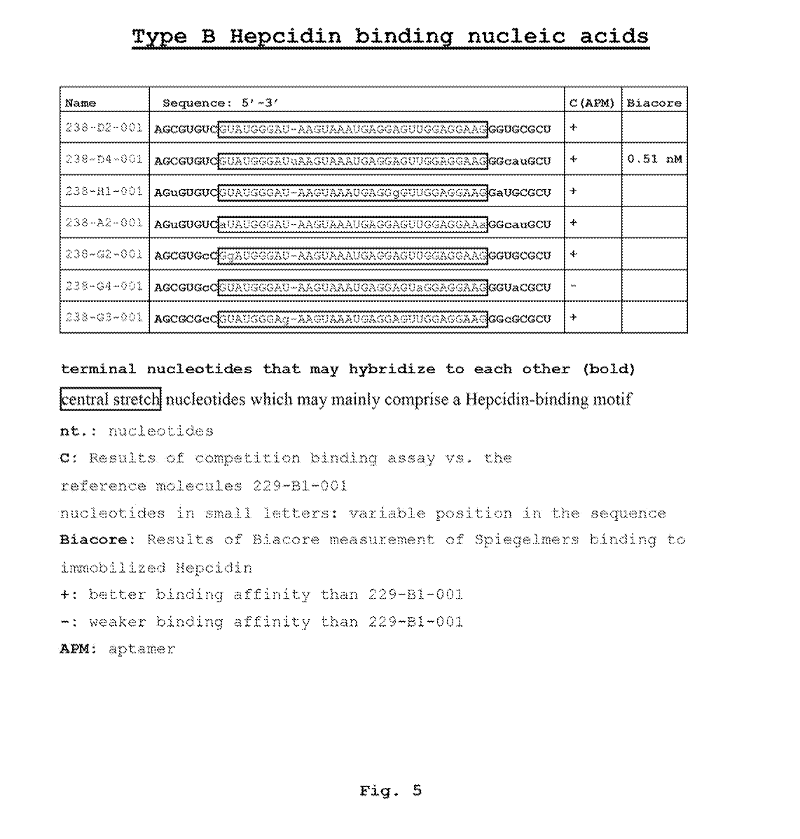

FIG. 5 shows an alignment of sequences of Type B hepcidin binding nucleic acids;

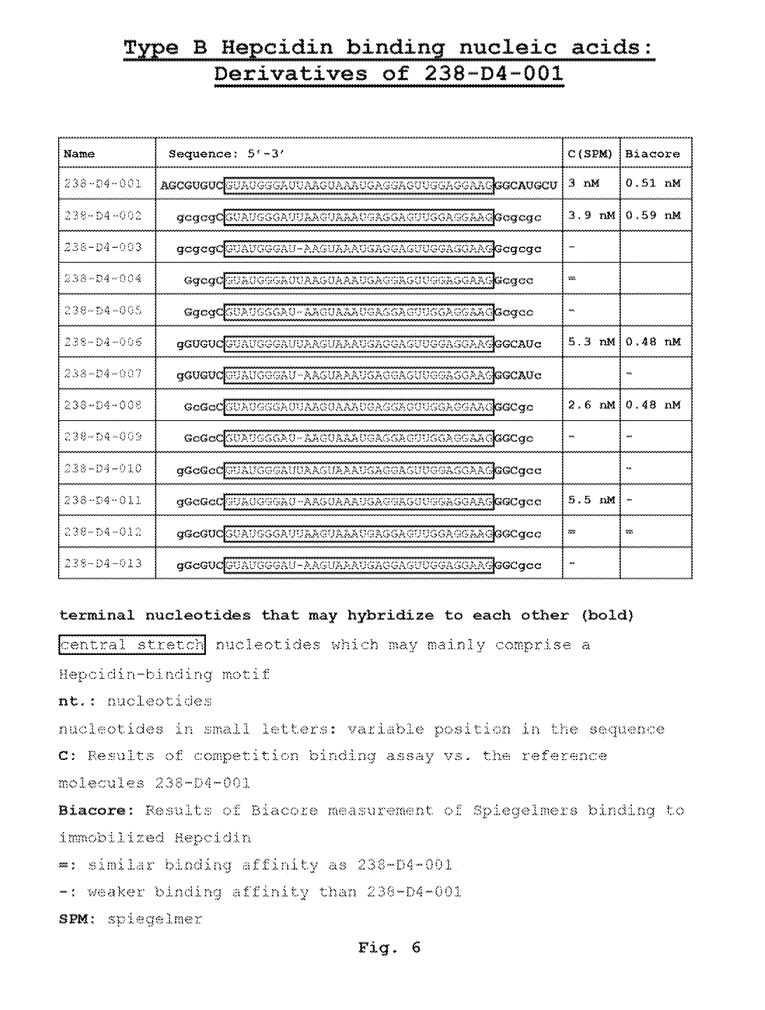

FIG. 6 shows derivatives of Type B hepcidin binding nucleic acid 238-D4-001;

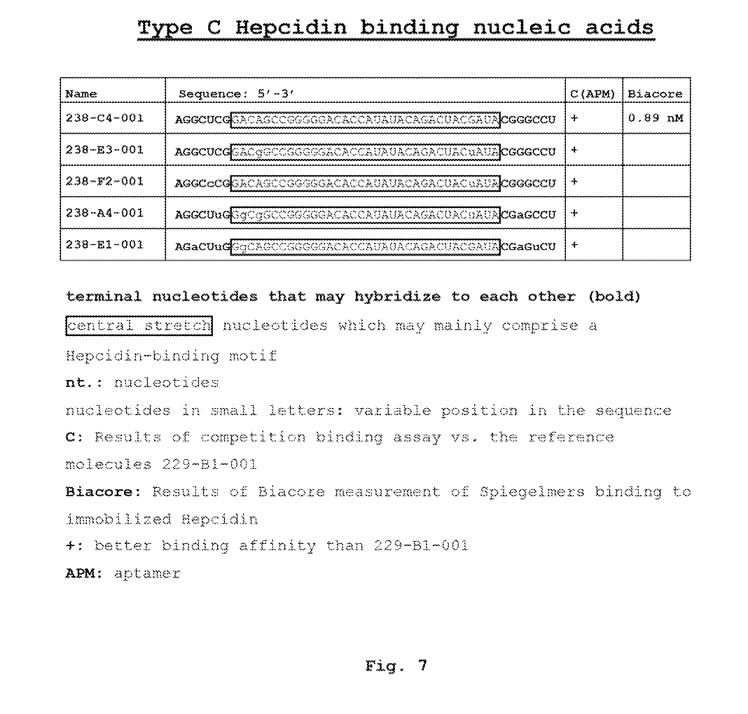

FIG. 7 shows an alignment of sequences of Type C hepcidin binding nucleic acids;

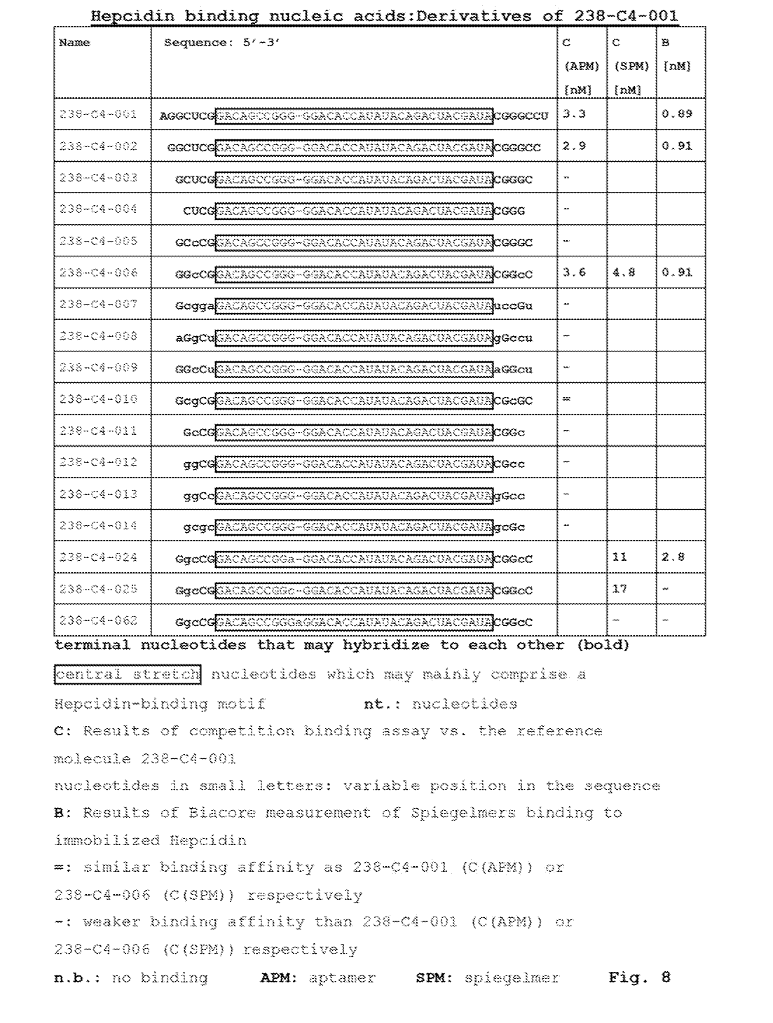

FIG. 8 shows derivatives of Type C hepcidin binding nucleic acid 238-C4-001;

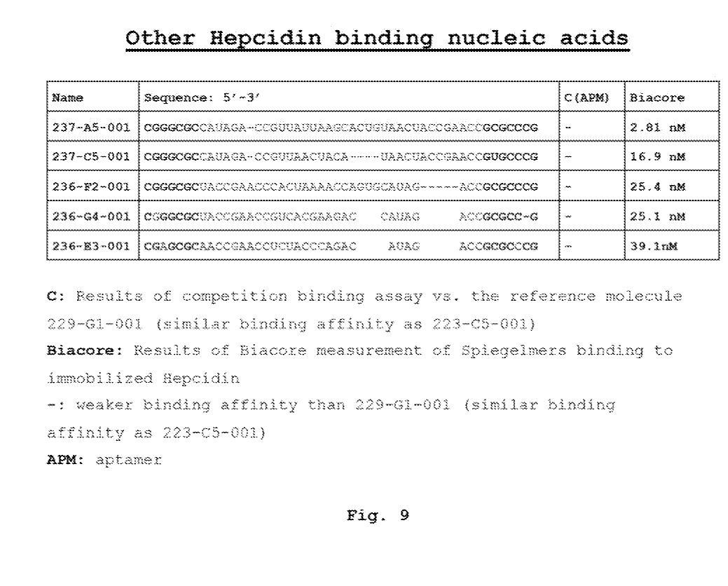

FIG. 9 shows an alignment of sequences of other hepcidin binding nucleic acids;

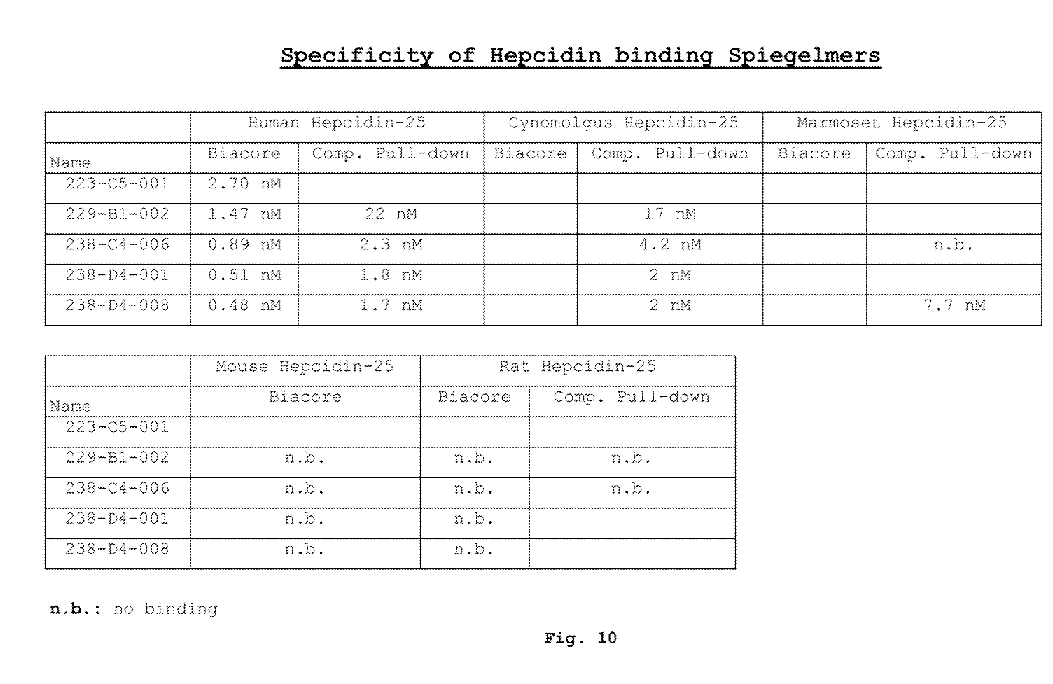

FIG. 10 shows data regarding the binding of hepcidin binding nucleic acids 223-C5-001, 229-B1-002, 238-C4-006, 238-D4-001 and 238-D4-008 to human hepcidin-25, cynomolgus hepcidin-25, marmoset hepcidin-25, mouse hepcidin-25 and rat hepcidin-25;

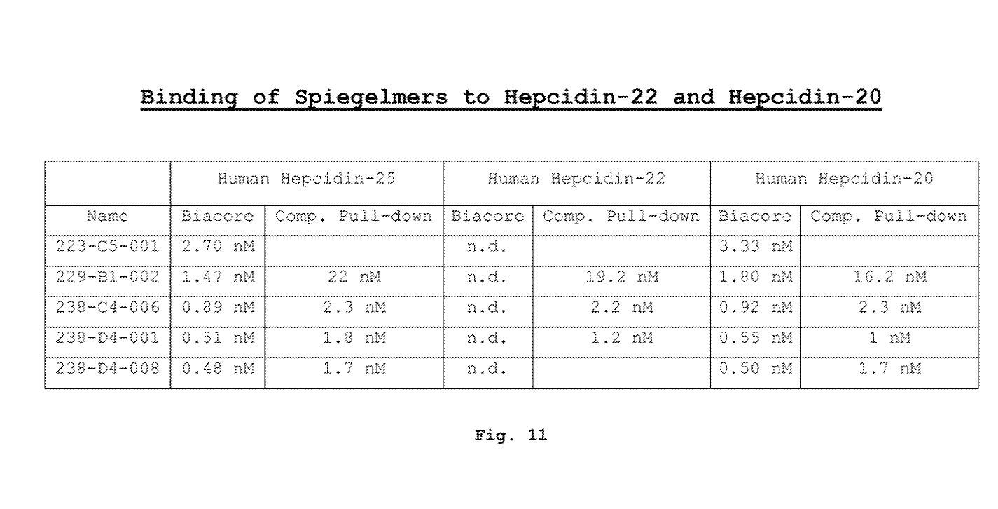

FIG. 11 shows data regarding the binding of hepcidin binding nucleic acids 223-C5-001, 229-B1-002, 238-C4-006, 238-D4-001 and 238-D4-008 to human hepcidin-25, hepcidin-22 and hepcidin-20;

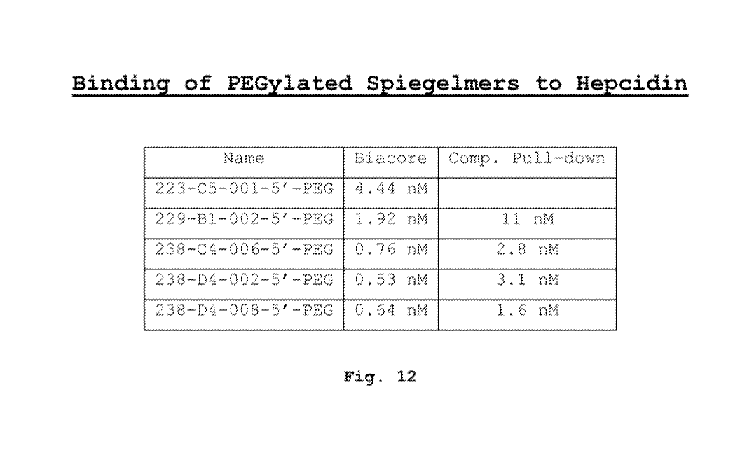

FIG. 12 shows data regarding the binding of hepcidin binding nucleic acids 223-C5-5′-PEG, 229-B1-002-5′-PEG, 238-C4-006-5′-PEG, 238-D4-002-5′-PEG and 238-D4-008-5′-PEG to human hepcidin-25;

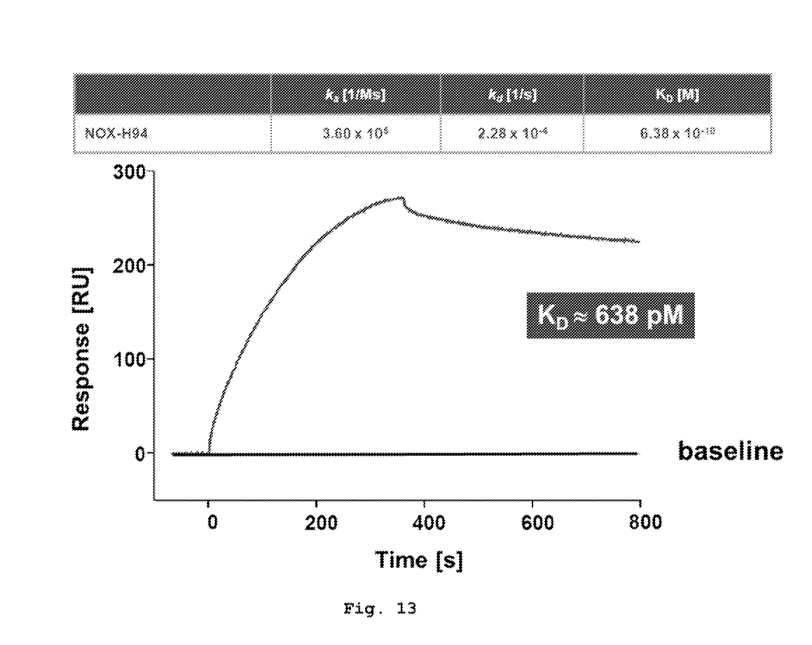

FIG. 13 shows Biacore 2000 sensorgram indicating the K_{D} value of the spiegelmer of hepcidin binding nucleic acid NOX-H94 (=238-D4-008-5′-PEG) binding to biotinylated human L-hepcidin at 37° C., whereby the biotinylated human L-hepcidin was immobilized by strepatavidin coupling procedure on a strepatavidin conjugated sensor chip at 37° C., represented as response (RU) over time;

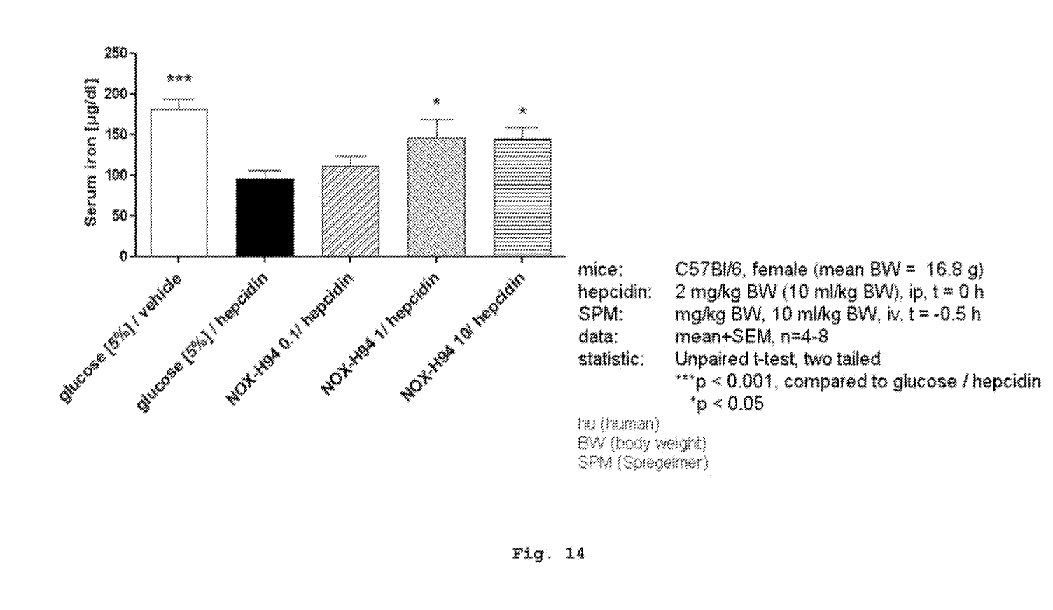

FIG. 14 shows the effect of the Spiegelmer NOX-H94 (=238-D4-008-5′-PEG) on hepcidin activity in vivo, whereby the decrease in serum iron caused by human hepcidin is completely blocked by application of Spiegelmer NOX-H94 (=238-D4-008-5′-PEG) prior to injection of human hepcidin;

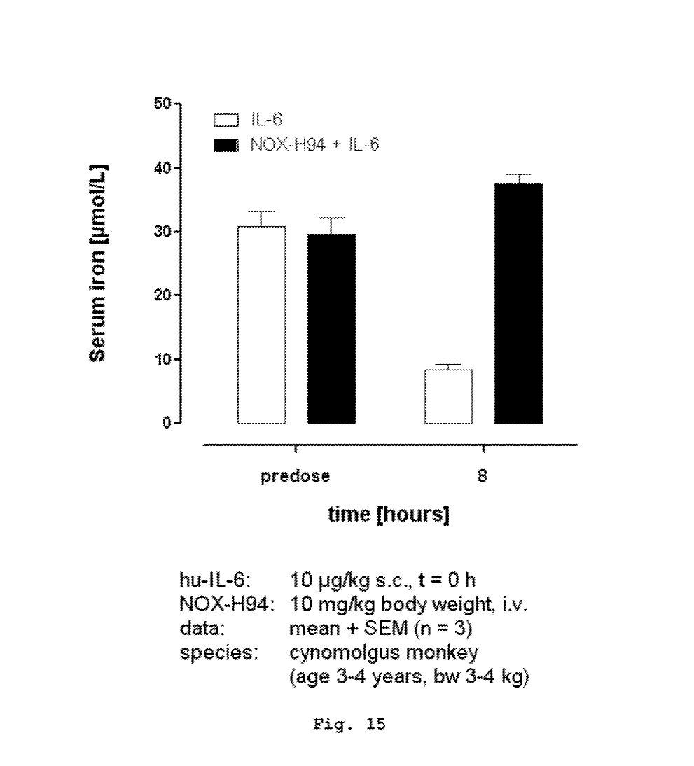

FIG. 15 shows the effect of the Spiegelmer NOX-H94 (=238-D4-008-5′-PEG) in an animal model (cynomolgus monkey) for anaemia of inflammation, whereby IL-6 induces hepcidin secretion subsequently resulting in anemia in non-human primates; within the experiment human IL-6 leads a reduction of serum iron concentration to 27% of the predose value of the vehicle/IL-6 treated monkeys, the decrease in serum iron is completely blocked by application of spiegelmer 238-D4-008-5′-PEG prior to injection of human IL-6;

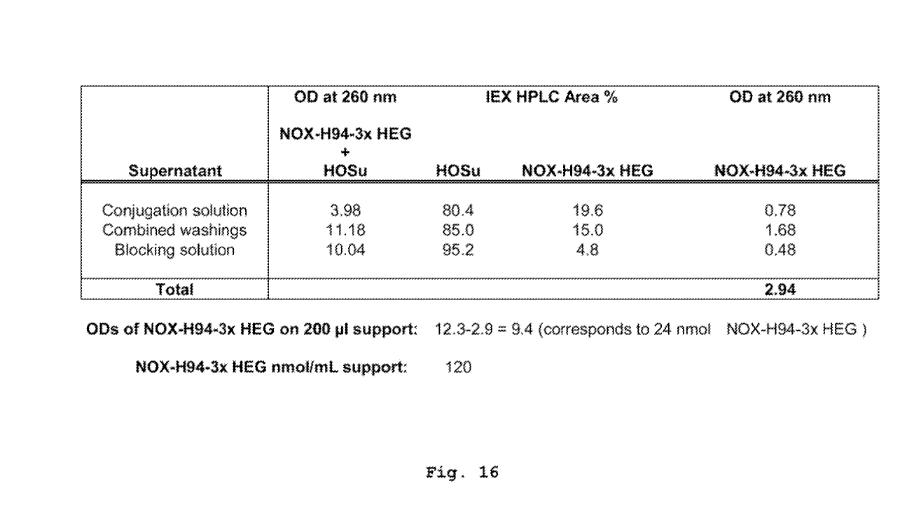

FIG. 16 shows the calculation of amount of hepcidin binding nucleic NOX-H94 3xHEG amino attached to different supports whereas the measured ODs are then resolved into contributions from N-Hydroxy succinimide (abbr. HOSu) and NOX-H94 3xHEG amino as determined by ion-exchange HPLC; the ODs from NOX-H94 3xHEG amino in the supernatants are calculated and added together to have the total ODs not bound to support; total ODs on support are then determined as is the loading on support;

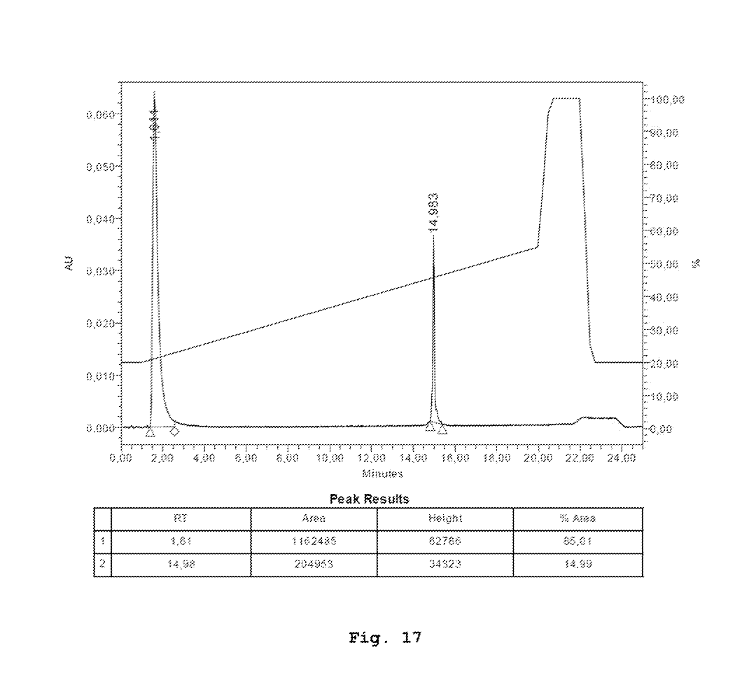

FIG. 17 shows an IEX chromatogram (absorbance at 260 nm) of combined supernatant of washes; N-Hydroxy succinimide (abbr. HOSu) and NOX-H94 3xHEG amino contributions to the OD amounts can be easily determined; in this example the nucleic acid molecules contributes 15% of the total ODs;

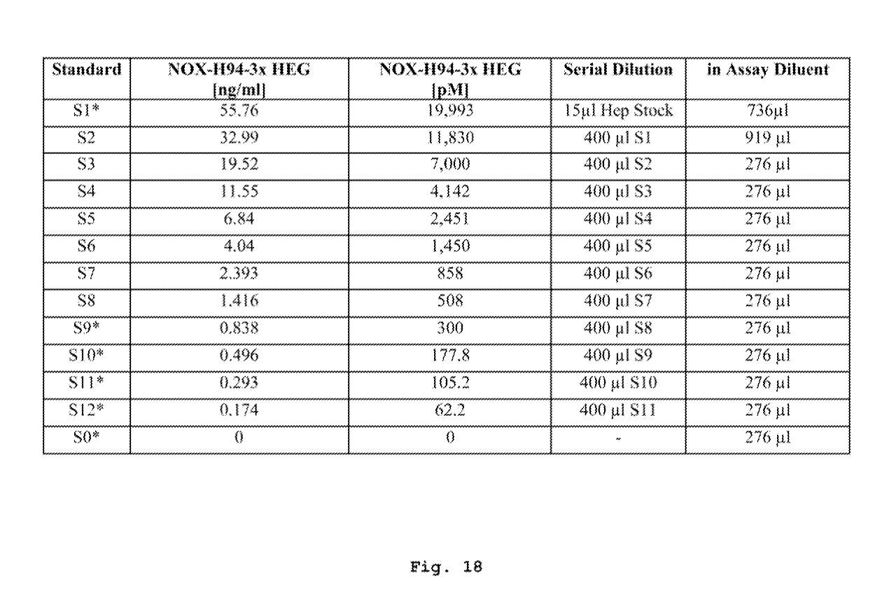

FIG. 18 shows dilution and pipetting scheme of standard calibration samples

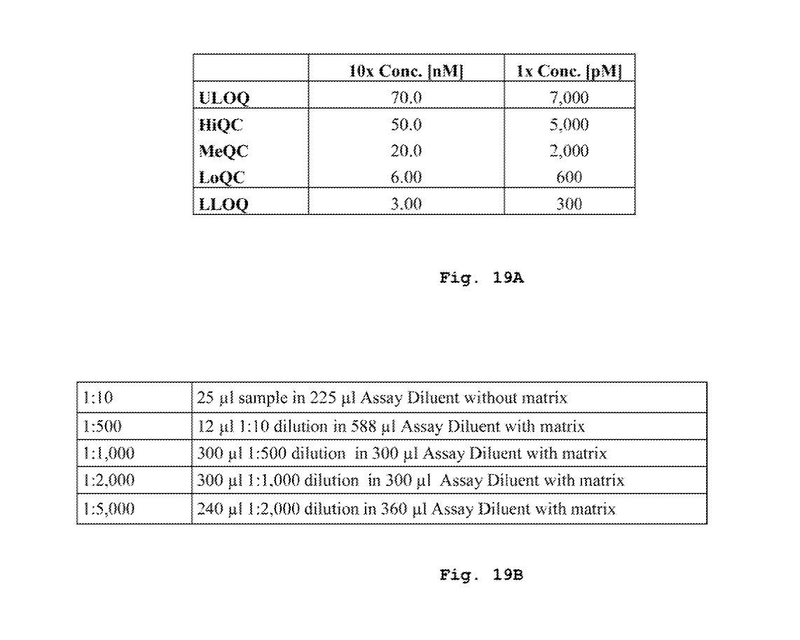

FIG. 19A shows quality control samples were prepared as 10 fold stock solutions and diluted likewise the test samples in the assay;

FIG. 19B shows the dilution and pipetting scheme for the test samples.

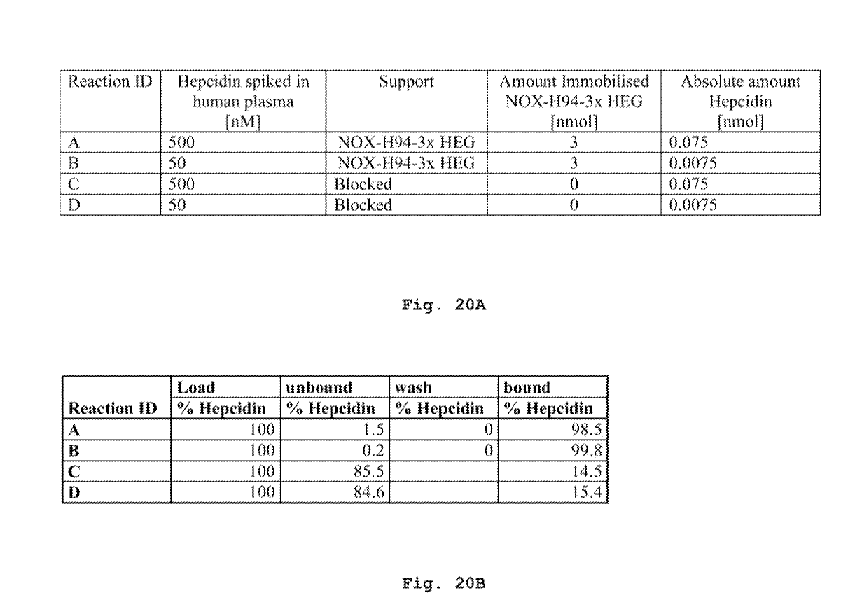

FIG. 20A shows shows an overview of incubation of the support used, wherein human pool plasma spiked with Hepcidin 25 was incubated with support containing immobilized NOX-H94 3xHEG or ethanolamine blocked sepharose support (no NOX-H94 3xHEG coupled, “Blocked”) at different concentrations; the relative amounts of hepcidin and NOX-H94 3xHEG are also specified; for incubation was used: 15 μl support+150 μl matrix, 2 h at room temperature;

FIG. 20B shows the determination of Hepcidin amounts that were determined by use of competitive Hepcidin ELISA (Bachem); “Load”=Hepcidin % amounts in Matrix before incubation with support; “Unbound”=Hepcidin % amounts in supernatant after incubation; “Wash”=Hepcidin % amounts in combined wash fractions.

### EXAMPLE 1

### Nucleic Acids that Bind Human Hepcidin

Using biotinylated human D-hepcidin-25 as a target, several nucleic acids that bind to human hepcidin, in particular human hepcidin-25, human hepcidin-22 and human hepcidin-20, could be generated; the nucleotide sequences of which are depicted in FIGS. 1 through 9. The nucleic acids were characterized on the aptamer, i. e. D-nucleic acid level using a direct pull-down assay (Example 3), a competitive pull-down assay (Example 3) and/or surface plasmon resonance measurement (Example 4) with biotinylated human D-hepcidin-25 or on the spiegelmer level, i. e. L-nucleic acid with the natural configuration of human hepcidin-25 (human L-hepcidin-25), in a competitive pull-down assay (Example 3), surface plasmon resonance measurement (Example 4), and/or an in vivo assay (Example 5 and 6). The spiegelmers and aptamers were synthesized as described in Example 2.

The nucleic acid molecules thus generated exhibit different sequence motifs, whereby three main types were identified and defined as Type A, Type B and Type C hepcidin binding nucleic acids and are depicted in FIGS. 1 through 8.

For definition of nucleotide sequence motifs, the IUPAC abbreviations for ambiguous nucleotides are used:

| | | |
|---|---|---|
| S | strong | G or C; |
| W | weak | A or U; |
| R | purine | G or A; |
| Y | pyrimidine | C or U; |
| K | keto | G or U; |
| M | imino | A or C; |
| B | not A | C or U or G; |
| D | not C | A or G or U; |
| H | not G | A or C or U; |
| V | not U | A or C or G; |
| N | all | A or G or C or U |

If not indicated to the contrary, any nucleic acid sequence or sequence of stretches and boxes, respectively, is indicated in the 5′→3′ direction.

### 1.1 Type A Hepcidin Binding Nucleic Acids

As depicted in FIG. 1, FIG. 2, FIG. 3 and FIG. 4 the Type A hepcidin binding nucleic acids comprise one central stretch of nucleotides, wherein the central stretch of nucleotides comprises at least two stretches of nucleotides—also referred to herein as boxes of nucleotides—defining a potential hepcidin binding motif: the first stretch of nucleotides Box A and the second stretch of nucleotides Box B.

The first stretch of nucleotides Box A and the second stretch of nucleotides Box B are linked to each other by a linking stretch of nucleotides.

Within the linking stretch of nucleotides some nucleotides can hybridize to each other, whereby upon hybridization a double-stranded structure is formed. However, such hybridization is not necessarily given in the molecule.

In general, Type A hepcidin binding nucleic acids comprise at their 5′-end and the 3′-end terminal stretches of nucleotides; the first-terminal stretch of nucleotides and the second terminal stretch of nucleotides. The first terminal stretch of nucleotides and the second terminal stretch of nucleotides can hybridize to each other, whereby upon hybridization a double-stranded structure is formed. However, such hybridization is not necessarily given in the molecule.

The five stretches of nucleotides of Type A hepcidin binding nucleic acids Box A, Box B, linking stretch of nucleotides, first terminal stretch of nucleotides and second terminal stretch of nucleotides can be differently arranged to each other: first terminal stretch of nucleotides—Box A—linking stretch of nucleotides—Box B—second terminal stretch of nucleotides or first terminal stretch of nucleotides—Box B—linking stretch of nucleotides—Box A—second terminal stretch of nucleotides.

However, the five stretches of nucleotides of Type A hepcidin binding nucleic acids Box A, Box B, linking stretch of nucleotides, first terminal stretch of nucleotides and second terminal stretch of nucleotides can be also arranged to each others as follows: second terminal stretch of nucleotides—Box A—linking stretch of nucleotides—Box B—first terminal stretch of nucleotides or second terminal stretch of nucleotides—Box B—linking stretch of nucleotides—Box A—first terminal stretch of nucleotides.

The sequences of the defined boxes or stretches of nucleotides may be different between the Type A hepcidin binding nucleic acids which influences the binding affinity to human hepcidin, in particular human hepcidin-25. Based on binding analysis of the different Type A hepcidin binding nucleic acids, the box A and B and their nucleotide sequences as described in the following are individually and more preferably in their entirety essential for binding to human hepcidin, in particular human hepcidin-25.

The Type A hepcidin binding nucleic acids according to the present invention are shown in FIGS. 1 to 4. All of them were tested as aptamers and or spiegelmers for their ability to bind human hepcidin-25, more precisely biotinylated human D-hepcidin-25 and biotinylated human L-hepcidin-24, respectively. The first Type A hepcidin binding nucleic acid that was characterized for its binding affinity to human hepcidin-25 is hepcidin binding nucleic acid 223-C5-001. The equilibrium binding constant K_{D} for human hepcidin-25 was determined by surface plasmon resonance measurement (K_{D}=2.7 nM determined with the spiegelmer sequence, FIG. 11). In addition to human hepcidin-25, hepcidin binding nucleic acid 223-C5-001 binds to human hepcidin-20 with almost the same binding affinity (FIG. 11).

The derivatives 223-C5-002, 223-C5-007 and 223-C5-008 of Type A hepcidin binding nucleic acid 223-C5-001 showed reduced binding affinity in a competitive pull-down assay in comparison to Type A hepcidin binding nucleic acid 223-C5-001 (FIG. 3). Indeed, hepcidin binding nucleic acid 223-C5-006 showed in the same assay format similar binding to human hepcidin-25 as 223-C5-001 (FIG. 3).

Type A hepcidin binding nucleic acids 223-B5-001, 223-A5-001, 223-A3-001, 223-F5-001, 223-G4-001, 223-A4-001, 229-C2-001, 229-B4-001, 229-E2-001, 229-G2-001, 229-G1-001, 229-C4-001, 229-A1-001, 238-E2-001, 223-A7-001, 236-G2-001, 236-D1-001, 229-D1-001 and 229-E1-001 were tested as aptamers in a competitive pull-down assay vs. Type A hepcidin binding nucleic acids 223-C5-001, whereby at first the binding affinity of the radioactively labeled aptamer 223-C5-001 was determined using the direct pull-down assay. No competition of the binding of Type A hepcidin binding nucleic acid 223-C5-001 by the nucleic acid 229-E1-001 could be observed (FIG. 2). This observation let assume that nucleic acid 229-E1-001 has no or very low binding affinity to human hepcidin-25. Type A hepcidin binding nucleic acids 223-B5-001, 223-A5-001, 223-A3-001, 223-A4-001, 229-C2-001, 229-B4-001, 229-E2-001, 229-C4-001, 238-E2-001, 223-A7-001, 236-G2-001 and 236-D1-001 showed reduced binding affinity in the competitive pull-down assay in comparison to Type A hepcidin binding nucleic acids 223-C5-001 (FIG. 1). Type A hepcidin binding nucleic acids 223-F5-001, 223-G4-001, 229-G1-001 and 229-D1-001 showed similar binding affinity as 223-C5-001 (FIGS. 1, 2). Better binding affinity for biotinylated human D-human hepcidin-25 could be observed for Type A hepcidin binding nucleic acids 229-B1-001 was further characterized. The equilibrium binding constant K_{D} of Type A hepcidin binding nucleic acids 229-B1-001 was determined by surface plasmon resonance measurement (K_{D}=1.25 nM determined with the spiegelmer sequence, data not shown).

The derivatives 229-B1-003, 229-B1-004, 229-B1-005 and 229-B1-006 of Type A hepcidin binding nucleic acids 229-B1-001 showed reduced binding affinity in a competitive pull-down assay in comparison to Type A hepcidin binding nucleic acids 229-B1-001 (FIG. 4). Indeed, Type A hepcidin binding nucleic acids 229-B1-002, 229-B1-007, 229-B1-008, 229-B1-009, 229-B1-010 and 229-B1-011 showed in the same assay format similar binding as or slightly improved binding to human hepcidin-25 in comparison to 229-B1-001 (FIG. 4). Type A hepcidin binding nucleic acids 229-B1-002 was further characterized. The equilibrium binding constant K_{D} of Type A hepcidin binding nucleic acid 229-B1-002 was determined by surface plasmon resonance measurement (K_{D}=1.47 nM determined with the a spiegelmer sequence. FIGS. 10 and 11).

Furthermore the binding specificity/selectivity of Type A hepcidin binding nucleic acid 229-B1-002 was tested with the following hepcidin molecules: human hepcidin-25, cynomolgus hepcidin-25, mouse hepcidin-25, rat hepcidin-25, human hepcidin-22, and human hepcidin-20 (FIGS. 10 and 11). Type A hepcidin binding nucleic acid 229-B1-002 shows similar binding to human hepcidin-25, cynomolgus hepcidin-25, human hepcidin-22, and human hepcidin-20 and no binding to mouse hepcidin-25 and rat hepcidin-25 (FIGS. 10 and 11).

Except for Type A nucleic acid 229-E1-001, all Type A hepcidin binding nucleic acids according to the present invention comprise the first stretch Box A. In Type A hepcidin binding nucleic acid 229-D1-001 Box A is linked with its 3′-end to the 5′-end of the second terminal stretch (FIG. 2). In all other Type A hepcidin binding nucleic acids Box A is linked with its 5′-end to the 3′-end of the first terminal stretch (FIG. 1 to 4). Type A hepcidin binding nucleic acids comprising the Box A share the sequence 5′ WAAAGUWGAR 3′ (SEQ.ID.NO. 188) for Box A. Beside Type A hepcidin binding nucleic acids 229-C4-001/236-G2-001 and 236-D1-001 that comprise a sequence of 5′ AAAAGUAGAA 3′ (SEQ.ID.NO. 200) AND 5′ AAAAGUUGAA 3′ (SEQ.ID.NO. 201), respectively, for Box A, the sequence of Box A of all other Type A hepcidin binding nucleic acids is 5′ UAAAGUAGAG 3′ (SEQ.ID.NO. 199).

Except for Type A hepcidin binding nucleic acid 236-D1-001 (see FIG. 2), all Type A hepcidin binding nucleic acids comprise a Box B with a sequence of 5′ RGMGUGWKAGUKC 3′ (SEQ.ID.NO. 189). Type A hepcidin binding nucleic acid 236-D1-001 comprise a Box B that is different from the consensus sequence of Box of the other Type A hepcidin binding nucleic acids 5′ GGGAUAUAGUGC 3′ (SEQ.ID.NO. 202). Because nucleic acid 229-E1-001 comprising no Box A does not or weakly bind to human hepcidin-25 as described supra, let assume, that beside Box B Box A is essential for binding to human hepcidin-25, in particular for high affinity binding to human hepcidin-25. In Type A hepcidin binding nucleic acid 229-D1-001 Box B is linked with its 5′-end to the 3′-end of the first terminal stretch (FIG. 2). In all other Type A hepcidin binding nucleic acids Box B—except for hepcidin binding nucleic acid 229-E1-001—is linked with its 3′-end to the 5′-end of the second terminal stretch (FIGS. 1, 3 and 4). Hepcidin binding nucleic acids with different sequences of Box B showed high binding affinity to human hepcidin-25.
a) 229-B1-001 and derivates, 223-C5-001 and derivatives, 223-B5-001, 223-A5-001, 223-A3-001, 223-F5-001, 223-G4-001, 223-A4-001, 238-E2: 5′ GGCGUGAUAGUGC 3′ (SEQ.ID.NO. 203);
b) 229-B4-001, 229-C2-001, 229-E2-001: 5′ GGAGUGUUAGUUC 3′ (SEQ.ID.NO. 204);
c) 229-G1-001: 5′ GGCGUGAGAGUGC 3′ (SEQ.ID.NO. 205);
d) 229-C4-001, 236-G2-001: 5′ AGCGUGAUAGUGC 3′ (SEQ.ID.NO. 206)
e) 238-A1-001: 5′ GGCGUGUUAGUGC 3′ (SEQ.ID.NO. 207)
f) 236-D1-001: 5′ GGGAUAUAGUGC 3′ (SEQ.ID.NO. 202).

Hepcidin binding nucleic acids that comprise Box A and Box B are linked to each other by a linking stretch of nucleotides of 10 to 18 nucleotides. The linking stretch of nucleotides comprises in 5′→3′ direction a first linking substretch of nucleotides, a second linking substretch of nucleotides and a third linking substretch of nucleotides, whereby preferably the first linking substretch of nucleotides and the third linking substretch of nucleotides optionally hybridize to each other, whereby upon hybridization a double-stranded structure is formed. However, such hybridization is not necessarily given in the molecule. If the nucleotides of the first linking substretch of nucleotides and third linking substretch of nucleotides hybridize to each other they are forming in between a loop of nucleotides (i.e. the second substretch) that do not hybridize to each other. The first substretch of nucleotides and the third substretch of nucleotides of the linking stretch of nucleotides of hepcidin binding nucleic acids comprise three (see 229-B1-001 and derivatives, 229-G1-001), four (see 223-C5-001 and derivatives, 223-B5-001, 223-A5-001, 223-A3-001, 223-F5-001, 223-G4-001, 223-A4-001, 229-C2-001, 229-B4-001, 229-E2-001, 238-A1-001, 238-E1-001, 237-A7-001), five (229-D1-001) or six (229-C4-001, 236-G2-001) nucleotides. Type A hepcidin binding nucleic acid 236-D1-001 comprises a linking stretch of nucleotides of 18 nucleotides, whereby due to the specific sequence of said linking stretch of nucleotides the linking stretch of nucleotides can not be classified in a first linking substretch of nucleotides, a second linking substretch of nucleotides and a third linking substretch of nucleotides.

As shown for hepcidin binding nucleic acids 223-C5-001 and derivatives thereof, 223-B5-001, 223-A5-001, 223-A3-001, 223-F5-001, 223-G4-001, 238-E2-001 and 223-A4-001 the first substretch of the linking stretch of nucleotides comprises the sequence of 5′ GGAC 3′ or 5′ GGAU 3′ or 5′ GGA 3′ and the third substretch of the linking stretch of nucleotides comprises the nucleotide sequence of 5′ GUCC 3′. Other combinations of the first and the third substretch of the linking stretch of nucleotides are
a) 5′ GCAG 3′ and 5′ GUGC 3′ (229-C2-001, 229-B4-001, 229-E2-001, 237-A7-001) or
b) 5′ GAC 3′ and 5′ GUC 3′ (229-B1-001 and derivatives thereof, 229-G1-001) or
c) 5′ ACUUGU 3′ and 5′ GCAAGU 3′ (229-C4-001) or
d) 5′ ACUUGU 3′ and 5′ GCAAGC 3′ (236-G2-001) or
e) 5′ UUCAG 3′ and 5′ CUGGA 3′ (229-D1-001) or
f) 5′ GGGC 3′ and 5′ GCCC 3′ (238-A1-001).

As shown in FIGS. 1, 2, 3 and 4 the second substretch of the linking stretch of nucleotides comprises three to five nucleotides, whereby the different sequences are very heterogeneous: 5′ CGAAA 3′, 5′ GCAAU 3′, 5′ GUAAU 3′, 5′ AAUU 3′, 5′ AUAAU 3′, 5′ AAUA 3′, 5′ CCA 3′, 5′ CUA 3′, 5′ UCA 3′, 5′ ACA 3′, 5′ GUU 3′, 5′ UGA 3′and 5′ GUA 3′. The second substretch of the linking stretch of nucleotides of hepcidin binding nucleic acids can be summarized into the following generic sequences: 5′ VBAAW 3′, 5′ AAUW 3′ or 5′ NBW 3′.

However, the hepcidin binding nucleic acids with the best binding affinity comprise the following sequences for the second substretch of the linking stretch of nucleotides:
a) 5′ AAUU 3′ (229-B1 and derivatives thereof)
b) 5′ CCA 3′ (223-C5 and derivatives thereof)
c) 5′ CUA 3′ (223-F5-001)
d) 5′ UCA 3′ (223-G4-001)
e) 5′ AAUA 3′ (229-G1-001).

As described supra, the nucleotide sequence of the first and the third substretch of the linking stretch are related to each other. Moreover, the nucleotide sequence of the second substretch of the linking stretch of nucleotides is related to a specific pair of the first and the third substretch of nucleotides leading to the following sequences or generic sequences of the linking stretch of nucleotides of hepcidin binding nucleic acids:
a) 5′ GGACBYAGUCC 3′ (SEQ.ID.NO. 208) (223-C5-001, 223-C5-002, 223-C5-006, 223-C5-007, 223-B5-001, 223-A5-001, 223-A3-001, 223-F5-001, 223-G4-001, 238-E2-001), preferably 5′ GGACCCAGUCC 3′ (SEQ.ID.NO. 193), 5′ GGACCUAGUCC 3′ (SEQ.ID.NO. 194) OR 5′ GGACUCAGUCC 3′ (SEQ.ID.NO. 195) or 5′ GGACGUAGUCC 3′ (SEQ.ID.NO. 214), more preferably 5′ GGACCCAGUCC 3′ (SEQ.ID.NO. 193), 5′ GGACCUAGUCC 3′ (SEQ.ID.NO. 194) or 5′ GGACUCAGUCC 3′ (SEQ.ID.NO. 195); or
b) 5′ GGAUACAGUCC 3′ (SEQ.ID.NO. 209)(223-A4-001); or
c) 5′ GCAGGYAAUCUGC 3′ (SEQ.ID.NO. 210) (229-C2-001, 229-B4-001, 229-E2-001), preferably 5′ GCAGGUAAUCUGC 3′ (SEQ.ID.NO. 196) or 5′ GCAGGCAAUCUGC 3′ (SEQ.ID.NO. 197), more preferably 5′ GCAGGUAAUCUGC 3′ (SEQ.ID.NO. 196); or
d) 5′ GACAAUWGUC 3′ (SEQ.ID.NO. 211) (229-B1-001 and derivatives 229-G1-001), preferably 5′ GACAAUUGUC 3′ (SEQ.ID.NO. 198) or 5′ GACAAUAGUC 3′ (SEQ.ID.NO. 157); or
e) 5′ ACUUGUCGAAAGCAAGY 3′ (SEQ.ID.NO. 212)(229-C4-001, 236-G2-001); or
f) 5′ UCCAGGUUCUGGA 3′ (SEQ.ID.NO. 109)(229-D1-001); or
g) 5′ GGGCUGAGCCC 3′ (SEQ.ID.NO. 190)(238-A1-001); or
h) 5′ GCAGAUAAUCUGC 3′ (SEQ.ID.NO. 191)(237-A7-001); or
i) 5′ GGACCAGUCC 3′ (SEQ.ID.NO. 192)(223-C5-008).

As mentioned before, the linking stretch of nucleotides of Type A hepcidin binding nucleic acid 236-D1-001 can not be classified in a first linking substretch of nucleotides, a second linking substretch of nucleotides and a third linking substretch of nucleotides. However, the sequence of the linking stretch of nucleotides of Type A hepcidin binding nucleic acid 236-D1-001 is 5′ AUUUGUUGGAAUCAAGCA 3′ (SEQ.ID.NO. 215).

The first and second terminal stretches of nucleotides of Type A hepcidin binding nucleic acids comprise four (e.g. 229-C4-001), five, (e.g. 223-C5-007), six (e.g. 229-B1-001) or seven (e.g. 223-C5-001) nucleotides, whereby the stretches optionally hybridize with each other, whereby upon hybridization a double-stranded structure is formed. This double-stranded structure can consists of four to seven basepairs. However, such hybridization is not necessarily given in the molecule.

Combining the first and second terminal stretches of nucleotides of all tested hepcidin binding nucleic acids the generic formula for the first terminal stretch of nucleotides and for the second terminal stretch of nucleotides are 5′X₁X₂X₃BKBK 3′ (first terminal stretch of nucleotides) and 5′MVVVX₄X₅X₆ 3′ (second terminal stretch of nucleotides), whereby
X₁ is G or absent, X₂ is S or absent, X₃ is V or absent, X₄ is B or absent, X₅ is S or absent, and X₆ is C or absent,
   preferably
a) X₁ is G, X₂ is S, X₃ is V, X₄ is B, X₅ is S, and X₆ is C or
b) X₁ is absent, X₂ is S, X₃ is V, X₄ is B, X₅ is S, and X₆ is C or
d) X₁ is G, X₂ is S, X₃ is V, X₄ is B, X₅ is S, and X₆ is absent or
e) X₁ is absent, X₂ is S, X₃ is V, X₄ is B, X₅ is S, and X₆ is absent or
f) X₁ is absent, X₂ is absent, X₃ is V, X₄ is B, X₅ is S, and X₆ is absent or
g) X₁ is absent, X₂ is S, X₃ is V, X₄ is B, X₅ is absent, and X₆ is absent or
f) X₁ is absent, X₂ is absent, X₃ is V or absent, X₄ is B or absent, X₅ is absent, and X₆ is absent.

However, the hepcidin binding nucleic acids with the best binding affinity comprise the following combinations of first and second terminal stretches of nucleotides:
a) 223-C5-001, 223-F5-001, 223-G4-001: 5′ GCACUCG 3′ (first terminal stretch of nucleotides) and 5′ CGAGUGC 3′ (second terminal stretch of nucleotides);
b) 229-B1-002: 5′ GCUGUG 3′ (first terminal stretch of nucleotides) and 5′ CACAGC 3′ (second terminal stretch of nucleotides);
c) 229-B1-001, 229-G1-001: 5′ CGUGUG 3′ (first terminal stretch of nucleotides) and 5′ CACACG 3′ (second terminal stretch of nucleotides);
d) 229-D1-001: 5′ CGUGCU 3′ (first terminal stretch of nucleotides) and 5′ AGCACG 3′ (second terminal stretch of nucleotides);
e) 223-C5-006: 5′ CGCGCG 3′ (first terminal stretch of nucleotides) and 5′ CGCGCG 3′ (second terminal stretch of nucleotides)
f) 229-B1-007: 5′ GCCGUG 3′ (first terminal stretch of nucleotides) and 5′ CACGGC 3′ (second terminal stretch of nucleotides)
g) 229-B1-008: 5′ GCGGUG 3′ (first terminal stretch of nucleotides) and 5′ CACCGC 3′ (second terminal stretch of nucleotides)
h) 229-B1-009: 5′ GCUGCU 3′ (first terminal stretch of nucleotides) and 5′ CGCAGC 3′ (second terminal stretch of nucleotides)
i) 229-B1-010: 5′ GCUGGG 3′ (first terminal stretch of nucleotides) and 5′ CCCAGC 3′ (second terminal stretch of nucleotides)
j) 229-B1-011: 5′ GCGGCG 3′ (first terminal stretch of nucleotides) and 5′ CGCCGC 3′ (second terminal stretch of nucleotides).

In order to prove the functionality of hepcidin binding nucleic acids as spiegelmers, Type A hepcidin binding nucleic acids 223-C5-001 and 229-B1-002 were synthesized as spiegelmers comprising an Amino-group at its 5′-end. To the amino-modified spiegelmers 223-C5-001-5′-Amino and 229-B1-002-5′-Amino a 40 kDa PEG-moiety was coupled leading to Type A hepcidin binding nucleic acids 223-C5-001-5′-PEG and 229-B1-002-5′-PEG. Synthesis and PEGyation of the spiegelmer is described in Example 2.

The equilibrium binding constant K_{D} of spiegelmers 223-C5-001-5′-PEG and 229-B1-002 were determined by surface plasmon resonance measurement (FIG. 12);
223-C5-001-5′-PEG; K_{D}=4.44 nM;
229-B1-002-5′-PEG; K_{D}=1.92 nM.

The spiegelmer 223-C5-001-5′-PEG was tested to inhibit/antagonize the function of hepcidin in vivo. The applicability for in vivo use of the Spiegelmer 223-C5-001-5′-PEG was tested in an animal model for anaemia of inflammation, wherein the known properties of human hepcidin-25 to induce a serum iron decrease was utilized (Example 5).

### 1.2 Type B Hepcidin Binding Nucleic Acids

As depicted in FIGS. 5 and 6, the Type B hepcidin binding nucleic acids comprise one central stretch of nucleotides defining a potential hepcidin binding motif.

In general, Type B hepcidin binding nucleic acids comprise at their 5′-end and the 3′-end terminal stretches of nucleotides; the first terminal stretch of nucleotides and the second terminal stretch of nucleotide. The first terminal stretch of nucleotides and the second terminal stretch of nucleotides can hybridize to each other, whereby upon hybridization a double-stranded structure is formed. However, such hybridization is not necessarily given in the molecule.

The three stretches of Type B hepcidin binding nucleic acids the first terminal stretch of nucleotides, the central stretch of nucleotides and the second terminal stretch of nucleotides can be differently arranged to each other: first terminal stretch of nucleotides—central stretch of nucleotides—second terminal stretch of nucleotides or second terminal stretch of nucleotides—central stretch of nucleotides—first terminal stretch of nucleotides.

The sequences of the defined stretches may be different between the Type B hepcidin binding nucleic acids which influences the binding affinity to human hepcidin, in particular human hepcidin-25. Based on binding analysis of the different hepcidin binding nucleic acids, the central stretch of nucleotides and its nucleotide sequences as described in the following is individually and more preferably in its entirety essential for binding to human hepcidin-25

The Type B hepcidin binding nucleic acids according to the present invention are shown in FIGS. 5 and 6. All of them were tested as aptamers or spiegelmers for their ability to bind human hepcidin-25, more precisely biotinylated human D-hepcidin-25 and biotinylated human L-hepcidin-25, respectively.

The Type B hepcidin binding nucleic acids 238-D2-001, 238-D4-001, 238-H1-001, 238-A2-001, 238-G2-001, 238-G4-001, 238-G3-001 were tested as aptamers in a competitive pull-down assay vs. Type A hepcidin binding nucleic acid 229-B1-001. Only Type B hepcidin binding nucleic acids 238-G4-001 showed reduced binding affinity in the competitive pull-down assay in comparison to Type A hepcidin binding nucleic acid 229-B1-001 (FIG. 5). Type B hepcidin binding nucleic acids 238-D2-001, 238-D4-001, 238-H1-001, 238-A2-001, 238-G2-001 and 238-G3-001 showed improved binding affinity in comparison to Type A hepcidin binding nucleic acid 229-B1-001 (FIG. 5). Type B hepcidin binding nucleic acid 238-D4-001 was further characterized. The equilibrium binding constant K_{D} of spiegelmer 238-D4-001 was determined by surface plasmon resonance measurement (K_{D}=0.51 nM; FIG. 5).

The derivatives 238-D4-003, 238-D4-005, 238-D4-007, 238-D4-009, 238-D4-010, 238-D4-011, and 238-D4-013 of Type B hepcidin binding nucleic acids 238-D4-001 showed reduced binding affinity in a competitive pull-down assay (or shown by surface plasmon resonance measurement) in comparison to Type B hepcidin binding nucleic acid 238-D4-001 (FIG. 6). Indeed, hepcidin binding nucleic acids 238-D4-002, 238-D4-004, 238-D4-006, 238-D4-008 and 238-D4-012 showed in the same assay format similar binding to human hepcidin as 238-D4-001 (FIG. 6). The equilibrium binding constant K_{D} of spiegelmers 238-D4-002, 238-D4-006 and 238-D4-008 were determined by surface plasmon resonance measurement. The calculated equilibrium binding constants of the derivatives of 238-D4-001 are in same range as shown for 238-D4-001 itself (FIG. 6).

Furthermore the binding selectivity of Type B hepcidin binding nucleic acids 238-D4-001 and 238-D4-008 were tested with the following hepcidin molecules: human hepcidin-25, cynomolgus hepcidin-25, marmoset hepcidin-25 (only for 238-D4-008), mouse hepcidin-25, rat hepcidin-25, human hepcidin-22 (not for 238-D4-008) and human hepcidin-20 (FIGS. 10 and 11). Type B hepcidin binding nucleic acid 238-D4-001 and 238-D4-008 shows similar binding to human hepcidin-25, human hepcidin-22, human hepcidin-20 and cynomolgus hepcidin-25, weaker binding to marmoset hepcidin-25 and no binding to mouse hepcidin-25 and rat hepcidin-25, (FIGS. 10 and 11).

The Type B hepcidin binding nucleic acids according to the present invention share the sequence 5′ RKAUGGGAKUAAGUAAAUGAGGRGUWGGAGGAAR 3′ (SEQ.ID.NO. 182) or 5′ RKAUGGGAKAAGUAAAUGAGGRGUWGGAGGAAR 3′ (SEQ.ID.NO. 183) for the central stretch of nucleotides. Type B hepcidin binding nucleic acid 238-D4-001 and its derivatives that showed the same binding affinity to human hepcidin-25 share the consensus sequence comprises the sequence 5′ GUAUGGGAUUAAGUAAAUGAGGAGUUGGAGGAAG 3′ (SEQ.ID.NO. 184) for the central stretch of nucleotides.

The first and second terminal stretches of nucleotides of Type B hepcidin binding nucleic acids comprise five (238-D4-004, 238-D4-005, 238-D4-008, 238-D4-009), six (238-D4-002, 238-D4-003, 238-D4-006, 238-D4-007, 238-D4-010, 238-D4-011, 238-D4-012, 238-D4-013) or eight (238-D2-001, 238-D4-001, 238-H1-001, 238-A2-001, 238-G2-001, 238-G4-001, 238-G3-001) nucleotides, whereby the stretches optionally hybridize with each other, whereby upon hybridization a double-stranded structure is formed. This double-stranded structure can consists of five to eight basepairs. However, such hybridization is not necessarily given in the molecule.

Combining the first and second terminal stretches of nucleotides of all tested Type B hepcidin binding nucleic acids the generic formula for the first terminal stretch of nucleotides and for the second terminal stretch of nucleotides are 5′ X₁X₂X₃SBSBC 3′ (first terminal stretch of nucleotides) and 5′ GVBVB₄X₅X₆ 3′ (second terminal stretch of nucleotides), wherein X₁ is A or absent, X₂ is G or absent, X₃ is B or absent, X₄ is S or absent, X₅ is C or absent, and X₆ is U or absent,

preferably
a) X₁ is A, X₂ is G, X₃ is B, X₄ is S, X₅ is C, and X₆ is U or
b) X₁ is absent, X₂ is G, X₃ is B, X₄ is S, X₅ is C, and X₆ is U or
c) X₁ is G, X₂ is G, X₃ is B, X₄ is S, X₅ is C, and X₆ is absent or
d) X₁ is absent, X₂ is G, X₃ is B, X₄ is S, X₅ is C, and X₆ is absent or
e) X₁ is absent, X₂ is absent, X₃ is B, X₄ is S, X₅ is C, and X₆ is absent or
f) X₁ is absent, X₂ is G, X₃ is B, X₄ is S, X₅ is absent, and X₆ is absent or
g) X₁ is absent, X₂ is absent, X₃ is B or absent, X₄ is S or absent, X₅ is absent, and X₆ is absent.

However, the best binding Type B hepcidin binding nucleic acids comprise the following combinations of first and second terminal stretches of nucleotides:
a) 238-D2-001: 5′ AGCGUGUC 3′ (first terminal stretch of nucleotides) and 5′ GGUGCGCU 3′ (second terminal stretch of nucleotides).
b) 238-D4-001: 5′ AGCGUGUC 3′ (first terminal stretch of nucleotides) and 5′ GGCAUGCU 3′ (second terminal stretch of nucleotides).
c) 238-H1-001: 5′ AGUGUGUC 3′ (first terminal stretch of nucleotides) and 5′ GAUGCGCU 3′ (second terminal stretch of nucleotides).
d) 238-A2-001: 5′ AGUGUGUC 3′ (first terminal stretch of nucleotides) and 5′ GGCAUGCU 3′ (second terminal stretch of nucleotides).
e) 238-G2-001: 5′ AGCGUGCC 3′ (first terminal stretch of nucleotides) and 5′ GGUGCGCU 3′ (second terminal stretch of nucleotides).
f) 238-G3-001: 5′ AGCGCGCC 3′ (first terminal stretch of nucleotides) and 5′ GGCGCGCU 3′ (second terminal stretch of nucleotides).
g) 238-D4-002: 5′ GCGCGC 3′ (first terminal stretch of nucleotides) and 5′ GCGCGC 3′ (second terminal stretch of nucleotides)
h) 238-D4-006: 5′ GGUGUC 3′ (first terminal stretch of nucleotides) and 5′ GGCAUC 3′ (second terminal stretch of nucleotides)
i) 238-D4-012: 5′ GGCGUC 3′ (first terminal stretch of nucleotides) and 5′ GGCGCC 3′ (3′-terminal stretch of nucleotides)
j) 238-D4-008: 5′ GCGCC 3′ (first terminal stretch of nucleotides) and 5′ GGCGC 3′ (second terminal stretch of nucleotides)
k) 238-D4-004: 5′ GGCGC 3′ (first terminal stretch of nucleotides) and 5′ GCGCC 3′ (second terminal stretch of nucleotides)

In order to prove the functionality of Type B hepcidin binding nucleic acids as spiegelmers, hepcidin binding nucleic acids 238-D4-002 and 238-D4-008 were synthesized as spiegelmer comprising an Amino-group at its 5′-end. To the amino modified spiegelmers 238-D4-002-5′-Amino and 237-D4-008-5′-Amino a 40 kDa PEG-moiety was coupled leading to hepcidin binding nucleic acids 238-D4-002-5′-PEG and 238-D4-008-5′PEG. Synthesis and PEGyation of the spiegelmer is described in Example 2.

The equilibrium binding constant K_{D} of spiegelmers 238-D4-002-┘-PEG and 238-D4-008-5′-PEG were determined by surface plasmon resonance measurement (FIG. 12):
238-D4-002-5′-PEG: 0.53 nM,
238-D4-008-5′-PEG: 0.64 nM.

The spiegelmer 238-D4-008-5′-PEG was tested to inhibit/antagonize the function of hepcidin in vivo. The applicability for in vivo use of the spiegelmer 238-D4-008-5′-PEG was tested in an animal model for anaemia of inflammation, wherein the known properties of human hepcidin-25 to induce a serum iron decrease was utilized (Example 5, FIG. 14). Moreover, Spiegelmer 238-D4-008-5′-PEG was tested in another animal model (cynomolgus monkey) for anaemia of inflammation, whereby IL-6 induces hepcidin secretion subsequently resulting in anemia in non-human primates. Within the experiment human IL-6 leads a reduction of serum iron concentration (Example 6, FIG. 15).

### 1.3 Type C Hepcidin Binding Nucleic Acids

As depicted in FIGS. 7 and 8 the Type C hepcidin binding nucleic acids comprise one central stretch of nucleotides defining a potential hepcidin binding motif.

In general, Type C hepcidin binding nucleic acids comprise at their 5′-end and the 3′-end terminal stretches: the first terminal stretch of nucleotides and the second terminal stretch of nucleotides. The first terminal stretch of nucleotides and the second terminal stretch of nucleotides can hybridize to each other, whereby upon hybridization a double-stranded structure is formed. However, such hybridization is not necessarily given in the molecule.

The three stretches of nucleotides of Type C hepcidin binding nucleic acids first terminal stretch of nucleotides, central stretch of nucleotides and second terminal stretch of nucleotides can be differently arranged to each other: first terminal stretch of nucleotides—central stretch of nucleotides—second terminal stretch of nucleotides or second terminal stretch of nucleotides—central stretch of nucleotides—first terminal stretch of nucleotides.

The sequences of the defined stretches may be different between the Type C hepcidin binding nucleic acids which influences the binding affinity to human hepcidin, in particular human-hepcidin-25. Based on binding analysis of the different Type C hepcidin binding nucleic acids, the central stretch of nucleotides and its nucleotide sequences as described in the following is individually and more preferably in its entirety essential for binding to human hepcidin.

Type C hepcidin binding nucleic acids according to the present invention are shown in FIGS. 7 and 8. All of them were tested as aptamers or spiegelmers for their ability to bind human hepcidin-25, more precisely biotinylated human D-hepcidin-25 and biotinylated human L-hepcidin-25.

The Type C hepcidin binding nucleic acids 238-C4-001, 238-E3-001, 238-F2-001, 238-A4-001 and 238-E1-001 were tested as aptamers in a competitive pull-down assay vs. Type A hepcidin binding nucleic acid 229-B1-001. The Type C hepcidin binding nucleic acids showed improved binding affinity in comparison to Type C hepcidin binding nucleic acid 229-B1-001 (FIG. 7). Type C hepcidin binding nucleic acid 238-C4-001 was further characterized. The equilibrium binding constant K_{D} of the spiegelmer 238-C4-001 was determined by surface plasmon resonance measurement (K_{D}=0.9 nM; FIG. 7).

The derivatives 238-C4-003, 238-C4-004, 238-C4-005, 238-C4-007, 238-C4-008, 238-C4-009, 238-C4-011, 238-C4-012, 238-C4-013, 238-C4-014, 238-C4-024, 238-C4-025 and 238-C4-062 of Type C hepcidin binding nucleic acid 238-C4-001 showed reduced binding affinity in a competitive pull-down assay or by plasmon resonance measurement in comparison to hepcidin binding nucleic acid 238-C4-001 or 238-C4-006 (FIG. 8). Indeed, hepcidin binding nucleic acids 238-C4-002, 238-C4-006 and 238-C4-010 showed in the same assay similar binding to human hepcidin-25 as 238-C4-001 (FIG. 8). The equilibrium binding constant K_{D} or Spiegelmers 238-C4-002 and 238-C4-006 were determined by surface plasmon resonance measurement. The calculated equilibrium binding consists of the derivatives of 238-C4-001 are in same range as shown for 238-C4-001 itself (FIG. 8).

Furthermore the binding specificity/selectivity of Type C hepcidin binding nucleic acid 238-C4-006 was tested with the following hepcidin molecules: human hepcidin-25, cynomolgus hepcidin-25, marmoset hepcidin-25, mouse hepcidin-25, rat hepcidin-25, human hepcidin-22 and human hepcidin-20 (FIGS. 10 and 11). Type C hepcidin binding nucleic acid 238-C4-006 shows similar binding to human hepcidin-25, human hepcidin-22, human hepcidin-20 and cynomolgus hepcidin-25 and no binding to marmoset hepcidin-25, mouse hepcidin-25 and rat hepcidin-25 (FIGS. 10 and 11).

Type C hepcidin binding nucleic acids according to the present invention share the sequence 5′ GRCRGCCGGVGGACACCAUAUACAGACUACKAUA 3′ (SEQ.ID.NO. 185) or 5′ GRCRGCCGGVAGGACACCAUAUACAGACUACKAUA 3′ (SEQ.ID.NO. 186) for the central stretch of nucleotides. Type C hepcidin binding nucleic acid 238-C4-001 and its derivatives 238-C4-002, 238-C4-005, 238-C4-010 and Type C hepcidin binding nucleic acids 238-E3-001, 238-F2-001, 238-A4-001, 238-E1-001 that all showed the same binding affinity share the consensus sequence 5′ GRCRGCCGGGGGACACCAUAUACAGACUACKAUA 3′ (SEQ.ID.NO. 216), and preferably the sequence 5′ GACAGCCGGGGGACACCAUAUACAGACUACGAUA 3′ (SEQ.ID.NO. 187).

The first and second terminal stretches of nucleotides of Type C hepcidin binding nucleic acids comprise four (238-C4-004, 238-C4-011, 238-C4-012, 238-C4-013, 238-C4-014), five (238-C4-003), 238-C4-005, 238-C4-005, 238-C4-006, 238-C4-007, 238-C4-008, 238-C4-009, 238-C4-010, 238-C4-024, 238-C4-025, 238-C4-062), six (238-C4-002) or seven (238-C4-001, 238-E3-001, 238-F2-001, 238-A4-001, 238-E1-001) nucleotides, whereby the stretches optionally hybridize with each other, whereby upon hybridization a double-stranded structure is formed. This double-stranded structure can consists of four to seven basepairs. However, such hybridization is not necessarily given in the molecule.

Combining the first and second terminal stretches of nucleotides of all tested Type C hepcidin binding nucleic acids the generic formula for the first terminal stretch of nucleotides and for the second terminal stretch of nucleotides are 5′X₁X₂X₃SBSN 3′ (first terminal stretch of nucleotides) and 5′NSVSX₄X₅X₆ 3′ (second terminal stretch of nucleotides), wherein X₁ is A or absent, X₂ is G or absent, X₃ is R or absent, X₄ is Y or absent, X₅ is C or absent, X₆ is U or absent,

preferably
a) X₁ is A, X₂ is G, X₃ is R, X₄ is Y, X₅ is C, and X₆ is U or
b) X₁ is absent, X₂ is G, X₃ is R, X₄ is Y, X₅ is C, and X₆ is U or
c) X₁ is A, X₂ is G, X₃ is R, X₄ is Y, X₅ is C, and X₆ is absent or
d) X₁ is absent, X₂ is G, X₃ is R, X₄ is Y, X₅ is C, and X₆ is absent or
e) X₁ is absent, X₂ is absent, X₃ is R, X₄ is Y, X₅ is C, and X₆ is absent or
f) X₁ is absent, X₂ is G, X₃ is R, X₄ is Y, X₅ is absent, and X₆ is absent or
g) X₁ is absent, X₂ is absent, X₃ is R or absent, X₄ is Y or absent, X₅ is absent, and X₆ is absent.

However, the best binding Type C hepcidin binding nucleic acids comprise the following combinations of first and 3′-terminal stretches of nucleotides:
a) 238-C4-001, 238-E3-001: 5′ AGGCUCG 3′ (first terminal stretch of nucleotides) and 5′ CGGGCCU 3′ (second terminal stretch of nucleotides),
b) 238-F2-001: 5′ AGGCCCG 3′ (first terminal stretch of nucleotides) and 5′ CGGGCCU 3′ (second terminal stretch of nucleotides),
c) 238-A4-001: 5′ AGGCUUG 3′ (first terminal stretch of nucleotides) and 5′ CGAGCCU 3′ (second terminal stretch of nucleotides),
d) 238-E1-001: 5′ AGACUUG 3′ (first terminal stretch of nucleotides) and 5′ CGAGUCU 3′ (second terminal stretch of nucleotides),
e) 238-C4-002: 5′ GGCUCG 3′ (first terminal stretch of nucleotides) and 5′ CGGGCC 3′ (second terminal stretch of nucleotides),
f) 238-C4-002: 5′ GGCCG 3′ (first terminal stretch of nucleotides) and 5′ CGGCC 3′ (second terminal stretch of nucleotides)
g) 238-C4-010: 5′ GCGCG 3′ (first terminal stretch of nucleotides) and 5′ CGCGC 3′ (second terminal stretch of nucleotides).

In order to prove the functionality of hepcidin binding nucleic acids as spiegelmers, hepcidin binding nucleic acids 238-C4-006 was synthesized as spiegelmers comprising an Amino-group at its 5′-end. To the amino-modified Spiegelmers 238-C4-001-5′-Amino a 40 kDa PEG-moiety was coupled leading to Type C hepcidin binding nucleic acids 238-C4-006-5′-PEG. Synthesis and PEGyation of the spiegelmer is described in Example 2.

The equilibrium binding constant K_{D} of spiegelmer 238-C4-006-5′-PEG was determined by surface plasmon resonance measurement (FIG. 12): 0.76 nM.

### 1.4 Other Hepcidin Binding Nucleic Acids

As depicted in FIG. 9 other hepcidin binding nucleic acids that are not related to Type A, B and C hepcidin binding nucleic acids are shown. The binding affinities of these hepcidin nucleic acids were determined by Plasmon resonance measurement as well as by competitive binding experiments vs. Type A hepcidin binding nucleic acid 229-G1-001. All nucleic acids showed weaker binding affinity than Type A hepcidin binding nucleic acid 229-G1-001 (FIG. 9).

### EXAMPLE 2

### Synthesis and Derivatization of Aptamers and Spiegelmers

### Small Scale Synthesis

Aptamers (D-RNA nucleic acids) and spiegelmers (L-RNA nucleic acids) were produced by solid-phase synthesis with an ABI 394 synthesizer (Applied Biosystems, Foster City, Calif., USA) using 2′TBDMS RNA phosphoramidite chemistry (Damha and Ogilvie, 1993). rA(N-Bz)-, rC(Ac)-, rG(N-ibu)-, and rU-phosphoramidites in the D- and L-configuration were purchased from ChemGenes, Wilmington, Mass. Aptamers and spiegelmers were purified by gel electrophoresis.

### Large Scale Synthesis Plus Modification

Spiegelmers were produced by solid-phase synthesis with an ÄktaPilot100 synthesizer (Amersham Biosciences; General Electric Healthcare, Freiburg) using 2′TBDMS RNA phosphoramidite chemistry (Damha and Ogilvie, 1993). L-rA(N-Bz), LrC(Ac)-, LrG(N-ibu)-, and L-rU-phosphoramidites were purchased from ChemGenes, Wilmington, Mass. The different 5′-amino-modifier, e.g. the 5′-amino-HEG-HEG linker, was purchased from American International Chemicals Inc. (Framingham, Mass., USA). Synthesis of the unmodified or 5′-Amino-modified spiegelmers were started on L-riboG, LriboC, LriboA or L-riboU modified CPG pore size 1000 Å (Link Technology, Glasgow, UK. For coupling (15 min per cycle), 0.3 M benzylthiotetrazole (CMS-Chemicals, Abingdon, UK) in acetonitrile, and 3.5 equivalents of the respective 0.1 M phosphoramidite solution in acetonitrile was used. An oxidation-capping cycle was used. Further standard solvents and reagents for oligonucleotide synthesis were purchased from Biosolve (Valkenswaard, NL). The spiegelmers were synthesized DMT-ON; after deprotection, it was purified via preparative RP-HPLC (Wincott et al., 1995) using Source15RPC medium (Amersham). The 5′-DMT-group was removed with 80% acetic acid (30 min at RT). Subsequently, aqueous 2 M NaOAc solution was added and the spiegelmers was desalted by tangential-flow filtration using a 5 K regenerated cellulose membrane (Millipore, Bedford, Mass.).

### PEGylation of Spiegelmers

In order to prolong the spiegelmer's plasma residence time in vivo, spiegelmers was covalently coupled to a 40 kDa polyethylene glycol (PEG) moiety at 5′-end.

### 5′-PEGylation of Spiegelmers

For PEGylation (for technical details of the method for PEGylation see European patent application EP 1 306 382), the purified 5′-amino modified spiegelmers were dissolved in a mixture of H₂O (2.5 ml), DMF (5 ml), and buffer A (5 ml; prepared by mixing citric acid. H₂O [7 g], boric acid [3.54 g], phosphoric acid [2.26 ml], and 1 M NaOH [343 ml] and adding water to a final volume of 1 l; pH=8.4 was adjusted with 1 M HCl).

The pH of the spiegelmer solution was brought to 8.4 with 1 M NaOH. Then, 40 kDa PEG-NHS ester (Jenkem Technology, Allen, Tex., USA) was added at 37° C. every 30 min in six portions of 0.25 equivalents until a maximal yield of 75 to 85% was reached. The pH of the reaction mixture was kept at 8-8.5 with 1 M NaOH during addition of the PEG-NHS ester.

The reaction mixture was blended with 4 ml urea solution (8 M), and 4 ml buffer B (0.1 M triethylammonium acetate in H₂O) and heated to 95° C. for 15 min. The PEGylated Spiegelmer was then purified by RP-HPLC with Source 15RPC medium (Amersham), suing an acetonitrile gradient (buffer B; buffer C: 0.1 M triethylammonium acetate in acetonitrile). Excess PEG eluted at 5% buffer C, PEGylated spiegelmer at 10-15% buffer C. Product fractions with a purity of >95% (as assessed by HPLC) were combined and mixed with 40 ml 3 M NaOAC. The PEGylated Spiegelmer was desalted by tangential-flow filtration (5 K regenerated cellulose membrane, Millipore, Bedford, Mass.).

### EXAMPLE 3

### Determination of Binding Constants to Hepcidin (Pull-Down Assay)

### Direct Pull-Down Assay

The affinity of hepcidin binding nucleic acids was measured as aptamers (D-RNA nucleic acids) to biotinylated human D-Hepcidin-25 (SEQ.ID.No. 7) in a pull down assay format at 37° C. Aptamers were 5′-phosphate labeled by T4 polynucleotide kinase (Invitrogen, Karlsruhe, Germany) using [γ-³²P]-labeled ATP (Hartmann Analytic, Braunschweig, Germany). The specific radioactivity of labeled aptamers was 200,000-800,000 cpm/pmol. Aptamers were incubated after de- and renaturation at 20 pM concentration at 37° C. in selection buffer (20 mM Tris-HCl pH 7.4; 137 mM NaCl; 5 mM KCl; 1mM MgCl₂; 1 mM CaCl₂; 0.1% [w/vol] Tween-20) together with varying amounts of biotinylated human D-hepcidin for 2-12 hours in order to reach equilibrium at low concentrations. Selection buffer was supplemented with 10 μg/ml human serum albumin (Sigma-Aldrich, Steinheim, Germany), and 10 μg/ml yeast RNA (Ambion, Austin, USA) in order to prevent adsorption of binding partners to surfaces of used plasticware or the immobilization matrix. The concentration range of biotinylated human D-hepcidin was set from 32 pM to 500 nM; total reaction volume was 1 ml. Biotinylated human D-hepcidin and complexes of aptamer and biotinylated human D-hepcidin were immobilized on 6 μl NeutrAvidin or Streptavidin Ultralink Plus particles (Thermo Scientific, Rockford, USA) which had been preequilibrated with selection buffer and resuspended in a total volume of 12 μl. Particles were kept in suspension for 30 min at the respective temperature in a thermomixer. Immobilized radioactivity was quantitated in a scintillation counter after detaching the supernatant and appropriate washing. The percentage of binding was plotted against the concentration of biotinylated human D-hepcidin and dissociation constants were obtained by using software algorithms (GRAFIT; Erithacus Software; Surrey U.K.) assuming a 1:1 stoichiometry.

### Aptamer Competitive Pull-Down Assay

In order to compare different aptamers of hepcidin binding nucleic acids, a competitive ranking assay was performed. For this purpose the most affine aptamer available was radioactively labeled (see above) and served as reference. After de- and renaturation it was incubated at 37° C. with biotinylated human D-hepcidin in 0.8 ml selection buffer at conditions that resulted in around 5-10% binding to the biotinylated human D-hepcidin-25 after immobilization on NeutrAvidin agarose or Streptavidin Ultralink Plus (both from Thermo Scientific) and washing without competition. An excess of de- and renatured non-labeled D-RNA aptamer variants was added to different concentrations (e.g. 10, 50 and 250 nM) with the labeled reference aptamer to parallel binding reactions. The aptamers to be tested competed with the reference aptamer for target binding, thus decreasing the binding signal in dependence of their binding characteristics. The aptamer that was found most active in this assay could then serve as a new reference for comparative analysis of further aptamer variants.

### Spiegelmer Competitive Pull-Down Assay

In addition, the competitive pull-down assay was performed to analyse the affinity of hepcidin binding spiegelmers. For this purpose spiegelmers binding to biotinylated human L-hepcidin-25 were applied. The addition of two additional guanosine residues in the D-configuration at the 5′-end of the spiegelmers enabled the radioactive labeling of the spiegelmers by T4 polynucleotide kinase (see above). After de- and renaturation the labeled spiegelmer and a set of 5-fold dilutions ranging from 0.032 to 500 nM of competitor molecules (such different species of hepcidin, truncated versions of hepcidin or spiegelmers; see below) were incubated with a constant amount of biotinylated human L-hepcidin in 0.8 ml selection buffer at 37° C. for 2-4 hours. The chosen peptide concentration should cause final binding of approximately 5-10% radiolabeled Spiegelmer at the lowest competitor concentration. In one version of the competitive pull-down assay an excess of de- and renatured non-labeled L-RNA spiegelmer variants served as competitors, whereas unmodified as well as PEGylated forms were tested. In another assay approach non-biotinylated human L-hepcidin-25, marmoset L-hepcidin-25 or rat L-hepcidin-25) or non-biotinylated N-terminal truncated L-hepcidin-20 and L-hepcidin-22 competed against the biotinylated L-hepcidin for spiegelmer binding. After immobilization of biotinylated L-hepcidin-25 and the bound Spiegelmers on 1.5-3 μl Streptavidin Ultralink Plus matrix (Thermo Scientific, Rockford, USA), washing and scintillation counting (see above), the normalized percentage of bound radiolabeled Spiegelmer was plotted against the corresponding concentration of competitor molecules. The resulting dissociation constant was calculated employing the GraFit Software.

### EXAMPLE 4

### Binding Analysis by Surface Plasmon Resonance Measurement

The Biacore 2000 instrument (Biacore AB, Uppsala, Sweden) was used to analyze binding of the aptamers of the hepcidin binding nucleic acids against biotinylated human D-hepcidin-25 and of the spiegelmers of the hepcidin binding nucleic acids against biotinylated L-hepcidin-20, as well as human, rat and mouse D-hepcidin 25.

the instrument was set to a enduring temperature of 37° C. Before the start of each experiment the Biacore was cleaned using the DESORB method according to the manufacturer's instructions. After docking a maintenance chip, the instrument was consecutively primed with DESORB solution 1 (0.5% sodium dodecyl sulphate, SDS), DESORB solution 2 (50 mM glycine, pH 9.5) and finally degassed MilliQ water. Subsequently the SANATIZE method was run with 0.1M NaOCl and the system was primed afterwards with MilliQ water.

The biotinylated human D-hepcidin 25, human L-hepcidin-20, as well as human, rat, and mouse L-hepcidin 25 (all peptides from BACHEM, custom synthesis) were dissolved in water with 1 mg/ml fatty-acid free BSA at a concentration of 1 mM in a screw lock vial and stored at 4° C. until use.

After docking a sensor chip with a carboxymethylated dextran matrix (Sensor Chip CM5, GE, BR-1000-14), the Biacore instrument was primed with MilliQ water followed by HBS-EP buffer (0.01 M HEPES buffer [pH 7.4], 0.15 M NaCl, with 0.005% Surfactant P20; GE, BR-1001-88) and equilibrated until a stable baseline was observed. The flow cells (FCs) were immobilized beginning from flow cell 4 to flow cell 1 to avoid carry-over of peptides to other flow cells.

100 μl of a 1:1 mixture of 0.4M EDC (1-ethyl-3-(3-dimethylaminopropyl)carbodiimide in H₂O; GE, BR-1000-50) and 0.1M NHS (N-hydroxysuccinimide in H₂O; GE, BR1000-50) were injected using the QUICKINJECT command at a flow of 10 μl/min. Activation of the flow cell was monitored by an increase in RU after NHS/EDC injection (typically 500-600 RU for CM5 chips). Soluble Neutravidin was dissolved in water to a concentration of 1 mg/ml, diluted in HBS-EP to 50 μg/ml and subsequently injected using the MANUALINJECT command at a flow of 10 μl/min. The maximal observed amount of covalently immobilized Neutravidin was about 10,000-15,000 RU. The flow cells were blocked with a injection 70 μl of 1 M ethanolamine hydrochloride (GE, BR-1000-50) at a flow of 10 μl/min; typically non-covalently bound peptide/protein is removed by this procedure. Non-covalently coupled Neutravidin was removed by an injection of 10-30 μl of a 50 mM NaOH solution. Biotinylated human D-hepcidin 25, human L-hepcidin 20, as well as human, rat and mouse biotinylated L-hepcidin 25 was directly diluted to a final concentration of 10-20 nM in HBS-EP buffer and vortexed immediately. 1000 μl of this sample was transferred to 9 mm glass vial (Glass Vials, 9 mm, GE, BR-1002-07) and injected using the MANUALINJECT command at a flow of 10 μl/min. For binding experiments up to 5000 response units (RU) of biotinylated human D-hepcidin 25, human L-hepcidin 20, as well as human, rat and mouse L-hepcidin 25 and for kinetic evaluations 500-1500 RU were immobilized on the flow cell. Subsequently the flow cell was washed with 1M NaCl (Ambion, Cat.No.AM9759) to avoid carry over of biotinylated human D-hepcidin 25, human L-hepcidin 20, as well as human, rat and mouse L-hepcidin 25 due to unspecific interaction of biotinylated human D-hepcidin 25, human L-hepcidin 20, as well as human, rat and mouse L-hepcidin 25 with the Biacore tubing and other surfaces. FC1 served as blocked control flow cell.

Finally all sensor flow cells (beginning from FC1 to FC4) were blocked by injection 20 μl of a saturated biotin solution (Biotin, Sigma-Aldrich B-4501 Lot 68H1373) diluted 1:10 in HBS-EP buffer at a flow of 20 μl/min. The sensor chip was primed twice with degased running buffer (20 mM Tris pH 7.4; 150 mM NaCl; 5 mM KCl, 1 mM MgCl₂, 1 mM CaCl₂ and 0.1% Tween20) and equilibrated at 30 μl/min until the baseline appeared stable.

Typically for analytical purpose, the aptamers/spiegelmers of hepcidin binding nucleic acid were diluted in water to a stock concentration of 100 μM (quantification by UV measurement), heated up to 95° C. for 30 seconds in a water bath or thermo mixer and snap cooled on ice to assure a homogenous dissolved solution.

Kinetic parameters and dissociation constants were evaluated by a series of aptamer injections at concentrations of 1000, 500, 250, 125, 62.5, 31.25, 25.63, 7.8, 3.9 and 0 nM diluted in running buffer. In all experiments, the analysis was performed at 37° C. using the Kinject command defining an association time of 360 and a dissociation time of 360 seconds at a flow of 30 μl/min. The assay was double referenced, whereas FC1 served as (blocked) surface control (bulk contribution of each aptamer concentration) and a series of buffer injections without analyte determined the bulk contribution of the buffer itself. Data analysis and calculation of dissociation constants (KD) was done with the BIAevaluation 3.0 software (BIACORE AB, Uppsala, Sweden) using the Langmuir 1:1 stochiometric fitting algrorithm.

### EXAMPLE 5

### Activity of a Hepcidin Binding Spiegelmer in Vivo

The current concept of anemia of chronic diseases it that hepcidin synthesis and release is stimulated by pro-inflammatory cytokines, especially IL-6, in hepatocytes. Hepcidin than binds to the different cell types expressing the iron transporter ferroportin. This interaction induces an internalisation and a degradation of the hepcidin-ferroportin complex followed by a serum iron decrease. A chronic reduction of serum iron negatively impairs erythropoiesis and finally manifests in anemia. The known property of human hepcidin-25 to induce a serum iron decrease in mice (Rivera, 2005) was utilized as a model for anaemia of inflammation. To test the activity of Spiegelmers in vivo a state of hypoferremia was induced in C57BL/6 mice with a human-hepcidin-25. To characterise the spiegelmers in this model, animals received a prophylactic treatment with the Spiegelmer to block the effect of hu-hepcidin.

### Method

Female C57Bl/6 mice (Elevage Janvier, France, six weeks old, n=6-7 per group) received a single intravenous injection of a anti-hepcidin spiegelmer (10-20 ml/kg body weight) or vehicle (5% glucose, 10-20 ml/kg body weight). After thirty minutes synthetic human hepcidin-25 (Bachem, Weil am Rhein, Germany, Cat No. H-5926) at a dose of 1-2 mg/kg body weight was injected intraperitoneally (10 ml/kg body weight). Blood was collected two hours after the hepcidin injection. Serum and plasma samples were obtained for iron determination and complete blood count, respectively. For each animal the serum iron, haemoglobin, hematocrit, white blood cell count, erythrocyte count, thrombocyte count, mean corpuscular volume, and mean corpuscular haemoglobin values were determined.

### Results

Injection of synthetic human-hepcidin-25 leads to a rapid reduction of serum iron. Two hours after injection the serum iron concentration was reduced to 56% of the value of the vehicle treated mice. These in vivo findings are in line with the data published by Rivera et al. (Ribera et al.), who reported a reduction to ca. 25% in a very similar experiment with a higher hepcidin dose. The decrease in serum iron is completely blocked (98% of control) by application of spiegelmer 239-D4-008-5′-PEG prior to injection of human hepcidin as depicted in FIG. 14.

### EXAMPLE 6

### Activity of a Hepcidin Binding Spiegelmer in Cynomomolgus Monkeys Stimulated with Human Interleukin-6

The dominant role of Interleukin-6 (IL-6) in anemia of chronic diseases was demonstrated with the IL-6 receptor antibody tocilizumab. Treatment with this antibody showed efficacy in patients with Castleman disease (Nishimoto, 2008) and also in an arthritis model in cynomologus monkeys (Hashizume, 2009). The known property of IL-6 to induce hepcidin secretion subsequently resulting in anemia in non-human primates was utilized as another model for anaemia of inflammation (Asano, 1990; Klug 1994). Instead of the parameter haemoglobin the serum iron content was selected as endpoint to show efficacy of anti-hepcidin spiegelmers. A state of hypoferremia was induced in cynomolgus monkeys with human-recombinant IL-6. This model was important to show that anti-hepcidin spiegelmers also bind the endogenous hepcidin, as in all other experiments a synthetic human hepcidin was used. To test the activity of spiegelmers in vivo a state of hypoferremia was induced in cynomolgus monkeys with human-recombinant IL-6. To characterise the spiegelmers in this model, animals received a prophylactic treatment with the Spiegelmer to block the effect of cynomolgus-hepcidin.

### Method

Male cynomolgus monkeys (Robert C., Hartelust, Tilburg, The Netherlands) 34 to 38 months old, n=3 per group) received a single intravenous injection of a anti-hepcidin spiegelmer (1 ml/kg body weight) or vehicle (5% glucose, 1 ml/kg body weight). After thirty minutes recombinant human IL-6 (Miltenyi Biotech, Bergisch Gladbach, Germany) at a dose of 10 μg/kg body weight was injected subcutaneously (1 ml/kg body weight). Blood was collected eight hours after the IL-6 injection. Serum and plasma samples were obtained for iron determination and complete blood count, respectively. For each animal the serum iron, haemoglobin, hematocrit, white blood cell count, erythrocyte count, thrombocyte count, mean corpuscular volume, and mean corpuscular haemoglobin values were determined.

### Results

Injection of recombinant human IL-6 leads to a reduction of serum iron. Eight hours after injection the serum iron concentration was reduced to 27% of the predose value of the vehicle/IL-6 treated monkeys. The decrease in serum iron is completely blocked by application of spiegelmer 238-D4-008-5′-PEG prior to injection of human IL-6 as depicted in FIG. 15.

### EXAMPLE 17

### Use of a Hepcidin Binding Nucleic in Hemodiafiltration

In the experiments a hepcidin binding nucleic acid with a molecular mass of 14.602 Dalton (Spiegelmer NOX-H94-002, a derivative of Spiegelmer NOX-H94 without PEG modification), a highly permeable dialysis membrane and highly convective conditions were employed to show the elimination of the Spiegelmer NOX-H94-002 in an in vitro hemodiafiltration setting with human whole blood.

### Methods

Spiegelmer NOX-H94-002 was dissolved in 5% glucose resulting in a 10 mg/ml stock solution.

The dialyzer used was Phylther HF17SD, Fa. Bellco (Mirandola, Italy, 1.7 m² surface area, steam-sterilized, Lot: 0903170005). The experiments were performed with human donor blood according to the set-up as described in EN 1283. In brief, a pool of donor whole blood (10 U/ml heparin) was standardized at the start of the experiments to reach a hematocrit of 32±2% (actual mean: 29.8±0.2). Mean actual total protein concentration was 67±1 g/l at the start of the experiments. Dialyzers were rinsed with 1 L saline in single pass and 1 L of saline in recirculation (100 ml/min, 20 min) by the Nikkiso dialysis monitor DBB-03 (Nikkiso Medical GmBH, Hamburg, Germany).

Three hemodiafiltration experiments were performed with 524 ml blood standardized as described above. Q_{B} (Q_{B} is the blood flow rate) was 500 ml/min. Q_{D} (Q_{D} is the dialysate flow rate) was 700 ml/min and Q_{F} (Q_{F} is the filtrate flow rate) was 100 ml/min. After start of the dialysis experiment, conditions were allowed to equilibrate for 28 minutes. After 28 minutes, the spiegelmer stock solution previously mixed with concentrated hemofiltrate of uremic patients containing the reference proteins β₂-microglobin (Mᵣ=11,800), cystatin C (Mᵣ=13,300), myoglobin (Mᵣ=17,600) and retinol binding protein (Mᵣ=21,000) was added to the circuit. Thus, assuming a molar mass of 14.602, spiegelmer concentration in the blood pool at 28 minutes was 3 μM. At time points 30, 32 and 34 minutes a sample was taken from dialyser inlet (Cₐ) and outlet (Cᵥ). Additional samples were taken from the dialyzer inlet (Cₐ) at 31, 33 and 35 minutes possibly leading to an improved accuracy of the measurement by a kinetic clearance calculation. An aliquot was taken from the total dialysate volume (15 L), which was collected between 30 and 60 minutes of the experiment, to allow determination of recovery for the spiegelmer.

Protein concentrations were measured by laser nephelometry (BN ProSpec, Dade-Behring, Marburg, Germany). In the table and figure below clearance of proteins β₂-microglobin, cystatin C, myoglobin and retinol binding protein are expressed as mean clearance calculated from n=9 (three times points in three experiments each).

The Spiegelmer concentrations were measured by a sandwich hybridisation method as described in WO/2008/052774 (Example 9).

Plasma water clearance (a/v clearance) was calculated according to the equation

Clₚₗₐₛₘₐ=Q_{B}(1−0.0107×TP)((SPC×Hct+(1−Hct))((Cₐ−Cᵥ)/Cₐ)+(Q_{UF}×Cᵥ/Cₐ)) [ml/min]

in which Q_{B} is the blood flow rate, Q_{UF} is the ultrafiltration rate (10 ml/min), Cᵥ and Cₐ are the venous (dialyzer outlet) and arterial (dialyzer inlet) solute concentration, Hct is the patient's hematocrit at the time of sampling and TP is the total protein concentration [g/L] at the same time point. To account for solute shifts from blood cells, solute partition coefficients (abbr. SPC) were assumed as 0 (for β₂ m, cystatin c, myoglobin, retinal binding protein).

### Results

Mean clearance values of the marker proteins and the spiegelmer from three experiments

| | Plasma clearance | | Plasma clearance | |
|---|---|---|---|---|
| | a/v | | kinetic | |
| | (mean of n = 3) | | (mean of n = 3) | |
| β₂-Microglobin | 91 ± 14 | ml/min | 150 ± 6 | ml/min |
| (M₁ 11.800) | | | | |
| Cystatin C | 91 ± 14 | ml/min | 129 ± 6 | ml/min |
| (M₁ 13.300) | | | | |
| Myoglobin | 66 ± 7 | ml/min | 121 ± 30 | ml/min |
| (M₁ 17.600) | | | | |
| Retinal binding | 36 ± 9 | ml/min | 26 ± 1 | ml/min |
| protein | | | | |
| (M₁ 21.000) | | | | |
| NOX-H94002 | 33 ± 42 | ml/min | 26 ± 19 | ml/min |
| (M₁ 14.602) | | | | |

50 ± 10% of the spiegelmer NOX-H94-002 was found in dialysate collected between 30 and 60 min.

### Discussion

Clearances for the proteins β₂-microglobin (Mᵣ 11.800), cystatin C (Mᵣ 13.300), myoglobin (Mᵣ 17.600) and retinol binding protein (Mᵣ 21.000) were determined to show the validity of the dialysis experiments. Protein clearances are in the range known for the dialyzer used. The spiegelmer does not behave as a globular protein explaining clearance values in the range of retinol binding protein rather than in the range of the protein marker with a similar molecular weight (e.g. cystatin c).

Under the conditions used clearance and removal into dialysate was clearly measurable for the spiegelmer NOX-H94-002 with a molecular mass of 14.602. The amount recovered from dialysate was about 50%.

### EXAMPLE 8

### Depletion of Hepcidin in Hepcidin Containing Human Serum Samples Using Spiegelmer NOX-H94 Immobilised on a Solid Support

To test whether a hepcidin binding nucleic acid can be used in an apheresis procedure, hepcidin binding spiegelmer NOX-H94-3xHEG-amino was used. NOX-H94-3xHEG-amino is a 5′ amino modified derivative of NOX-H94 Spiegelmer sequence, with three hexaethylene glycol moieties inserted between the Spiegelmer part and the amino functional group of the molecule. This amino functionality is used as a handle to covalently attach the Spiegelmer to a sepharose support.

Within the experiments as described herein, hepcidin binding spiegelmer NOX-H94-3xHEG-amino was coupled to an activated solid support (in this case a NHS-ester activated sepharose support). After the conjugation process, the support was treated with an agent to block the activated sites on the support so that no covalent binding of the biological solution contents can occur. The support was then washed and the amounts of the NOX-H94-3xHEG-amino in the conjugation, blocking and washing solutions were determined by calculating the optical density units (abbr. ODs) of NOX-H94-3xHEG-amino (FIG. 16). As N-hydroxysuccinimide, a by-product of the conjugation reaction also has a 260 nm absorbance, it was necessary to analyse the the conjugation, blocking and washing solutions via anion-exchange HPLC chromatogram (FIG. 17) to determine the percentage of 260 nm absorbance attributable to the remaining in solution (FIG. 16). The amount of hepcidin binding Spiegelmer NOX-H94-3xHEG-amino conjugated to the support was calculated of the difference of the amount of the starting material NOX-H94-3xHEG-amino and the amounts of NOX-H94-3xHEG-amino determined in the conjugation, blocking and washing solutions (FIG. 16).

The support was then incubated with human serum spiked with different quantities of hepcidin. After incubation the amounts of hepcidin were determined in the supernatant using a competitive ELISA assay. The supernatant was removed from the beads by compacting the beads via centrifugation, and carefully removing the supernatant. The support was subsequently washed three times with delipidated, defibrinated, hepcidin depleted human serum. These washing solutions were combined and the hepcidin amounts determined.

Determination of unspecific hepcidin binding to the support was examined by repeating the hepcidin-spike human serum incubations with sepharose support with no NOX-H94-3xHEG-amino conjugated thereto. The activated NHS ester groups on these solid support samples were blocked by treatment with ethanolamine so that no covalent binding of biological solution contents could occur. The block support was incubated with human serum spiked with the same quantities of hepcidin as used for the support with NOX-H94-3xHEG-amino conjugated thereto. After incubation hepcidin amounts in the supernatant were determined using a competitive ELISA assay. The supernatant was removed from the beads by compacting the beads via centrifugation, and carefully removing the supernatant. The support was subsequently washed three times with delipidated, defibrinated, hepcidin depleted human serum. These washing solutions were combined and the hepcidin amounts determined.

### Protocol

### Immobilisation of Spiegelmer on Sepharose Support

200 μl of NHS-activated Sepharose 4 Fast Flow (16-23 μmol NHS/ml medium, #17-0906-01, GE Healthcare, Bio-Sciences AB, Sweden) was washed with cold 1 mM HCl (1 mL). The HCl solution was removed by compacting the support via centrifugation and removing the supernatant. To this support was immediately added 100 μl of a 0.314 μmol/ml solution (31.4 nmol, 12.3 ODs) of spiegelmer NOX-H94-3xHEG-amino in Theorell & Stenhagen's Universal buffer pH 8.25 (33 mM Sodium Citrate, 33 nM Sodium Phosphate, 57 mM Sodium Borate, pH 8.25). The suspension was incubated on a Eppendorf Thermomixer Comfort machine (Eppendorf, Hamburg, Germany) at 25° C. for two hours whereupon the support was compacted and the supernatant removed. The supernatant, the conjugation solution, was retained for further analysis. The support was then washed with PBS buffer (3×100 μl) followed by a pre-wash with a solution containing 0.5M ethanolamine, 0.5M NaCl pH 8.3 (1×300 μl). Supernatants, the combined washings solution, were retained for further analysis. The NHS-ester activated sites on the support were blocked by adding 300 μl of a solution containing 0.5M ethanolamine, 0.5M NaCl pH 8.3 and incubating on a Eppendorf Thermomixer Comfort machine (Eppendorf, Hamburg, Germany) at 25° C. for 2 hours whereupon the support was compacted and the supernatant, the blocking solution, removed and retained for analysis. The support was then washed with sterile water (1×100 μl), followed by 3×100 μl alternating washes with 0.1 M Tris-HCl pH 8 (Applichem, BioChemica) and 0.1M sodium acetate pH 5. The support was finally washed with 1×300 μl sterile water, the combined washing solutions. Supernatants of the conjugation solution, the combined washings and the blocking solutions that were retained for further analysis were treated as follows: Optical density units at 260 nm were measured (FIG. 16), then samples were analysed with anion exchange HPLC (DNA-Pac PA200 column, 4×250 mm, from Dionex Mobile Phase A; 25 mM Tris, 1 mM EDTA, 10 mM NaClO₄, 10% ACN; Mobile phase B; 25 mM Tris, 1 mM EDTA, 500 mM NaClO₄, 10% ACN. Gradient 20-55% B in 19 min, column temperature 85° C.) to determine the percentage of ODs in the sample attributable to NOX-H94-3xHEG-amino, see FIG. 17 for an exemplary chromatogram. The amount of hepcidin binding Spiegelmer NOX-H94-3xHEG-amino conjugated to the support was calculated of the difference of the amount of the starting material NOX-H94-3xHEG-amino and the amounts of NOX-H94-3xHEG-amino determined in the conjugation, blocking and washing solutions (FIG. 16).

### Preparation of Blocked Supports to be Used as Controls

1 mL of NHS-activated Sepharose 4 Fast Flow (16-23 μmol NHS/ml medium, #17-0906-01, GE Healthcare) was placed in a 5 mL disposable column (Qiagen, Germany). The Isopropanol storage solution was removed and the support was washed with 2 mL of a solution containing 0.5M ethanolamine, 0.5M NaCl pH 8.3. The solution was removed and another 2 mL of a solution containing 0.5M ethanolamine, 0.5M NaCl pH 8.3 was added. The column was sealed and shaken for 4 h whereupon the solution was removed and the support washed with sterile water (1×2 mL) followed by 4×3 mL alternating washes with 0.1 M Tris-HCl pH 8 (Applichem, BioChemica) and 0.1M sodium acetate pH 5. Finally the support was washed with 1×3 mL of sterile water.

### Preparation of Hepcidin Spiked Human Serum

Human pool plasma (lithium-Heparin-plasma, pool from 15 female and 15 male plasma individuals, PLHI-123-E, Lot # E708238 and E808238, Sera Laboratories Industries, UK) was spiked with Hepcidin 25 (Lot # 102854, Bachem) to a final concentration of 50 nM and 500 nM, respectively, using a 10 μM stock solution of Hepcidin in water.

Incubation of Supports with Hepcidin-Spiked Human Serum

15 μL of support coupled with NOX-H94-3xHEG-amino and 15 μL of Ethanolamine blocked sepharose support (no NOX-H94-3xHEG-amino coupled) were incubated with each 150 μL of plasma spiked with 50 nM or 500 nM Hepcidin (referred as “Load Hepcidin”, see FIG. 20B) under agitation in a thermoshaker at 25° C. at 550 rpm for 2 h. After incubation the samples were centrifuged in a capsule centrifuge for 5 sec and the supernatants were transferred into a fresh tube (referred as “Unbound hepcidin”, see FIG. 20 B). the supports were washed three times with 50 μL double charcoal stripped hepcidin free defibrinated, delipidized human serum (#1005.HSdes, Nova Biologics, USA) and the wash fractions were collected and combined in a fresh tube (referred as “Wash”, see FIG. 20B).

### Determination of Hepcidin Levels in Human Serum

Hepcidin was determined in the samples using the ELISA Kit from Bachem (Hepcidin-25, human, EIA, Extraction-free kit for human serum/plasma Bachem Ltd., Peninsula Laboratories, LLC, S-1337). The ELISA was performed according to the protocol II of manufacturer; only the deviations are listed below. A ten point standard curve was prepared with serial dilution in Assay Diluent (BD Bio BD sciences, OptEIA™ #555213) with 10% blank matrix as described in FIG. 18. The quality control samples were prepared as 10× stock solutions with hepcidin spiked in blank matrix as shown in FIG. 19. Quality control samples are designed according to calibration curve as high level (HiQC), medium level (MeQC) and low level (LoQC) sample supplemented by the upper limit of quantification (ULOQ) sample and lower limit of quantification (LLOQ) sample. For the assay the stocks were diluted with Assay Diluent without matrix 1 in 10 (25 μL stock solution+225 μL Assay diluent). The test samples were diluted according to their expected hepcidin concentration as depicted in FIG. 19.

### Results

Hepcidin binding nucleic Spiegelmer NOX-H94-3xHEG-amino was efficiently conjugated to the support in a covalent manner using the protocol described in the experimental section. Active sites on the support were blocked using an ethanolamine solution following the manufacturer's protocol. This was followed by a washing of the support. The amount of NOX-H94-3xHEG-amino that was attached to the support could be determined by measuring the optical density units (abbr. ODs) at 260 nm (FIG. 16), whereby, a N-hydroxysuccinimide, a by-product of the conjugation reaction also has a 260 nm absorbance, the conjugation solution, the combined washing solution and the blocking solution were additionally analysed via anion-exchange HPLC chromatogram (FIG. 17) to determine the percentage of 260 nm absorbance attributable to the remaining NOX-H94-3xHEG-amino in solution (FIGS. 16 an 17). As can be seen, only a small percentage of the measured ODs correspond to the NOX-H94-3xHEG-amino starting material (FIG. 16) but to N-hydroxysuccinimide: 9.4 OD (corresponds to 24 nmol) of the 12.3 OD (corresponds to 31.4 nmol) of the starting material NOX-H94-3xHEG-amino was conjugated to NHS-activated sepharose. That corresponds to a loading of 120 nmol NOX-H94-3xHEG on one mL sepharose (FIG. 16).

15 μL of support coupled with NOX-H94-3xHEG-amino (corresponds to 3 nmol conjugated NOX-H94-3xHEG-amino) were incubated with each 150 μL of plasma spiked with 50 nM (corresponds to 0.0075 nmol hepcidin) or 500 nM hepcidin (corresponds to 0.075 nmol hepcidin) (see FIG. 20A, see line “reaction A” and “reaction B”). Ethanolamine blocked sepharose support (NOX-H94 3xHEG amino modified coupled) was prepared according to the protocol and used as a control to monitor the unspecific binding of hepcidin to the support. 15 μL of ethanolamine blocked sepharose support (no NOX-H94-3xHEG-amino coupled) were incubated with each 150 μL of plasma spiked with 50 nM (corresponds to 0.0075 nmol hepcidin) or 500 nM hepcidin (corresponds to 0.075 nmol hepcidin) (see FIG. 20A, see line “reaction C” and “reaction D”). The amounts of hepcidin determined after incubation the plasma spiked with hepcidin with 15 μL of support coupled with NOX-H94-3xHEG-amino or within 15 μL of ethanolamine blocked sepharose support (no NOX-H94-3xHEG-amino coupled) are summarised in FIG. 20B. As can be readily see in FIG. 20B, the determined amounts of hepcidin in the supernatants (“unbound %” hepcidin and “wash % hepcidin”) show that the support coupled with NOX-H94-3xHEG-amino almost completely depletes the hepcidin from plasma spiked with hepcidin (see line “reaction A” and “reaction B”, FIG. 20B)). In contrast for the ethanolamine blocked sepharose support (no NOX-H94-3xHEG-amino conjugated thereto) only low hepcidin binding capacity was shown hepcidin (see line “reaction C” and “reaction D”, FIG. 20B) Therefore, these results clearly show that immobilised NOX-H94-3xHEG-amino effectively depletes hepcidin from biological solutions.

In summary, by applying the principles above, it was demonstrated that NOX-H94-3xHEG-amino immobilised on support can efficiently deplete Hepcidin in biological solutions.

### REFERENCES

Abboud S, Haile D J (2000) A novel mammalian iron-regulated protein involved in intracellular iron metabolism. J Biol Chem 275(26): 19906-19912
Altschul S F, Gish W, Miller W, Myers E W, Lipmann D J (1990), Basic local alignment search tool J Mol Biol 215 (3): 403-10.
Altschul S F, Madden T L, Schaffer A A, Zhang J, Zhang Z, Miller W, Lipman D J (1997), Gapped BLAST and PSI-BLAST: a new generation of protein database search programs. Nucleic Acid Research 25(17):3389-402.
Andrews N C (2008) Forging a field: the golden age of iron biology. Blood 112(2): 219-230
Asano S, Okano A, Ozawa K, Nakahata T, Ishibashi T, Koike K, Kimura H, Tanioka Y, Shibuya A, Hirano T (1990) In vivo effects of recombinant human interleukin-6 in primates: stimulated production of platelets. Blood 75(8): 1602-1605
Constante M, Jiang W, Wang D, Raymond V A, Bilodeau M, Santos M M (2006) Distinct requirements of Hfe in basal and induced hepcidin levels in iron overload and inflammation. Am J Physiol Gastrointest Liver Physiol 291(2): G229-237
De Domenico I, Ward D M, Langelier C, Vaughn M B, Nemeth E, Sundquist W I, Ganz T, Musci G, Kaplan J (2007) The molecular mechanism of hepcidin-mediated ferroportin down-regulation. Mol Biol Cell 18(7): 2569-2578
Donovan A, Brownlie A, Zhou Y, Shepard J, Pratt S J, Moynihan J, Paw B H, Drejer A, Barut B, Zapata A, Law T C, Brugnara C, Lux S E, Pinkus G S, Pinkus J L, Kingsley P D, Palis J, Fleming M D, Andrews N C, Zon L I (2000) Positional cloning of zebrafish ferroportin1 identifies a conserved vertebrate iron exporter. Nature 403 (6771): 776-781
Frazer D M, Wilkins S J, Millard K N, McKie A T, Vulpe C D, Anderson G J (2004) Increased hepcidin expression and hypoferraemia associated with an acute phase response are not affected by inactivation of HFE. Br J Haematol 126(3): 434-436
Hashizume M, Uchiyama Y, Horai N, Tomosugi N, Mihara M (2009) Rheumatol Int July 29, DOI 10.1007/s00296-009-1075-4.
Hermanson, G. T., (1996) Bioconjugate Techniques, Academic Press, 1st edition (Jan. 22, 1996)
Klug S, Neubert R, Stahlmann R, Thiel R, Ryffel B, Car B D, Neubert D (1994) Effects of recombinant human interleukin 6 (rhIL-6) in marmosets (Callithrix jacchus). I. General toxicity and hematological changes. Arch Toxicol 68(10): 619-31
Klussmann, S. (eds.), The Aptamer Handbook, 1. Edition—February 2006, Wiley-VCH, Weinheim
Klusser W. (2000) Chemically modified nucleic acid aptamers for in vitro selections: evolving evolution. J Biotechnol 74(1): 27-38.
Krause A, Neitz S, Magert H J, Schulz A Forssmann W G, Schulz-Knappe P, Adermann K (2000) LEAP-1, a novel highly disulfide-bonded human peptide, exhibits antimicrobial activity. FEBS Lett 480(2-3): 147-150
McKie A T, Marciani P, Rolfs A, Brennan K, Wehr K, Barrow D, Miret S, Bomford A, Peters T J, Farzaneh F, Hediger M A, Hentze M W, Simpson R J (2000) A novel duodenal iron-regulated transporter, IREG1, implicated in the basolateral transfer of iron to the circulation. Mol Cell 5(2): 299-309
Needleman & Wunsch (1970), A general method applicable to the search for similarities in the amino acid sequence of two proteins, J Mol Biol 48(3): 443-53.
Nemeth E, Rivera S, Gabayan V, Keller C, Taudorf S, Pedersen B K, Ganz T (2004a) IL-6 mediates hypoferremia of inflammation by inducing the synthesis of the iron regulatory hormone hepcidin. J Clin Invest 113(9): 1271-1276
Nemeth E, Tuttle M S, Powelson J, Vaughn M B, Donovan A, Ward D M, Ganz T, Kaplan J (2004b) Hepcidin regulates cellular iron efflux by binding to ferroportin and inducing its internalization. Science 306(5704): 2090-2093
McGinnis S., Madden T. L., et al. (2004) BLAST: at the core of a powerful and diverse set of sequence analysis tools. Nucleic Acids Res. 32 (Web Server issue): W20-5.
Nicolas G, Bennoun M, Devaux I, Beaumont C, Grandchamp B, Kahn A, Vaulont S (2001) Lack of hepcidin gene expression and severe tissue iron overload in upstream stimulatory factor 2 (USF2) knockout mice. Proc Natl Acad Sci U S A 98(15): 8780-8785
Nicolas G, Bennoun M, Porteu A, Mativet S, Beaumont C, Grandchamp B, Sirito M, Sawadogo M, Kahn A, Vaulont S (2002a) Severe iron deficiency anemia in transgenic mice expressing liver hepcidin. Proc Natl Acad Sci U S A 99(7): 4596-4601
Nicolas G, Chauvet C, Viatte L, Danan J L, Bigard X, Devaux I, Beaumont C, Kahn A, Vaulont S (2002b) The gene encoding the iron regulatory peptide hepcidin is regulated by anemia, hypoxia, and inflammation. J Clin Invest 110(7): 1037-1044
Nicolas G, Viatte L, Lou D Q, Bennoun M, Beaumont C, Kahn A, Andrews N C, Vaulont S (2003) Constitutive hepcidin expression prevents iron overload in a mouse model of hemochromatosis. Nat Genet 34(1): 97-101
Nishimoto N, Terao K, Mima T, Nakahara H, Takagi N, Kakehi T (2008) Mechanisms and pathologic significances in increase in serum interleukin-6 (IL-6) and soluble IL-6 receptor after administration of an anti-IL-6 receptor antibody, tocilizumab, in patients with rheumatoid arthritis and Castleman disease. Blood 112(10): 3959-3964.
Park C H, Valore E V, Waring A J, Ganz T (2001) Hepcidin, a urinary antimicrobial peptide synthesized in the liver. J Biol Chem 276(11): 7806-7810
Pearson & Lipman (1988), Improved tools for biological sequence comparison. Proc Nat. Acad Sci USA 85:2444.
Rivera S, Nemeth E, Gabayan V, Lopex M A, Farshidi D, Ganz T (2005) synthetic hepcidin causes rapid dose-dependent hypoferremia and is concentrated in ferroportin-containing organs. Blood 106(6): 2196-2199
Roetto A, Papanikolaou G, Politou M, Alberti F, Girelli D, Christakis J, Loukopoulos D, Camaschella C (2003) Mutant antimicrobial peptide hepcidin is associated with severe juvenile hemochromatosis. Nat Genet 33(1): 21-22
Smith & Waterman (1981), Adv. Appl. Math. 2:482
Venkatesan N., Kim S. J., et al. (2003) Novel phosphoramidite building blocks in synthesis and applications toward modified oligonucleotides. Curr Med Chem 10(19): 1973-91.
Weiss G (2008) Iron metabolism in the anemia of chronic disease. Biochim Biophys Acta
Weiss G, Goodnough L T (2005) Anemia of chronic disease. N Engl J Med 352(10): 1011-1023

The features of the present invention disclosed in the specification, the claims, the sequence listing and/or the drawings may both separately and in any combination thereof be material for realizing the invention in various forms thereof.

1-104. (canceled)

105. A method for reducing the level of hepcidin in a body fluid from a subject, comprising
a) providing a nucleic acid that binds hepcidin,
b) bringing the nucleic acid into contact with the body fluid under conditions that allow the nucleic acid to bind the hepcidin forming a complex of hepcidin and the nucleic acid, and
c) removing the complex from the body fluid or removing the hepcidin from the body fluid.

106. The method according to claim 105, wherein the nucleic acid comprises in 5→3′ direction a first terminal stretch of nucleotides, a central stretch of nucleotides and a second terminal stretch of nucleotides, or a second terminal stretch of nucleotides, a central stretch of nucleotides and a first terminal stretch of nucleotides, wherein the central stretch of nucleotides comprises 32 to 40 nucleotides or 32 to 35 nucleotides.

107. The method according to claim 106, wherein the central stretch of nucleotides comprises 5′ RKAUGGGAKUAAGUAAAUGAGGRGUWGGAGGAAR 3′ (SEQ ID NO:182) or 5′ RKAUGGGAKAAGUAAAUGAGGRGUWGGAGGAAR 3′ (SEQ ID NO:183).

108. The method according to claim 106, wherein the central stretch of nucleotides comprises five to eight nucleotides, and the second terminal stretch of nucleotides comprises five to eight nucleotides.

109. The method according to claim 106, wherein the central stretch of nucleotides comprises 5′ X₁X₂X₃SBSBC3′ and the second terminal stretch of nucleotides comprises 5′ GVBVYX₄X₅X₆ 3′; wherein X₁ is A or absent, X₂ is G or absent, X₃ is B or absent, X₄ is S or absent, X₅ is C or absent, and X₆ is U or absent.

110. The method according to claim 105, wherein the nucleic acid comprises SEQ ID NO: 115, SEQ ID NO: 116, SEQ ID NO: 117, SEQ ID NO: 118, SEQ ID NO: 119, SEQ ID NO: 121, SEQ ID NO: 142, SEQ ID NO: 144, SEQ ID NO: 146, SEQ ID NO: 148, SEQ ID NO: 151, SEQ ID NO: 152, SEQ ID NO: 175 or SEQ ID NO: 176.

111. The method according to claim 106, wherein the central stretch of nucleotides comprises 5′ GRCRGCCGGVGGACACCAUAUACAGACUACKAUA 3′ (SEQ ID NO:185) or 5′ GRCRGCCGGVAGGACACCAUAUACAGACUACKAUA 3′ (SEQ ID NO:186).

112. The method according to claim 111, wherein the first terminal stretch of nucleotides comprises four to seven nucleotides, and the second terminal stretch of nucleotides comprises four to seven nucleotides.

113. The method according to claim 106, wherein the first terminal stretch of nucleotides comprises 5′ X₁X₂X₃SBSN3′ and the second terminal stretch of nucleotides comprises 5′ NSVS₄X₅X₆ 3′, wherein X₁ is A or absent, X₂ is G or absent, X₃ is R or absent, X₄ is Y or absent, X₅ is C or absent, and X₆ is U or absent.

114. The method according to claim 105, wherein the nucleic acid molecule comprises SEQ ID NO: 122, SEQ ID NO: 123, SEQ ID NO: 124, SEQ ID NO: 125, SEQ ID NO: 126, SEQ ID NO: 154, SEQ ID NO: 159, SEQ ID NO: 163 or SEQ ID NO: 174.

115. The method according to claim 106, wherein the central stretch of nucleotides comprises in 5′→3′ direction: Box A, linking stretch of nucleotides and Box B; or Box B, linking stretch of nucleotides and Box A, wherein Box A comprises 5′ WAAAGUWGAR 3′ (SEQ ID NO:188), the linking stretch of nucleotides comprises ten to eighteen nucleotides, and Box B comprises 5′ RGMGUGWKAGUKC 3′ (SEQ ID NO:189) .

116. The method according to claim 115, wherein the linking stretch of nucleotides comprises in 5′→3′ direction, a first linking substretch of nucleotides, a second linking substretch of nucleotides and a third linking substretch of nucleotides, wherein the first linking substretch of nucleotides comprises three to six nucleotides, the second linking substretch of nucleotides comprises three to five nucleotides and the third linking substretch of nucleotides comprises three to six nucleotides.

117. The method according to claim 116, wherein
a) the first linking substretch of nucleotides comprises 5′ GGAC 3′, 5′ GGAU 3′ or 5′ GGA 3′, and the third linking substretch of nucleotides comprises 5′ GUCC 3′;
b) the first linking substretch of nucleotides comprises 5′ GCAG 3′ and the third linking substretch of nucleotides comprises 5′ CUGC 3′;
c) the first linking substretch of nucleotides comprises 5′ GGGC 3′ and the third linking substretch of nucleotides comprises 5′ GCCC 3′;
d) the first linking substretch of nucleotides comprises 5′ GAC 3′ and the third linking substretch of nucleotides comprises 5′ GUC 3′;
e) the first linking substretch of nucleotides comprises 5′ ACUUGU 3′ and the third linking substretch of nucleotides comprises 5′ GCAAGU 3′ or 5′ GCAAGC 3′; or
f) the first linking substretch of nucleotides comprises 5′ UCCAG 3′ and the third linking substretch of nucleotides comprises 5′ CUGGA 3′.

118. The method according to claim 116, wherein the second linking substretch of nucleotides comprises 5′ VBAAW 3′, 5′ AAUW 3′ or 5′ NBW 3′.

119. The method according to claim 115, wherein the first terminal stretch of nucleotides comprises four to seven nucleotides, and the second terminal stretch of nucleotides comprises four to seven nucleotides.

120. The method according to claim 115, wherein the first terminal stretch of nucleotides comprises 5′ X₁X₂X₃BKBK3′ and the second terminal stretch of nucleotides comprises 5′ MVVV₄X₅X₆ 3′, wherein X₁ is G or absent, X₂ is S or absent, X₃ is V or absent, X₄ is B or absent, X₅ is S or absent, and X₆ is C or absent.

121. The method according to claim 105, wherein the nucleic acid molecule comprises SEQ ID NO: 29, SEQ ID NO: 33, SEQ ID NO: 34, SEQ ID NO: 39, SEQ ID NO: 40, SEQ ID NO: 41, SEQ ID NO: 43, SEQ ID NO: 46, SEQ ID NO: 137, SEQ ID NO: 138, SEQ ID NO: 139, SEQ ID NO: 140, SEQ ID NO: 141 or SEQ ID NO: 173.

122. The method according to claim 105, wherein the nucleic acid molecule comprises SEQ ID NO: 127, SEQ ID NO: 128, SEQ ID NO: 129, SEQ ID NO: 130, or SEQ ID NO: 131.

123. The method according to claim 105, wherein the nucleic acid molecule comprises a modification group, wherein excretion rate of the nucleic acid molecule comprising the modification group from an organism is decreased as compared to excretion rate of the nucleic acid molecule that binds hepcidin not comprising the modification group.

124. The method according to claim 105, wherein the nucleic acid molecule comprises a modification group, wherein the nucleic acid molecule comprising the modification group has an increased retention time in an organism as compared to the nucleic acid molecule that binds hepcidin not comprising the modification group.

125. The method according to claim 123 or 124, wherein the modification group is biodegradable.

126. The method according to claim 123 or 124, the modification group is selected from the group consisting of a linear poly(ethylene)glycol, a branched poly(ethylene)glycol, a hydroxyethyl starch, a peptide, a protein, a polysaccharide, a sterol, a polyoxypropylene, a polyoxyamidate, a poly(2-hydroxyethyl)-L-glutamine and a polyethylene glycol.

127. The method according to claim 105, wherein the nucleic acid molecule comprises an L-nucleic acid.

128. The method according to claim 105, wherein said reducing comprises a treatment and/or a prevention of a disease.

129. The method according to claim 105, wherein the nucleic acid molecule is immobilized on a support.

130. The method according to claim 105, wherein the body fluid is brought into contact with a semi-permeable membrane that separates the body fluid from a dialysate, wherein hepcidin and/or a complex of hepcidin and the nucleic acid molecule diffuses through the semi-permeable membrane from the body fluid to the dialysate, thereby reducing the level of hepcidin in the body fluid.

131. A method for preparing a nucleic acid molecule that specifically binds hepcidin immobilised on a support comprising reacting the nucleic acid molecule that binds hepcidin and an activated support to form a bond between a 3′ end, a 5′ end or both of the nucleic acid molecule and the support.

132. The method according to claim 105, wherein said bringing and said removing comprise a medical device.

133. The method according to claim 105, wherein said subject comprises a mammal.

134. The method according to claim 105, wherein said subject comprises an anemia.


SEQUENCE LISTING (216 sequences)

SEQ ID NO: 1 - protein, 25 aa, Homo sapiens
  PEPTIDE (1)..(25)
DTHFPICIFCCGCCHRSKCGMCCKT

SEQ ID NO: 2 - protein, 25 aa, Macaca mulatta
  PEPTIDE (1)..(25)
DTHFPICIFCCGCCHRSKCGMCCKT

SEQ ID NO: 3 - protein, 25 aa, Macaca fascicularis
  PEPTIDE (1)..(25)
DTHFPICIFCCGCCHRSKCGMCCKT

SEQ ID NO: 4 - protein, 25 aa, Sus scrofa
  PEPTIDE (1)..(25)
DTHFPICIFCCGCCRKAICGMCCKT

SEQ ID NO: 5 - protein, 25 aa, Mus musculus
  PEPTIDE (1)..(25)
DTNFPICIFCCKCCNNSQCGICCKT

SEQ ID NO: 6 - protein, 25 aa, Rattus norvegicus
  PEPTIDE (1)..(25)
DTNFPICLFCCKCCKNSSCGLCCIT

SEQ ID NO: 7 - protein, 25 aa, Homo sapiens
  misc_feature: D-peptide
DTHFPICIFCCGCCHRSKCGMCCKT

SEQ ID NO: 8 - protein, 20 aa, Homo sapiens
  PEPTIDE (1)..(20)
ICIFCCGCCHRSKCGMCCKT

SEQ ID NO: 9 - protein, 22 aa, Homo sapiens
  PEPTIDE (1)..(22)
FPICIFCCGCCHRSKCGMCCKT

SEQ ID NO: 10 - RNA, 48 nt, Artificial
  synthetic
gcacucguaaaguagagggacccaguccggcgugauagugccgagugc

SEQ ID NO: 11 - RNA, 48 nt, Artificial
  synthetic
gcacuuguaaaguagagggacccaguccggcgugauagugccgagugc

SEQ ID NO: 12 - RNA, 48 nt, Artificial
  synthetic
gcauucguaaaguagagggacccaguccggcgugauagugccgagugc

SEQ ID NO: 13 - RNA, 48 nt, Artificial
  synthetic
gcacucguaaaguagagggaccuaguccggcgugauagugccgggugc

SEQ ID NO: 14 - RNA, 48 nt, Artificial
  synthetic
gcacucguaaaguagagggaccuaguccggcgugauagugccgagugc

SEQ ID NO: 15 - RNA, 48 nt, Artificial
  synthetic
gcacucguaaaguagagggacucaguccggcgugauagugccgagugc

SEQ ID NO: 16 - RNA, 48 nt, Artificial
  synthetic
gcacucguaaaguagagggauacaguccggcgugauagugacgagugc

SEQ ID NO: 17 - RNA, 48 nt, Artificial
  synthetic
cguguguaaaguagaggcagguaaucugcggaguguuaguuccacacg

SEQ ID NO: 18 - RNA, 48 nt, Artificial
  synthetic
cgcguguaaaguagaggcagguaaucugcggaguguuaguuccacacg

SEQ ID NO: 19 - RNA, 48 nt, Artificial
  synthetic
cguguguaaaguagaggcaggcaaucugcggaguguuaguuccacacg

SEQ ID NO: 20 - RNA, 45 nt, Artificial
  synthetic
cguguguaaaguagaggacaauugucggcgugauagugccacacg

SEQ ID NO: 21 - RNA, 45 nt, Artificial
  sybthetic
gcuguguaaaguagaggacaauugucggcgugauagugccacagc

SEQ ID NO: 22 - RNA, 45 nt, Artificial
  Synthetic
cguguguaaaguagaggacaauagucggcgugagagugccacacg

SEQ ID NO: 23 - RNA, 48 nt, Artificial
  Synthetic
cgugaaaaguagaaacuugucgaaagcaaguagcgugauagugccacg

SEQ ID NO: 24 - RNA, 48 nt, Artificial
  Synthetic
cgugcuggcgugauagugcuccagguucuggauaaaguagagagcacg

SEQ ID NO: 25 - RNA, 48 nt, Artificial
  Synthetic
cgugcgaaggagugauaaguguuucugacuuucuuccagacucccacg

SEQ ID NO: 26 - RNA, 46 nt, Artificial
  synthetic
cacucguaaaguagagggacccaguccggcgugauagugccgagug

SEQ ID NO: 27 - RNA, 46 nt, Artificial
  synthetic
cgcgcguaaaguagagggacccaguccggcgugauagugccgcgcg

SEQ ID NO: 28 - RNA, 44 nt, Artificial
  synthetic
gcgcguaaaguagagggacccaguccggcgugauagugccgcgc

SEQ ID NO: 29 - RNA, 48 nt, Artificial; identical to SEQ ID NO: 10
  synthetic

SEQ ID NO: 30 - RNA, 48 nt, Artificial; identical to SEQ ID NO: 11
  synthetic

SEQ ID NO: 31 - RNA, 48 nt, Artificial; identical to SEQ ID NO: 12
  synthetic

SEQ ID NO: 32 - RNA, 48 nt, Artificial; identical to SEQ ID NO: 13
  synthetic

SEQ ID NO: 33 - RNA, 48 nt, Artificial; identical to SEQ ID NO: 14
  synthetic

SEQ ID NO: 34 - RNA, 48 nt, Artificial; identical to SEQ ID NO: 15
  synthetic

SEQ ID NO: 35 - RNA, 48 nt, Artificial; identical to SEQ ID NO: 16
  synthetic

SEQ ID NO: 36 - RNA, 48 nt, Artificial; identical to SEQ ID NO: 17
  synthetic

SEQ ID NO: 37 - RNA, 48 nt, Artificial; identical to SEQ ID NO: 18
  synthetic

SEQ ID NO: 38 - RNA, 48 nt, Artificial; identical to SEQ ID NO: 19
  synthetic

SEQ ID NO: 39 - RNA, 45 nt, Artificial; identical to SEQ ID NO: 20
  synthetic

SEQ ID NO: 40 - RNA, 45 nt, Artificial; identical to SEQ ID NO: 21
  synthetic

SEQ ID NO: 41 - RNA, 45 nt, Artificial; identical to SEQ ID NO: 22
  synthetic

SEQ ID NO: 42 - RNA, 48 nt, Artificial; identical to SEQ ID NO: 23
  synthetic

SEQ ID NO: 43 - RNA, 48 nt, Artificial; identical to SEQ ID NO: 24
  synthetic

SEQ ID NO: 44 - RNA, 48 nt, Artificial; identical to SEQ ID NO: 25
  synthetic

SEQ ID NO: 45 - RNA, 46 nt, Artificial; identical to SEQ ID NO: 26
  synthetic

SEQ ID NO: 46 - RNA, 46 nt, Artificial; identical to SEQ ID NO: 27
  synthetic

SEQ ID NO: 47 - RNA, 44 nt, Artificial; identical to SEQ ID NO: 28
  synthetic

SEQ ID NO: 48 - RNA, 48 nt, Artificial; identical to SEQ ID NO: 10
  synthetic

SEQ ID NO: 49 - RNA, 44 nt, Artificial
  synthetic
aggcguaaaguagaggggcugagcccggcguguuagugccgccu

SEQ ID NO: 50 - RNA, 44 nt, Artificial
  synthetic
aggcguaaaguagagggacguaguccggcgugauagugccgccu

SEQ ID NO: 51 - RNA, 48 nt, Artificial
  synthetic
cguguguaaaguagaggcagauaaucugcggaguguuaguuccacacg

SEQ ID NO: 52 - RNA, 48 nt, Artificial
  synthetic
cgugaaaaguagaaacuugucgaaagcaagcagcgugauagugccacg

SEQ ID NO: 53 - RNA, 48 nt, Artificial
  synthetic
cgugaaaaguugaaauuuguuggaaucaagcagggauauagugccacg

SEQ ID NO: 54 - RNA, 49 nt, Artificial
  synthetic
agcgugucguaugggauaaguaaaugaggaguuggaggaagggugcgcu

SEQ ID NO: 55 - RNA, 50 nt, Artificial
  synthetic
agcgugucguaugggauuaaguaaaugaggaguuggaggaagggcaugcu

SEQ ID NO: 56 - RNA, 49 nt, Artificial
  synthetic
agugugucguaugggauaaguaaaugagggguuggaggaaggaugcgcu

SEQ ID NO: 57 - RNA, 49 nt, Artificial
  synthetic
agugugucauaugggauaaguaaaugaggaguuggaggaaaggcaugcu

SEQ ID NO: 58 - RNA, 49 nt, Artificial
  synthetic
agcgugccggaugggauaaguaaaugaggaguuggaggaagggugcgcu

SEQ ID NO: 59 - RNA, 49 nt, Artificial
  synthetic
agcgugccguaugggauaaguaaaugaggaguaggaggaaggguacgcu

SEQ ID NO: 60 - RNA, 49 nt, Artificial
  synthetic
agcgcgccguaugggagaaguaaaugaggaguuggaggaagggcgcgcu

SEQ ID NO: 61 - RNA, 48 nt, Artificial
  synthetic
aggcucggacagccgggggacaccauauacagacuacgauacgggccu

SEQ ID NO: 62 - RNA, 48 nt, Artificial
  synthetic
aggcucggacggccgggggacaccauauacagacuacuauacgggccu

SEQ ID NO: 63 - RNA, 48 nt, Artificial
  synthetic
aggcccggacagccgggggacaccauauacagacuacuauacgggccu

SEQ ID NO: 64 - RNA, 48 nt, Artificial
  synthetic
aggcuugggcggccgggggacaccauauacagacuacuauacgagccu

SEQ ID NO: 65 - RNA, 48 nt, Artificial
  synthetic
agacuugggcagccgggggacaccauauacagacuacgauacgagucu

SEQ ID NO: 66 - RNA, 49 nt, Artificial
  synthetic
cgggcgccauagaccguuauuaagcacuguaacuaccgaaccgcgcccg

SEQ ID NO: 67 - RNA, 45 nt, Artificial
  synthetic
cgggcgccauagaccguuaacuacauaacuaccgaaccgugcccg

SEQ ID NO: 68 - RNA, 45 nt, Artificial
  synthetic
cgggcgcuaccgaacccacuaaaaccagugcauagaccgcgcccg

SEQ ID NO: 69 - RNA, 41 nt, Artificial
  synthetic
cgggcgcuaccgaaccgucacgaagaccauagaccgcgccg

SEQ ID NO: 70 - RNA, 41 nt, Artificial
  synthetic
cgagcgcaaccgaaccucuacccagacauagaccgcgcccg

SEQ ID NO: 71 - RNA, 47 nt, Artificial
  synthetic
gcacucguaaaguagagggaccaguccggcgugauagugccgagugc

SEQ ID NO: 72 - RNA, 43 nt, Artificial
  synthetic
guguguaaaguagaggacaauugucggcgugauagugccacac

SEQ ID NO: 73 - RNA, 43 nt, Artificial
  synthetic
gcguguaaaguagaggacaauugucggcgugauagugccacgc

SEQ ID NO: 74 - RNA, 43 nt, Artificial
  synthetic
gcgcguaaaguagaggacaauugucggcgugauagugccgcgc

SEQ ID NO: 75 - RNA, 44 nt, Artificial
  synthetic
cguguguaaaguagaggacaauugucggcgugauagugccacac

SEQ ID NO: 76 - RNA, 45 nt, Artificial
  synthetic
gccguguaaaguagaggacaauugucggcgugauagugccacggc

SEQ ID NO: 77 - RNA, 45 nt, Artificial
  synthetic
gcgguguaaaguagaggacaauugucggcgugauagugccaccgc

SEQ ID NO: 78 - RNA, 45 nt, Artificial
  synthetic
gcugcguaaaguagaggacaauugucggcgugauagugccgcagc

SEQ ID NO: 79 - RNA, 45 nt, Artificial
  synthetic
gcuggguaaaguagaggacaauugucggcgugauagugccccagc

SEQ ID NO: 80 - RNA, 45 nt, Artificial
  synthetic
gcggcguaaaguagaggacaauugucggcgugauagugccgccgc

SEQ ID NO: 81 - RNA, 46 nt, Artificial
  synthetic
gcgcgcguaugggauuaaguaaaugaggaguuggaggaaggcgcgc

SEQ ID NO: 82 - RNA, 45 nt, Artificial
  synthetic
gcgcgcguaugggauaaguaaaugaggaguuggaggaaggcgcgc

SEQ ID NO: 83 - RNA, 44 nt, Artificial
  synthetic
ggcgcguaugggauuaaguaaaugaggaguuggaggaaggcgcc

SEQ ID NO: 84 - RNA, 43 nt, Artificial
  synthetic
ggcgcguaugggauaaguaaaugaggaguuggaggaaggcgcc

SEQ ID NO: 85 - RNA, 46 nt, Artificial
  synthetic
ggugucguaugggauuaaguaaaugaggaguuggaggaagggcauc

SEQ ID NO: 86 - RNA, 45 nt, Artificial
  synthetic
ggugucguaugggauaaguaaaugaggaguuggaggaagggcauc

SEQ ID NO: 87 - RNA, 44 nt, Artificial
  synthetic
gcgccguaugggauuaaguaaaugaggaguuggaggaagggcgc

SEQ ID NO: 88 - RNA, 43 nt, Artificial
  synthetic
gcgccguaugggauaaguaaaugaggaguuggaggaagggcgc

SEQ ID NO: 89 - RNA, 46 nt, Artificial
  synthetic
ggcgccguaugggauuaaguaaaugaggaguuggaggaagggcgcc

SEQ ID NO: 90 - RNA, 45 nt, Artificial
  synthetic
ggcgccguaugggauaaguaaaugaggaguuggaggaagggcgcc

SEQ ID NO: 91 - RNA, 46 nt, Artificial
  synthetic
ggcgucguaugggauuaaguaaaugaggaguuggaggaagggcgcc

SEQ ID NO: 92 - RNA, 45 nt, Artificial
  synthetic
ggcgucguaugggauaaguaaaugaggaguuggaggaagggcgcc

SEQ ID NO: 93 - RNA, 46 nt, Artificial
  synthetic
ggcucggacagccgggggacaccauauacagacuacgauacgggcc

SEQ ID NO: 94 - RNA, 44 nt, Artificial
  synthetic
gcucggacagccgggggacaccauauacagacuacgauacgggc

SEQ ID NO: 95 - RNA, 42 nt, Artificial
  synthetic
cucggacagccgggggacaccauauacagacuacgauacggg

SEQ ID NO: 96 - RNA, 44 nt, Artificial
  synthetic
gcccggacagccgggggacaccauauacagacuacgauacgggc

SEQ ID NO: 97 - RNA, 44 nt, Artificial
  synthetic
ggccggacagccgggggacaccauauacagacuacgauacggcc

SEQ ID NO: 98 - RNA, 44 nt, Artificial
  synthetic
gcggagacagccgggggacaccauauacagacuacgauauccgu

SEQ ID NO: 99 - RNA, 44 nt, Artificial
  synthetic
aggcugacagccgggggacaccauauacagacuacgauaggccu

SEQ ID NO: 100 - RNA, 44 nt, Artificial
  synthetic
ggccugacagccgggggacaccauauacagacuacgauaaggcu

SEQ ID NO: 101 - RNA, 44 nt, Artificial
  sysnthetic
gcgcggacagccgggggacaccauauacagacuacgauacgcgc

SEQ ID NO: 102 - RNA, 42 nt, Artificial
  synthetic
gccggacagccgggggacaccauauacagacuacgauacggc

SEQ ID NO: 103 - RNA, 42 nt, Artificial
  synthetic
ggcggacagccgggggacaccauauacagacuacgauacgcc

SEQ ID NO: 104 - RNA, 42 nt, Artificial
  synthetic
ggccgacagccgggggacaccauauacagacuacgauaggcc

SEQ ID NO: 105 - RNA, 42 nt, Artificial
  synthetic
gcgcgacagccgggggacaccauauacagacuacgauagcgc

SEQ ID NO: 106 - RNA, 44 nt, Artificial
  synthetic
ggccggacagccggaggacaccauauacagacuacgauacggcc

SEQ ID NO: 107 - RNA, 44 nt, Artificial
  synthetic
ggccggacagccggcggacaccauauacagacuacgauacggcc

SEQ ID NO: 108 - RNA, 45 nt, Artificial
  synthetic
ggccggacagccgggaggacaccauauacagacuacgauacggcc

SEQ ID NO: 109 - RNA, 13 nt, Artificial
  synthetic
uccagguucugga

SEQ ID NO: 110 - RNA, 44 nt, Artificial; identical to SEQ ID NO: 49
  synthetic

SEQ ID NO: 111 - RNA, 44 nt, Artificial; identical to SEQ ID NO: 50
  synthetic

SEQ ID NO: 112 - RNA, 48 nt, Artificial; identical to SEQ ID NO: 51
  synthetic

SEQ ID NO: 113 - RNA, 48 nt, Artificial; identical to SEQ ID NO: 52
  synthetic

SEQ ID NO: 114 - RNA, 48 nt, Artificial; identical to SEQ ID NO: 53
  synthetic

SEQ ID NO: 115 - RNA, 49 nt, Artificial; identical to SEQ ID NO: 54
  synthetic

SEQ ID NO: 116 - RNA, 50 nt, Artificial; identical to SEQ ID NO: 55
  synthetic

SEQ ID NO: 117 - RNA, 49 nt, Artificial; identical to SEQ ID NO: 56
  synthetic

SEQ ID NO: 118 - RNA, 49 nt, Artificial; identical to SEQ ID NO: 57
  synthetic

SEQ ID NO: 119 - RNA, 49 nt, Artificial; identical to SEQ ID NO: 58
  synthetic

SEQ ID NO: 120 - RNA, 49 nt, Artificial; identical to SEQ ID NO: 59
  synthetic

SEQ ID NO: 121 - RNA, 49 nt, Artificial; identical to SEQ ID NO: 60
  synthetic

SEQ ID NO: 122 - RNA, 48 nt, Artificial; identical to SEQ ID NO: 61
  synthetic

SEQ ID NO: 123 - RNA, 48 nt, Artificial; identical to SEQ ID NO: 62
  synthetic

SEQ ID NO: 124 - RNA, 48 nt, Artificial; identical to SEQ ID NO: 63
  synthetic

SEQ ID NO: 125 - RNA, 48 nt, Artificial; identical to SEQ ID NO: 64
  synthetic

SEQ ID NO: 126 - RNA, 48 nt, Artificial; identical to SEQ ID NO: 65
  synthetic

SEQ ID NO: 127 - RNA, 49 nt, Artificial; identical to SEQ ID NO: 66
  synthetic

SEQ ID NO: 128 - RNA, 45 nt, Artificial; identical to SEQ ID NO: 67
  synthetic

SEQ ID NO: 129 - RNA, 45 nt, Artificial; identical to SEQ ID NO: 68
  synthetic

SEQ ID NO: 130 - RNA, 41 nt, Artificial; identical to SEQ ID NO: 69
  synthetic

SEQ ID NO: 131 - RNA, 41 nt, Artificial; identical to SEQ ID NO: 70
  synthetic

SEQ ID NO: 132 - RNA, 47 nt, Artificial; identical to SEQ ID NO: 71
  synthetic

SEQ ID NO: 133 - RNA, 43 nt, Artificial; identical to SEQ ID NO: 72
  synthetic

SEQ ID NO: 134 - RNA, 43 nt, Artificial; identical to SEQ ID NO: 73
  synthetic

SEQ ID NO: 135 - RNA, 43 nt, Artificial; identical to SEQ ID NO: 74
  synthetic

SEQ ID NO: 136 - RNA, 44 nt, Artificial; identical to SEQ ID NO: 75
  synthetic

SEQ ID NO: 137 - RNA, 45 nt, Artificial; identical to SEQ ID NO: 76
  synthetic

SEQ ID NO: 138 - RNA, 45 nt, Artificial; identical to SEQ ID NO: 77
  synthetic

SEQ ID NO: 139 - RNA, 45 nt, Artificial; identical to SEQ ID NO: 78
  synthetic

SEQ ID NO: 140 - RNA, 45 nt, Artificial; identical to SEQ ID NO: 79
  synthetic

SEQ ID NO: 141 - RNA, 45 nt, Artificial; identical to SEQ ID NO: 80
  synthetic

SEQ ID NO: 142 - RNA, 46 nt, Artificial; identical to SEQ ID NO: 81
  synthetic

SEQ ID NO: 143 - RNA, 45 nt, Artificial; identical to SEQ ID NO: 82
  synthetic

SEQ ID NO: 144 - RNA, 44 nt, Artificial; identical to SEQ ID NO: 83
  synthetic

SEQ ID NO: 145 - RNA, 43 nt, Artificial; identical to SEQ ID NO: 84
  synthetic

SEQ ID NO: 146 - RNA, 46 nt, Artificial; identical to SEQ ID NO: 85
  synthetic

SEQ ID NO: 147 - RNA, 45 nt, Artificial; identical to SEQ ID NO: 86
  synthetic

SEQ ID NO: 148 - RNA, 44 nt, Artificial; identical to SEQ ID NO: 87
  synthetic

SEQ ID NO: 149 - RNA, 43 nt, Artificial; identical to SEQ ID NO: 88
  synthetic

SEQ ID NO: 150 - RNA, 46 nt, Artificial; identical to SEQ ID NO: 89
  synthetic

SEQ ID NO: 151 - RNA, 45 nt, Artificial; identical to SEQ ID NO: 90
  synthetic

SEQ ID NO: 152 - RNA, 46 nt, Artificial; identical to SEQ ID NO: 91
  synthetic

SEQ ID NO: 153 - RNA, 45 nt, Artificial; identical to SEQ ID NO: 92
  synthetic

SEQ ID NO: 154 - RNA, 46 nt, Artificial; identical to SEQ ID NO: 93
  synthetic

SEQ ID NO: 155 - RNA, 44 nt, Artificial; identical to SEQ ID NO: 94
  synthetic

SEQ ID NO: 156 - RNA, 42 nt, Artificial; identical to SEQ ID NO: 95
  synthetic

SEQ ID NO: 157 - RNA, 10 nt, Artificial
  synthetic
gacaauaguc

SEQ ID NO: 158 - RNA, 44 nt, Artificial; identical to SEQ ID NO: 96
  synthetic

SEQ ID NO: 159 - RNA, 44 nt, Artificial; identical to SEQ ID NO: 97
  synthetic

SEQ ID NO: 160 - RNA, 44 nt, Artificial; identical to SEQ ID NO: 98
  synthetic

SEQ ID NO: 161 - RNA, 44 nt, Artificial; identical to SEQ ID NO: 99
  synthetic

SEQ ID NO: 162 - RNA, 44 nt, Artificial; identical to SEQ ID NO: 100
  synthetic

SEQ ID NO: 163 - RNA, 44 nt, Artificial; identical to SEQ ID NO: 101
  synthetic

SEQ ID NO: 164 - RNA, 42 nt, Artificial; identical to SEQ ID NO: 102
  synthetic

SEQ ID NO: 165 - RNA, 42 nt, Artificial; identical to SEQ ID NO: 103
  synthetic

SEQ ID NO: 166 - RNA, 42 nt, Artificial; identical to SEQ ID NO: 104
  synthetic

SEQ ID NO: 167 - RNA, 42 nt, Artificial; identical to SEQ ID NO: 105
  synthetic

SEQ ID NO: 168 - RNA, 44 nt, Artificial; identical to SEQ ID NO: 106
  synthetic

SEQ ID NO: 169 - RNA, 44 nt, Artificial; identical to SEQ ID NO: 107
  synthetic

SEQ ID NO: 170 - RNA, 45 nt, Artificial; identical to SEQ ID NO: 108
  synthetic

SEQ ID NO: 171 - RNA, 45 nt, Artificial; identical to SEQ ID NO: 21
  synthetic

SEQ ID NO: 172 - RNA, 48 nt, Artificial; identical to SEQ ID NO: 10
  synthetic

SEQ ID NO: 173 - RNA, 45 nt, Artificial; identical to SEQ ID NO: 21
  synthetic

SEQ ID NO: 174 - RNA, 44 nt, Artificial; identical to SEQ ID NO: 97
  synthetic

SEQ ID NO: 175 - RNA, 46 nt, Artificial; identical to SEQ ID NO: 81
  synthetic

SEQ ID NO: 176 - RNA, 44 nt, Artificial; identical to SEQ ID NO: 87
  synthetic

SEQ ID NO: 177 - RNA, 11 nt, Artificial
  synthetic
ccauacggcgc

SEQ ID NO: 178 - RNA, 13 nt, Artificial
  synthetic
gcgcccuuccucc

SEQ ID NO: 179 - RNA, 46 nt, Artificial; identical to SEQ ID NO: 81
  synthetic

SEQ ID NO: 180 - RNA, 44 nt, Artificial; identical to SEQ ID NO: 87
  synthetic

SEQ ID NO: 181 - RNA, 44 nt, Artificial; identical to SEQ ID NO: 97
  synthetic

SEQ ID NO: 182 - RNA, 34 nt, Artificial
  synthetic
rkaugggakuaaguaaaugaggrguwggaggaar

SEQ ID NO: 183 - RNA, 33 nt, Artificial
  synthetic
rkaugggakaaguaaaugaggrguwggaggaar

SEQ ID NO: 184 - RNA, 34 nt, Artificial
  synthetic
guaugggauuaaguaaaugaggaguuggaggaag

SEQ ID NO: 185 - RNA, 34 nt, Artificial
  synthetic
grcrgccggvggacaccauauacagacuackaua

SEQ ID NO: 186 - RNA, 35 nt, Artificial
  synthetic
grcrgccggvaggacaccauauacagacuackaua

SEQ ID NO: 187 - RNA, 34 nt, Artificial
  synthetic
gacagccgggggacaccauauacagacuacgaua

SEQ ID NO: 188 - RNA, 10 nt, Artificial
  synthetic
waaaguwgar

SEQ ID NO: 189 - RNA, 13 nt, Artificial
  synthetic
rgmgugwkagukc

SEQ ID NO: 190 - RNA, 11 nt, Artificial
  synthetic
gggcugagccc

SEQ ID NO: 191 - RNA, 13 nt, Artificial
  synthetic
gcagauaaucugc

SEQ ID NO: 192 - RNA, 10 nt, Artificial
  synthetic
ggaccagucc

SEQ ID NO: 193 - RNA, 11 nt, Artificial
  synthetic
ggacccagucc

SEQ ID NO: 194 - RNA, 11 nt, Artificial
  synthetic
ggaccuagucc

SEQ ID NO: 195 - RNA, 11 nt, Artificial
  synthetic
ggacucagucc

SEQ ID NO: 196 - RNA, 13 nt, Artificial
  synthetic
gcagguaaucugc

SEQ ID NO: 197 - RNA, 13 nt, Artificial
  synthetic
gcaggcaaucugc

SEQ ID NO: 198 - RNA, 10 nt, Artificial
  synthetic
gacaauuguc

SEQ ID NO: 199 - RNA, 10 nt, Artificial
  synthetic
uaaaguagag

SEQ ID NO: 200 - RNA, 10 nt, Artificial
  synthetic
aaaaguagaa

SEQ ID NO: 201 - RNA, 10 nt, Artificial
  synthetic
aaaaguugaa

SEQ ID NO: 202 - RNA, 12 nt, Artificial
  synthetic
gggauauagugc

SEQ ID NO: 203 - RNA, 13 nt, Artificial
  synthetic
ggcgugauagugc

SEQ ID NO: 204 - RNA, 13 nt, Artificial
  synthetic
ggaguguuaguuc

SEQ ID NO: 205 - RNA, 13 nt, Artificial
  synthetic
ggcgugagagugc

SEQ ID NO: 206 - RNA, 13 nt, Artificial
  synthetic
agcgugauagugc

SEQ ID NO: 207 - RNA, 13 nt, Artificial
  synthetic
ggcguguuagugc

SEQ ID NO: 208 - RNA, 11 nt, Artificial
  synthetic
ggacbyagucc

SEQ ID NO: 209 - RNA, 11 nt, Artificial
  synthetic
ggauacagucc

SEQ ID NO: 210 - RNA, 13 nt, Artificial
  synthetic
gcaggyaaucugc

SEQ ID NO: 211 - RNA, 10 nt, Artificial
  synthetic
gacaauwguc

SEQ ID NO: 212 - RNA, 17 nt, Artificial
  synthetic
acuugucgaaagcaagy

SEQ ID NO: 213 - DNA, 44 bp, artificial
  synthetic
gcgccguaugggauuaaguaaaugaggaguuggaggaagggcgc

SEQ ID NO: 214 - DNA, 11 bp, artificial
  synthetic
ggacguagucc

SEQ ID NO: 215 - DNA, 18 bp, artificial
  synthetic
auuuguuggaaucaagca

SEQ ID NO: 216 - DNA, 34 bp, artificial
  synthetic
grcrgccgggggacaccauauacagacuackaua